(19) United States
(12) Patent Application Publication
(10) Pub. No.: US 2026/0355305 A1
(43) Pub. Date: Nov. 12, 2026

(54) VACCINE USING M2/BM2-DEFICIENT INFLUENZA VECTORS

(71) Applicant: FluGen, Inc., Madison, WI (US)

(72) Inventors: Michael J. MOSER, Madison, WI (US); David J. MARSHALL, Madison, WI (US); Liam I. MARSHALL, Madison, WI (US); Yasuko HATTA, Madison, WI (US); Pamuk BILSEL, Madison, WI (US)

(73) Assignee: FluGen, Inc., Madison, WI (US)

(21) Appl. No.: 19/744,738

(22) Filed: Jul. 16, 2026

Related U.S. Application Data
(62) Division of application No. 18/017,371, filed on Jul. 21, 2021, now Pat. No. 12,728,160.
(60) Provisional application No. 63/054,700, filed on Jul. 21, 2020.

(51) Int. Cl. A61K 39/215; A61K 39/00; C07K 14/11

(52) U.S. Cl. CPC A61K 39/215; C07K 14/11; A61K 2039/543; A61K 2039/70; C12N 2770/20022

(57) ABSTRACT

The invention provides a recombinant virus comprising an influenza viral backbone, wherein the influenza viral backbone comprises PB1, PB2, PA, NP, M, NS, HA, and NA gene segments, wherein at least one of the PB1, PB2, PA, NP, M, NS, HA, and NA gene segments comprises at least one nucleotide sequence that encodes one or more antigens. The invention provides a recombinant virus wherein the antigen is an immunogenic fragment of SARS-COV-2 spike glycoprotein. The invention also provides a pharmaceutical formulation and a method of eliciting an immune response.

### CROSS-REFERENCE TO RELATED APPLICATIONS

This patent application is a divisional of copending U.S. patent application Ser. No. 18/017,371, filed Jan. 20, 2023, which is the U.S. national phase of International Application No. PCT/US2021/042561, filed Jul. 21, 2021, which claims the benefit of U.S. Provisional Application 63/054,700, filed Jul. 21, 2020, each of which are incorporated herein by reference in their entireties for all purposes.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 222,889 Byte Extensible Markup Language (XML) file named “776906-SequenceListing_ST26.xml,” created on Jul. 13, 2026.

### BACKGROUND OF THE INVENTION

Vaccines are important tools for preventing illness from infectious disease. Infectious diseases can infect millions of people worldwide. Thus, it is important to develop vaccines against many different types of diseases and to do so quickly and efficiently. For example, the novel coronavirus disease 2019 (COVID-19) is a global pandemic caused by the newly emerged virus severe acute respiratory syndrome coronavirus 2 (SARS-COV-2). Over 10 million people worldwide have been diagnosed with the disease and hundreds of thousands have died from it. In its severe form, the disease is characterized by acute respiratory distress syndrome (ARDS) and there are currently no targeted intervention strategies to treat or prevent it. The immune response to the virus is thought to both contribute to the pathogenesis of the disease and provide protection during its resolution. Thus, there is an unprecedented need to develop a safe and effective vaccine to immunize an extraordinarily large number of individuals.

### BRIEF SUMMARY OF THE INVENTION

The invention provides a recombinant virus comprising an influenza viral backbone, wherein the influenza viral backbone comprises PB1, PB2, PA, NP, M, NS, HA, and NA gene segments, wherein at least one of the PB1, PB2, PA, NP, M, NS, HA, and NA gene segments comprises at least one nucleotide sequence that encodes at least one antigen. In a preferred embodiment, the antigen is an immunogenic fragment of Severe Acute Respiratory Syndrome Coronavirus 2 (SARS-COV-2) spike glycoprotein.

### BRIEF DESCRIPTION OF THE SEVERAL VIEWS OF THE DRAWING(S)

FIG. 1 is a schematic of an influenza A NS segment engineered to express NS1 to SARS-COV-2 Spike receptor binding domain fusion protein. The construct includes a full length influenza A PR/8/1934 NS1 protein, a first linker (GSG1), amino acids 331-530 of SARS-COV-2 Wuhan-Hu-1 spike S1 protein encoding RBD (receptor binding domain), a second linker (GSG2), a cleavage site (P2A), and cDNA of essential PR8 nuclear export protein (NEP or NS2) with both Exon 1 and 2.

FIG. 2 is a schematic of an influenza A NS segment engineered to express SARS-CoV-2 Spike receptor binding domain as separate polypeptide. The construct includes a full length influenza A PR/8/1934 NS1 protein, a first linker (GSG1), a first cleavage site (T2A), amino acids 331-530 of SARS-COV-2 Wuhan-Hu-1 spike S1 protein encoding RBD, a second linker (GSG2), a second cleavage site (P2A), and cDNA of essential PR8 nuclear export protein (NEP or NS2) with both Exon 1 and 2.

FIG. 3 depicts an image of an immunoblot of cell lysates from Vero cells infected with CoV2 NS M2SR, M2SR control, and MOCK medium only. Proteins were separated by sodium dodecyl sulfate-polyacrylamide gel electrophoresis (SDS-PAGE) and subjected to immunoblot analysis. The primary antibody was anti-SARS-COV-2 RBD (Sino Biological Inc., Beijing, China) and the secondary antibody was an anti-rabbit IgG-horseradish peroxidase (HRP) with 3,3′,5,5′-tetramethylbenzidine (TMB) detection.

FIG. 4 is a set of images showing that both CoV2 NS1 M2SR and the standard M2SR infected cells express detectable levels of the influenza A NP protein. Meanwhile the FITC labeling of the RBD could only be detected in CoV2 NS1 M2SR infected cells providing significant detectable fluorescence.

FIG. 5 is a schematic of an influenza B M segment 7 engineered to express BM2 SARS-COV-2 Spike RBD fusion to amino and carboxy termini of BM2 protein (SEQ ID NOs: 84, 96). The construct includes a full length influenza B/Florida/4/2006 M1 protein, 5-mer translation stop/start site, amino acids 1-8 BM2 open reading frame (ORF), amino acids 330-524 of SARS-COV-2 Wuhan-Hu-1 spike S1 protein encoding RBD, and BM2 RBD fusion protein.

FIG. 6 is a schematic of an influenza B M segment 7 engineered to express BM2 SARS-COV-2 Spike RBD fusion to amino terminus of BM2 protein (SEQ ID NOs: 83, 95). The construct includes a full length influenza B/Florida/4/2006 M1 protein, 5-mer translation stop/start site, BM2 RBD fusion protein comprising amino acids 1-3 BM2 ORF, and 330-524 of SARS-COV-2 Wuhan-Hu-1 spike S1 protein encoding RBD.

FIG. 7 depicts an image of an immunoblot of cell lysates from Vero cells. Total cell lysates from cells infected with 2 SARS-COV-2 BM2SR strains (SEQ ID Nos: 83, 84, 95, 96) BM2SR, and MOCK medium only negative controls. Proteins were separated by SDS-PAGE and then subjected to immunoblot analysis. The primary antibody was anti-SARS-COV-2 RBD (Sino Biological Inc.) and the secondary antibody anti-rabbit IgG-HRP with TMB detection. Location of the RBD fusion protein is indicated in the image by pound signs.

FIG. 8A is a graph depicting mouse percent body weight change after immunization with the M2SR recombinant viruses.

FIG. 8B is a graph depicting mouse percent body weight change after immunization with the BM2SR recombinant viruses.

FIG. 9 is a bar graph showing the fold increase in enzyme-linked immunosorbent assay (ELISA) titer from pre-immunization baseline.

FIGS. 10A-10D are a set of graphs depicting the results of a study wherein mice (N=8) were immunized intranasally with monovalent H1N1 FGHY1-M2SR, monovalent H3N2 FGHY1-M2SR, bivalent H1N1 and H3N2 FGHY1-M2MR, monovalent BM2SR-Vic, monovalent BM2SR-Yam, bivalent BM2SR, trivalent H1N1 and H3N2 FGHY1-M2SR and BM2SR Victoria or Yamagata, or quadrivalent H1N1 and H3N2 FGHY1-M2SR and BM2SR Victoria and Yamagata vaccines or control (SPG). FIG. 10A depicts anti-H1 HA serum IgG ELISA titer data, FIG. 10B depicts anti-H3 HA data, FIG. 10C depicts anti-influenza B-Vic HA data, and FIG. 10D depicts data anti-influenza B-Yam HA.

FIG. 11 depicts a histogram of total cluster count versus number of hits in the cluster. Very few clusters had more than 10 hits as indicated by the grey shading between 10.0 and 20.0 hits.

FIG. 12 depicts a graph showing virus titer TCID₅₀ curves for two strains indicating that virus growth is not impaired by the synthetic segment expressing NS1 and NEP as a single self-cleaving peptide.

FIG. 13 depicts a graph showing a growth curve indicating that segment 8 with NS1 fusion to unmodified SARS-COV-2 helix antigen impairs the virus growth as compared to wild-type.

FIG. 14 is a schematic of an influenza A M segment 7 engineered to express SARS-CoV-2 Spike receptor binding domain fusion to amino terminus of M2 protein. The construct includes a full-length influenza A/PR/8/34 M1 protein, splice site, M2 RBD FLAG fusion protein comprising amino acids 1-25 M2 ORF, SARS-COV-2 MHC I compatible RBD antigen and FLAG tag, and stop codons.

FIG. 15 is a schematic of an influenza HA gene segment design for the creation of M2SR influenza virus capable of driving expression of antigen anchored to the extracellular membrane of infected cells. For FIGS. 15-20, “UTR” refers to “Untranslated Region,” “2A” refers to “2A self-cleaving peptide,” “MD” refers to “Multimerization Domain,” “TM” refers to “Transmembrane Domain,” and “ncr” refers to “Noncoding Region.”

FIG. 16 is a schematic of an influenza HA gene segment design for the creation of M2SR influenza virus capable of driving expression of antigen anchored to the extracellular membrane of infected cells.

FIG. 17 is a schematic of an influenza NS gene segment design for the creation of M2SR influenza virus capable of driving expression of antigen anchored to the extracellular membrane of infected cells.

FIG. 18 is a schematic of an influenza gene NS segment design for the creation of M2SR influenza virus capable of driving expression of antigen anchored to the extracellular membrane of infected cells.

FIG. 19 is a schematic of an influenza gene NA segment design for the creation of M2SR influenza virus capable of driving expression of antigen anchored to the extracellular membrane of infected cells.

FIG. 20 is a schematic of an influenza gene NA segment design for the creation of M2SR influenza virus capable of driving expression of antigen anchored to the extracellular membrane of infected cells.

FIG. 21 depicts the sequences of duplicated region encoding NS1 ORF and NEP Exon 1. Lower case letters indicate a mutation from A/PR/8/34. Bases 1-6 are deleted in the second copy of the NEP Exon 1 for NEP delta 2N mutant (SEQ ID NO: 110). The second copy of the duplicated region of NS segment encoding NEP Exon 1 is 63% identical to the first copy which is wild-type A/PR/8/34 NS segment cDNA sequence with a single nucleotide mutation that abolishes the splice donor site. (SEQ ID NO: 109).

FIG. 22 depicts the sequences of NS1 ORF and NEP Exon 2. Lower case letters indicate mutations from A/PR/8/34. The first copy of the duplicated region of the NS segment from NEP Exon 2 is 88% identical (SEQ ID NO: 111) to the second copy which is wild-type A/PR/8/34 NS segment cDNA sequence (SEQ ID NO: 112).

FIG. 23 depicts fluorescence micrograph images from 3 consecutive days post-inoculation of M2VeroA cells at MOI=10 by M2SR virus with engineered NS segment (SEQ ID NOs: 111, 112, and 114) expressing a tripartite polyprotein of NS1, EGFP and NEP peptides separated by T2A and P2A sites respectively (SEQ ID NO: 113).

FIG. 24 depicts flow cytometric analysis of immune stained live M2VeroA cells infected by M2SR vector virus only, or by M2SR virus with NS1 segment designed to direct expression of SARS-COV-2 S1 RBD mini-spike protein trimer at cell surface using SARS-COV-2 Spike signal sequence of only 12 amino acids, T4 Foldon and TM from RSV (SEQ ID NO: 115).

FIG. 25 depicts flow cytometric analysis of immune stained live M2VeroA cells infected by M2SR vector virus only, or by M2SR virus with HA segment with direct fusion of SARS-COV-2 S1 RBD to amino terminus of hemagglutinin from A/Singapore/2016 H3N2 influenza virus (SEQ ID NO: 116).

FIG. 26 depicts flow cytometric analysis of human 293T cells transfected with replicon DNA plasmid system with HA segment encoding direct fusion of respiratory syncytial virus surface glycoprotein G (RSV G) antigen to amino terminus of hemagglutinin from A/Singapore/2016 H3N2 influenza virus (SEQ ID NO: 117).

FIG. 27 depicts flow cytometric analysis of live M2VeroA cells infected by M2SR vector virus only, or by M2SR virus with NS1 segment designed to direct expression of SARS-CoV-2 mini-spike protein at cell surface using SARS-COV-2 S protein signal sequence, and S2 helical connector domain with SARS-COV-2 S protein TM (SEQ ID NO: 119).

FIG. 28 is a graph of average serum anti-SARS-COV2 RBD IgG titer of four dosing regimens pre-vaccination and post-prime and boost administration as described in Example 6.

### DETAILED DESCRIPTION OF THE INVENTION

The recombinant virus of the invention may be any type of virus. As used herein, a recombinant virus (e.g., a reassortant or different virus) is a virus comprising genetic material (e.g., gene segments) derived from a genetically distinct virus (e.g., heterologous gene segments).

As used herein, the term “gene segment” refers to the nucleotide sequence that encodes a viral protein. The gene segment may be represented by the cDNA (complementary DNA) sequence encoding the viral RNA (vRNA), i.e., SEQ ID NOs: 43-47, 53, 56, 58, 60, 63-67, and 73, that encodes the viral protein.

As used herein, the term “backbone” refers to the influenza gene segments encoding the PB1, PB2, PA, NP, NS1 and/or NS2, and M proteins. The gene segments of the invention encode proteins having selected amino acids. The viral backbone is an influenza viral backbone. There are four types of influenza viruses (i.e., A, B, C, and D) categorized based on their core proteins, although seasonal epidemics are most often caused by circulating influenza A and B viruses. In one embodiment, the influenza viral backbone is an influenza A backbone. In another embodiment, the influenza viral backbone is an influenza B backbone.

As used herein, the term “selected amino acid” refers to a specific amino acid in a particular position of an amino acid sequence. In some embodiments, the selected amino acid is the result of a genetic mutation to a parent amino acid sequence. The parent amino acid sequence may be identical to the amino acid sequence comprising the selected amino acid, except for the position corresponding to the selected amino acid.

### The Recombinant Virus

### (A) Influenza A Backbone Proteins

The PB1 (polymerase basic protein 1) gene segment of the invention may encode a protein, i.e., a PB1 protein, comprising at least one selected amino acid. In a preferred embodiment, the selected amino acids comprise a leucine at position 40 and a tryptophan at position 180. The selected amino acids of the PB1 protein further comprise at least one of an asparagine at position 464 or a serine at position 607. The PB1 gene segment may optionally comprise a cytosine to uracil promoter mutation at nucleotide position 4.

The selected amino acids may be acquired by genetic mutation to a parent PB1 sequence, e.g., a sequence identical to the PB1 amino acid sequence of the invention, except for the positions corresponding to the selected amino acids. The amino acid position 464 of the PB1 protein is located in the palm region of the influenza PB1 protein and connects RNA-dependent RNA polymerase activity domains. Generally, the aspartic acid at position 464 is highly conserved among influenza viruses isolated in eggs and in MDCK cells. Although the role of this amino acid has not been identified, the observed amino acid change to asparagine (N) at this position may affect PB1 protein conformation and may affect interaction with a host cell factor and, therefore, influenza polymerase activity in Vero cells. Moreover, influenza RNA polymerase is a heterotrimer composed of PA, PB1, and PB2 subunits. The histidine at position 465 of the PB1 protein interacts with glutamic acid at position 243 of the PA protein, and the amino acid change at position 464 of PB1 may alter interactions between PB1 and PA. The function of the amino acid at position 607 of the PB1 protein is also unknown; however, this amino acid is located between the RNA-dependent RNA polymerase region and the PB2 binding region, suggesting that it may alter interactions between PB1 and PB2, thereby affecting polymerase activity in Vero cells.

The PB2 (polymerase basic protein 2) gene segment of the invention may also encode a protein, i.e., a PB2 protein, comprising at least one selected amino acid. In a preferred embodiment, the selected amino acids comprise a valine at position 504 and optionally an isoleucine at position 467 and a valine at position 529. The PB2 gene segment may optionally comprise a cytosine to uracil promoter mutation at nucleotide position 4. The amino acids at position 467 and 529 of the PB2 protein are in the PB2-C portion. Specifically, the amino acid at potion 467 is located in the cap-binding region of the PB2 protein, and the amino acid at position 529 is located in the cap-627 linker domain. In some influenza viruses, the PB2 protein binds the cap structure of host capped RNA and utilizes the cap from the host RNA in order to make influenza mRNAs. This process is known as “cap-snatching.” Moreover, the amino acid at position 627 of PB2 is known to be a key determinant in host range and viral pathogenicity. Therefore, amino acid changes proximate to a cap-binding region may affect the efficiency of viral mRNA synthesis.

The PA (polymerase acidic protein) gene segment of the invention may also encode a protein, i.e., a PA protein, comprising at least one selected amino acid. In a preferred embodiment, the selected amino acids comprise a lysine at position 401. The PA gene segment may optionally comprise a cytosine to uracil promoter mutation at nucleotide position 4.

The NP (nucleoprotein) gene segment of the invention may also encode a protein, i.e., an NP protein, comprising at least one selected amino acid. In a preferred embodiment, the selected amino acids comprise a leucine at position 116 and at least one of a lysine at position 294 or an arginine at position 311. The amino acid positions 294 and 311 of the NP protein are located in the body of the NP protein, such that they neither serve as nuclear localization signals nor nuclear export signals.

The NS (non-structural) gene segment of the invention may also encode a protein, i.e., an NS1 and/or NS2 protein, comprising at least one selected amino acid. In a preferred embodiment, the selected amino acids comprise a proline at position 30 (NS1 protein) and a lysine at position 118 (NS1 protein).

In one embodiment of the invention, the influenza viral backbone comprises a PB1 gene segment encoding a protein, i.e., a PB1 protein, having selected amino acids at positions 40, 180, and 464, i.e., a leucine at position 40, a tryptophan at position 180, and an asparagine at position 464. The PB1 gene segment may have a nucleotide sequence represented by SEQ ID NO: 44. The PB1 gene segment may encode a protein, i.e., a PB1 protein, having an amino acid sequence of SEQ ID NO: 49. In another aspect of the embodiment, the influenza viral backbone may comprise a PB2 gene segment encoding a protein, i.e., a PB2 protein, having a selected amino acid at position 504, i.e., a valine at position 504. The PB2 gene segment may have a nucleotide sequence represented by SEQ ID NO: 56. The PB2 gene segment may encode a protein, i.e., a PB2 protein, having an amino acid sequence of SEQ ID NO: 57. The NP gene segment of the embodiment may encode a protein, i.e., an NP protein, having selected amino acids at positions 116 and 294, i.e., a leucine at position 116 and a lysine at position 294. The NP gene segment may have a nucleotide sequence represented by SEQ ID NO: 43. The NP gene segment may encode a protein, i.e., an NP protein, having an amino acid sequence of SEQ ID NO: 48. The PA and NS gene segments of the embodiment may also encode proteins, i.e., a PA protein and NS1 and/or NS2 protein, comprising selected amino acids at position 401 (PA protein), position 30 (NS1 protein), and position 118 (NS1 protein), i.e., a lysine at position 401 (PA protein), a proline at position 30 (NS1 protein), and a lysine at position 118 (NS1 protein). The PA gene segment may have a nucleotide sequence represented by SEQ ID NO: 58. The PA gene segment may encode a protein, i.e., a PA protein, having an amino acid sequence of SEQ ID NO: 59. The NS gene segment may have a nucleotide sequence represented by SEQ ID NO: 60. The NS gene segment may encode a protein, i.e., an NS1 protein, having an amino acid sequence of SEQ ID NO: 61. The NS gene segment may encode a protein, i.e., an NS2 protein, having an amino acid sequence of SEQ ID NO: 62. The PB1, PB2, and PA gene segments of the embodiment may also comprise a cytosine to uracil promoter mutation at nucleotide position 4.

In another embodiment of the invention, the influenza viral backbone comprises a PB1 gene segment encoding a protein, i.e., a PB1 protein, having selected amino acids at positions 40, 180, and 607, i.e., a leucine at position 40, a tryptophan at position 180, and a serine at position 607. The PB1 gene segment may have a nucleotide sequence represented by SEQ ID NO: 46. The PB1 gene segment may encode a protein, i.e., a PB1 protein, having an amino acid sequence of SEQ ID NO: 51. In another aspect of the embodiment, the influenza viral backbone may comprise a PB2 gene segment encoding a protein, i.e., PB2 protein, having selected amino acids at positions 504, 467, and 529, i.e., a valine at position 504, an isoleucine at position 467, and a valine at position 529. The PB2 gene segment may have a nucleotide sequence represented by SEQ ID NO: 47. The PB2 gene segment may encode a protein, i.e., a PB2 protein, having an amino acid sequence of SEQ ID NO: 52. The NP gene segment of the embodiment may encode a protein, i.e., an NP protein, having selected amino acids at positions 116 and 311, i.e., a leucine at position 116 and an arginine at position 311. The NP gene segment may have a nucleotide sequence represented by SEQ ID NO: 45. The NP gene segment may encode a protein, i.e., an NP protein, having an amino acid sequence of SEQ ID NO: 50. The PA and NS gene segments may also encode proteins, i.e., a PA protein and NS1 and/or NS2 protein, comprising selected amino acids at position 401 (PA protein), position 30 (NS1 protein), and position 118 (NS1 protein), i.e., a lysine at position 401 (PA protein), a proline at position 30 (NS1 protein), and a lysine at position 118 (NS1 protein). The PA gene segment may have a nucleotide sequence represented by SEQ ID NO: 58. The PA gene segment may encode a protein, i.e., a PA protein, having an amino acid sequence of SEQ ID NO: 59. The NS gene segment may have a nucleotide sequence represented by SEQ ID NO: 60. The NS gene segment may encode a protein, i.e., an NS1 protein, having an amino acid sequence of SEQ ID NO: 61. The NS gene segment may encode a protein, i.e., an NS2 protein, having an amino acid sequence of SEQ ID NO: 62. The PB1, PB2, and PA gene segments of the embodiment may also comprise a cytosine to uracil promoter mutation at nucleotide position 4.

The selected amino acids of the embodiments, particularly in most proteins of the backbone, confer enhanced growth properties onto the influenza viral backbone, as compared to an influenza viral backbone that is the same except without the selected amino acids, under the same conditions. For example, the influenza viral backbone of the invention exhibits enhanced growth in Vero cells.

The influenza viral backbone of the invention may also comprise an M (matrix protein) gene segment. In one embodiment of the invention, the M gene segment may be a mutant gene segment from influenza A, such that the virus lacks expression of functional M2 protein. Such a virus is herein referred to as an “M2SR” virus. As used herein, “M2SR” and “AM2SR” are interchangeable. The M2SR virus is a single replication influenza virus. The M gene segment of the M2SR virus may be represented by SEQ ID NO: 53. The M gene segment may encode a protein, e.g., a truncated M2 protein, having the amino acid sequence of SEQ ID NO: 54. The M2SR virus may be propagated in Vero cells that stably express the wild-type M2 protein (i.e., M2VeroA cells) to allow for multicycle replication. High yield in Vero cells is not dependent on mutation in the M gene segment. Therefore, the influenza viral backbone of the invention may comprise an M gene segment that encodes a functional M2 protein (SEQ ID NO: 1).

### (B) Influenza B Backbone Proteins

In one embodiment of the invention, the recombinant virus comprises an influenza viral backbone comprising PA, NP, and NS gene segments, wherein (a) the PA gene segment comprises a thymine at nucleotide position 2272; (b) the NP gene segment encodes a NP protein having an amino acid sequence comprising selected amino acids, wherein the selected amino acids comprise a serine at position 40, an asparagine or glycine at position 161, a threonine at position 204, and optionally a valine at position 93; and (c) the NS gene segment comprises a guanine at nucleotide position 39, and the NS gene segment encodes an NS protein having an amino acid sequence comprising selected amino acids, wherein the selected amino acids comprise a glutamine at position 176.

The PB1 (polymerase basic protein 1) gene segment of the invention may encode a protein, i.e., a PB1 protein, comprising at least one selected amino acid. The selected amino acids may be acquired by genetic mutation to a parent PB1 sequence, e.g., a sequence identical to the PB1 amino acid sequence of the invention, except for the positions corresponding to the selected amino acids. The PB2 (polymerase basic protein 2) gene segment of the invention may also encode a protein, i.e., a PB2 protein, comprising at least one selected amino acid.

The PA (polymerase acidic protein) gene segment of the invention may also encode a protein, i.e., a PA protein, comprising at least one selected amino acid. In a preferred embodiment, the gene segment comprises a thymine at nucleotide position 2272.

The NP (nucleoprotein) gene segment of the invention may also encode a protein, i.e., an NP protein, comprising at least one selected amino acid. In a preferred embodiment, the NP segment comprises a thymine at position 177, an adenine at position 540 and a thymine at position 670 and the NP gene segment encodes a protein having selected amino acids comprise a serine at position 40, an asparagine or glycine at position 161, a threonine at position 204, and optionally a valine at position 93.

The NS (non-structural) gene segment of the invention may also encode a protein, i.e., an NS1 and/or NS2 protein, comprising at least one selected amino acid. In a preferred embodiment, the NS segment comprises a guanine at nucleotide position 39 and a cytosine at position 570 and the NS gene segment encodes an NS protein having selected amino acids comprising a glutamine at position 176 (NS1 protein).

In one embodiment of the invention, the influenza virus comprises a PB1 gene segment encoding a protein, i.e., a PB1 protein, having selected amino acids. The PB1 gene segment may have a nucleotide sequence represented by SEQ ID NO: 63. The PB1 gene segment may encode a protein, i.e., a PB1 protein, having an amino acid sequence of SEQ ID NO: 68. In another aspect of the embodiment, the influenza virus may comprise a PB2 gene segment encoding a protein, i.e., a PB2 protein, having selected amino acids. The PB2 gene segment may have a nucleotide sequence represented by SEQ ID NO: 64. The PB2 gene segment may encode a protein, i.e., a PB2 protein, having an amino acid sequence of SEQ ID NO: 69. In another aspect of the embodiment, the influenza virus may comprise a NP gene segment encoding a protein, i.e., a NP protein, having selected amino acids at positions 40, 161, and 204, i.e., a serine at position 40, an asparagine or glycine at position 161, a threonine at position 204, and optionally a valine at position 93. The NP gene segment may have a nucleotide sequence represented by SEQ ID NO: 66. The NP gene segment may encode a protein, i.e., an NP protein, having an amino acid sequence of SEQ ID NO: 71. In another aspect of the embodiment, the influenza virus may comprise a NS gene segment encoding a protein, i.e., a NS1 and/or NS2 protein, having selected amino acids at position 176, i.e., a glutamine at position 176. The NS gene segment may comprise a guanine at nucleotide position 39 and cytosine at position 570. The NS gene segment may have a nucleotide sequence represented by SEQ ID NO: 67. The NS gene segment may encode a protein, i.e., an NS1 and/or NS2 protein, having an amino acid sequence of SEQ ID NO: 72. In another aspect of the embodiment, the influenza virus may comprise a PA gene segment encoding a protein, i.e., a PA protein. The PA gene segment may have a nucleotide sequence represented by SEQ ID NO: 65. The PA gene segment may encode a protein, i.e., a PA protein, having an amino acid sequence of SEQ ID NO: 70.

The selected amino acids of the embodiments, particularly in most proteins of the backbone, confer enhanced growth properties onto the influenza virus, as compared to an influenza virus that is the same except without the selected amino acids, under the same conditions. For example, the influenza virus of the invention exhibits enhanced growth in Vero cells.

The influenza virus of the invention may also comprise an M (matrix protein) gene segment. In one embodiment of the invention, the M gene segment may be a mutant gene segment from influenza B, such that the virus lacks expression of functional BM2 protein. Such a virus is herein referred to as a “BM2SR” virus. The BM2SR virus is a single replication influenza virus. The M gene segment of the BM2SR virus may be represented by SEQ ID NO: 73. The M gene segment may encode a protein, e.g., a truncated BM2 protein, having the amino acid sequence of SEQ ID NO: 78. The BM2SR virus may be propagated in Vero cells that stably express the BM2 protein (i.e., BM2VeroA cells) to allow for multicycle replication. High yield in Vero cells is not dependent on mutation in the M gene segment. Therefore, the influenza virus of the invention may comprise an M gene segment that encodes a functional BM2 protein (SEQ ID NO: 2).

### (C) Influenza A Surface Proteins

In a further embodiment of the invention, the influenza viral backbone comprises an NA (neuraminidase) and HA (hemagglutinin) gene segment. In one embodiment of the invention, the HA gene segment may encode an HA protein having an amino acid sequence comprising at least one selected amino acid (e.g., an amino acid mutation) in the HA1 subunit of the protein and/or at least one selected amino acid (e.g., amino acid mutation) in the HA2 subunit of the protein. For example, the at least one amino acid mutation in the HA2 subunit may be an asparagine at position 107. Such mutations may also contribute to enhanced growth of the virus during production.

In one embodiment of the invention, the PB1, PB2, PA, NP, and NS gene segments are derived from a single influenza strain. The HA gene segment may be derived from an influenza strain different from the single influenza strain from which the PB1, PB2, PA, NP, and NS gene segments are derived. Likewise, the NA gene segment may be derived from an influenza strain different from the single influenza strain from which the PB1, PB2, PA, NP, and NS gene segments are derived. Accordingly, the recombinant virus of the invention may be a pandemic virus (e.g., H5N1 and H7N9) or a seasonal virus (e.g., H1N1, H3N2, and influenza B).

### (D) Influenza B Surface Proteins

In a further embodiment of the invention, the recombinant virus comprises an influenza viral backbone further comprising an NA (neuraminidase) and HA (hemagglutinin) gene segment. In one embodiment of the invention, the HA gene segment may encode a HA protein having an amino acid sequence comprising at least one selected amino acid (e.g., an amino acid mutation) in the HA1 subunit of the protein and/or at least one selected amino acid (e.g., amino acid mutation) in the HA2 subunit of the protein. For example, the at least one amino acid mutation in the HA2 subunit may be a glutamic acid at position 61. In another embodiment, the at least one amino acid mutation in the HA2 subunit may be glutamic acid at position 112. The amino acid mutations may be present in any of the subtypes or lineages of influenza B virus (i.e., Victoria or Yamagata). In a preferred embodiment, the amino acid mutation in the HA2 subunit may be glutamic acid at position 61 in the Victoria lineage of influenza B virus. In another preferred embodiment, the amino acid mutation in the HA2 subunit may be glutamic acid at position 112 in the Yamagata lineage of the influenza B virus. Such mutations may also contribute to enhanced growth of the virus during production.

In one embodiment of the invention, the PB1, PB2, PA, NP, and NS gene segments are derived from a single influenza strain. The HA gene segment may be derived from an influenza strain different from the single influenza strain from which the PB1, PB2, PA, NP, and NS gene segments are derived. Likewise, the NA gene segment may be derived from an influenza strain different from the single influenza strain from which the PB1, PB2, PA, NP, and NS gene segments are derived. Accordingly, the influenza virus of the invention may be a seasonal influenza virus (e.g., influenza B).

### (E) Antigens

In one embodiment, the recombinant virus comprises an influenza viral backbone comprising PB1, PB2, PA, NP, M, NS, HA, and NA gene segments, at least one of the PB2, PB2, PA, NP, M, NS, HA, and NA gene segments comprises a nucleotide sequence that encodes one or more antigens. As used herein, the term “antigen” refers to an antigen heterologous with respect to the HA gene segment. The antigen can be viral (including influenza), bacterial, fungal, or protozoal. For example, a viral antigen or epitope sequence that is inserted into a gene segment (e.g., PB1, PB2, PA, NP, M, NS, HA, or NA gene segments) would be an antigen as to the virus. In one embodiment, the antigen is an immunogenic fragment of SARS-COV-2 spike glycoprotein (e.g., S1 protein). In another embodiment, the antigen is an influenza gene segment or fragment thereof (i.e., PB1, PB2, PA, NP, M, NS, HA, or NA gene segments or fragments thereof) that is heterologous to the HA gene segment in the influenza viral backbone. In another embodiment the antigen is respiratory syncytial virus (RSV) or a fragment thereof. In another embodiment, the antigen is parainfluenza virus (PIV) or a fragment thereof. In some embodiments, the one or more antigens are expressed from within a viral gene segment.

In one embodiment of the invention, at least one gene segment that comprises a nucleotide sequence that encodes one or more antigens further comprises a nucleotide sequence that encodes at least one flexible linker protein, at least one cleavable cleavage sequence, and/or at least one FLAG protein. Such a gene segment may encode at least two flexible linker proteins, at least two cleavable cleavage sequences, and/or at least two FLAG proteins.

In an embodiment of the invention, the cleavable cleavage sequence comprises a “self cleaving” sequence. In an embodiment, the “self cleaving” sequence is a “self cleaving” 2A peptide. In an embodiment, the “self cleaving” sequence is a “self cleaving” 2A peptide. “Self cleaving” 2A peptides are described, for example, in Liu et al., Sci. Rep., 7(1): 2193 (2017), and Szymczak et al., Nature Biotechnol., 22(5): 589-594 (2004). The 2A peptides are viral oligopeptides that mediate cleavage of polypeptides during translation in eukaryotic cells. The designation “2A” refers to a specific region of the viral genome. Without being bound to a particular theory or mechanism, it is believed that the mechanism of 2A-mediated “self cleavage” is ribosome skipping of the formation of a glycyl-prolyl peptide bond at the C-terminus of the 2A peptide. Different 2A peptides may comprise, at the C-terminus, the consensus amino acid sequence of GDVEXNPGP (SEQ ID NO: 19), wherein X of SEQ ID NO: 19 is any naturally occurring amino acid residue. In an embodiment of the invention, the cleavable ribosomal skip sequence is a porcine teschovirus-1 2A (P2A) amino acid sequence, equine rhinitis A virus (E2A) amino acid sequence, thosea asigna virus 2A (T2A) amino acid sequence, or foot-and-mouth disease virus (F2A) amino acid sequence. In an embodiment of the invention, the ribosomal skip sequence is a 2A peptide amino acid sequence comprising, consisting, or consisting essentially of, the amino acid sequence of P2A.

In an embodiment of the invention, the flexible linker protein is 1 to 20 amino acid residues selected, independently, from the group consisting of glycine and serine. In some embodiments, the flexible linker protein is defined as (Xaa1)ᵣ, wherein each Xaa1 is selected independently from glycine and serine and r is an integer from 1 to 20. An example of such linker includes, but is not limited to GSG (SEQ ID NO: 75), GGGGSGGGGSGGGGS (SEQ ID NO: 76), and (G4S)₃. In one embodiment of the invention the at least one gene segment that comprises an amino acid sequence that encodes an antigen further comprises at least one flexible linker proteins. In another embodiment, such a gene segment further comprises at least two flexible linker proteins.

In a preferred embodiment, the antigen is an immunogenic fragment of SARS-COV-2 spike glycoprotein (e.g., S1 protein). In one embodiment, the M gene segment encodes a nucleotide sequence encoding at least one immunogenic fragment of SARS-COV-2 spike glycoprotein. The M gene segment may encode a mutated M2 or BM2 protein. The M gene segment may further encode at least one flexible linker protein and at least one FLAG protein. In one embodiment, the M gene segment encodes a fusion protein comprising a mutated M2 protein, a flexible linker protein and a FLAG epitope tag protein. For example, the M gene segment may have a nucleotide sequence represented by any one of SEQ ID NOs: 79 and 81-84. The M gene segment may encode a protein comprising any one of SEQ ID NOs: 1-14 and 92-96.

In another embodiment, the NS gene segment encodes a nucleotide sequence encoding at least one immunogenic fragment of SARS-COV-2 spike glycoprotein. The NS gene segment may encode a NS1 protein and NS2 (i.e., NEP) protein or fragment thereof. The NS gene segment may also encode at least one flexible linker protein or fragment thereof. The NS gene segment may also encode at least one cleavable cleavage sequence. The NS gene segment may have a nucleotide sequence represented by any one of SEQ ID NOs: 80 and 85-91. The NS gene segment may encode a protein comprising SEQ ID NOs: 97-104.

In one embodiment of the invention, the cleavable cleavage sequence is a P2A peptide sequence. In such an embodiment, the P2A peptide sequence is bound to the C-terminus of the NS1 protein on one end and the antigen on the other. In such an embodiment, the antigen can be bound to the NEP Open Reading Frame (ORF). In another embodiment of the invention, the P2A peptide sequence is bound to the C-terminus of the NS1 protein on one end and a first flexible linker protein on the other end. In such an embodiment, a first flexible linker protein can be bound to the antigen, which is attached to the NEP ORF. In another embodiment, a second cleavable cleavage sequence is present. In one embodiment, the second cleavable cleavage sequence is a P2A or T2A peptide sequence. The optional second cleavable cleavage sequence may be bound to the antigen on one end and the NEP ORF on the other end. In another embodiment, the second cleavable cleavage sequence may be bound to a flexible linker protein, which is then bound to either the antigen or the NEP ORF.

In one embodiment at least one (i.e., PB2, PB2, PA, NP, M, NS, HA, or NA) gene segment will encode a nucleotide sequence that encodes one or more antigens. In some embodiments, at least two (i.e., PB1 and PB2, PB1 and PA, PB1 and NP, PB1 and M, PB1 and NS, PB1 and HA, and PB1 and NA, PB2 and PA, PB2 and NP, PB2 and M, PB2 and NS, PB2 and HA, PB2 and NA, PA and NP, PA and M, PA and NS, PA and HA, PA and NA, NP and M, NP and NS, NP and HA, NP and NA, M and NS, M and HA, M and NA, NS and HA, NS and NA, or HA and NA gene segments) of the eight influenza viral backbone segments will encode a nucleotide sequence that encodes one or more antigens, such as an immunogenic fragment of SARS-COV-2 spike glycoprotein (e.g., S1 protein)).

In some embodiments, the gene segment that comprises at least one nucleotide sequence that encodes one or more antigens further comprises a downstream duplication, wherein the downstream duplication comprises at least one silent nucleotide mutation. In one embodiment, the gene segment that comprises at least one nucleotide sequence that encodes one or more antigens further comprises a downstream direct tandem duplication, wherein the downstream duplication comprises at least one silent nucleotide mutation. A downstream duplication refers to a nucleotide sequence in which a portion of the nucleotide sequence is repeated one or more times in the same orientation. The repeat nucleotide sequences can be lined up one directly after another, or they can contain optional nucleotide sequences between each of the repeat nucleotide sequences. In addition, the number of duplicated bases is not limited.

In some embodiments, a downstream duplication of a nucleotide sequence of the gene segment occurs during insertion of the nucleotide sequence encoding an antigen. In some embodiments, the downstream duplication can reduce the stability of the nucleotide sequence and the encoded amino acid sequences and proteins. To improve stability, at least one silent mutation (i.e., a mutation that does not affect the amino acid sequence encoded by the nucleotide sequence) is introduced to reduce the homology between a first nucleotide sequence and the second downstream duplicated nucleotide sequence. For example, in a preferred embodiment, the NS gene segment comprises a nucleotide sequence encoding an antigen. During the insertion of the nucleotide sequence encoding an antigen, a portion of the nucleotide sequence is duplicated, creating a downstream duplication. The first copy of the nucleotide sequence is part of the packaging sequence, and the second copy can interfere with packaging. To prevent interference, silent mutations are added to the downstream duplication to reduce homology with the first copy. In one embodiment, the downstream duplication has at least one (i.e., at least one, at least two at least three, at least four, at least five, at least six, at least seven, at least eight, at least nine, or at least ten) silent mutation(s).

The recombinant virus may have one or more (i.e., at least two, at least three, at least four, at least five, at least six, at least seven, or at least eight) gene segments that comprise at least one nucleotide sequence that encodes one or more antigens and further comprise a downstream duplication, wherein the downstream duplication comprises at least one silent nucleotide mutation. For example, in one embodiment, such one or more gene segments can be the PB1, PB2, PA, NP, NS, M, HA, or NA gene segments. In another embodiment, such one or more gene segments can be the PB1 and PB2, PB2 and PA, PB1 and NP, PB1 and NS, PB1 and M, PB1 and HA, PB1 and NA, PB2 and PA, PB2 and NP, PB2 and NS, PB2 and M, PB2 and HA, PB2 and NA, PA and NP, PA and NS, PA and M, PA and HA, PA and NA, NP and NS, NP and M, NP and HA, NP and NA, NS and M, NS and HA, NS and NA, M and HA, M and NA, or HA and NA gene segments.

### (F) Properties of the Influenza Viral Backbone

The backbone of the inventive recombinant virus confers high growth properties onto influenza viruses, particularly in Vero cells, regardless of the type of influenza virus (e.g., influenza A or B, seasonal or pandemic influenza viruses). The inventive influenza virus exhibits high yields even in manufacturing processes using low multiplicity of infection (MOI) (e.g., 0.001). MOI refers to the average number of agent (e.g., virus) per infection target (e.g., cell). A lower MOI is used when multiple cycles of infection are required (e.g., virus vaccine production). Current Good Manufacturing Practice regulations are enforced by the US FDA and generally necessitate use of the lowest MOI that still produces high yields of the virus. This is because master seed stocks are costly, and toxicity resulting from noninfectious particles and excess cellular proteins can decrease virus production.

In a further embodiment of the invention, the influenza virus is genetically stable, such that the selected amino acids of the backbone proteins, particularly the PB1, PB2, PA, NP, and NS1 proteins, are highly conserved, even when propagated at low MOI. For example, in one embodiment of the invention, the selected amino acids are conserved in at least one of the PB1, PB2, and NP proteins after at least one, at least two, at least three, at least four, at least five, at least six, at least seven, at least eight, at least nine, at least ten, or more than ten serial passages in a Vero cell line. In one embodiment, the Vero cell line may comprise Vero cells that stably express the M2 ion channel protein of influenza A virus (i.e., M2VeroA cells). In another embodiment of the invention, the Vero cell line may comprise Vero cells that stably express the BM2 ion channel protein of influenza B virus (SEQ ID NO: 74) (i.e., BM2Vero cells). BM2 is known to be a functional counterpart to influenza A virus M2. Influenza B virus M2 protein can functionally replace its influenza A virus counterpart in promoting virus replication (Wanitchang et al., Virology 498:99-108 (2016)). In such an embodiment, the selected amino acids may be conserved even when the influenza virus is an influenza A virus.

Genetically modified Vero cells (i.e., those that express influenza M2 or BM2 proteins) behave like normal Vero cells and support growth of influenza A or B viruses comparable to normal Vero cells. Virus titers for M2SR viruses in M2VeroA cells are comparable to replicating influenza viruses that express functional M2 in unmodified Vero cell lines. Further, virus titers for BM2SR viruses (i.e., influenza viruses that comprise a mutant M gene segment from influenza B and consequently do not express a functional BM2 protein) in BM2Vero cells are comparable to replicating influenza viruses that express functional BM2 in unmodified Vero cell lines. Accordingly, M2SR and BM2SR viruses behave like replicating influenza viruses in the M2VeroA and BM2Vero cell lines.

In one embodiment of the invention, the influenza virus is capable of replication in human cells.

### Pharmaceutical Formulation

The invention provides a pharmaceutical formulation (e.g., a vaccine or other immunogenic composition) comprising the inventive recombinant virus as described herein.

The pharmaceutical formulation can further comprise at least one pharmaceutically acceptable carrier or excipient. As used herein, the term “pharmaceutically acceptable carrier or excipient” refers to any component of the pharmaceutical formulation other than the inventive influenza virus. The pharmaceutically acceptable carrier or excipient can enhance efficacy of the inventive recombinant virus or maintain stability of the pharmaceutical formulation, desirably without significantly inactivating the inventive recombinant virus.

The at least one pharmaceutically acceptable carrier or excipient may be any suitable pharmaceutically acceptable carrier or excipient, many of which are known in the art. Exemplary pharmaceutically acceptable carriers or excipients include components that maintain a pH of the pharmaceutical formulation (e.g., buffers), adjust tonicity (e.g., tonicity modifying agents such as an inorganic salt), improve protein (e.g., virus) stability and/or immunogenicity, improve mucoadhesion, prevent protein aggregation, and/or preserve the pharmaceutical formulation (e.g., preservatives). For example, the pharmaceutically acceptable carrier or excipient may comprise at least one of an inorganic salt, surfactant, amino acid, polymer or polymeric compound (e.g., protein, polysaccharide, or hydrogel), chelating agent, sugar, polyol, and/or adjuvant (e.g., any substance that augments a specific immune response), many of which are known in the art. A particular carrier or excipient may serve more than one purpose in the pharmaceutical formulation, and, thus, the following embodiments are not limited to the descriptions recited herein.

Any suitable buffer can be present in the pharmaceutical formulation. In one embodiment, the buffer comprises at least one of an imidazole buffer, a potassium phosphate buffer, phosphate-buffered saline (PBS), Dulbecco's phosphate-buffered saline (DPBS) (e.g., 1×DPBS), a histidine buffer, a sodium citrate buffer, and sucrose phosphate glutamate buffer (SPG). PBS and/or DPBS preparations may comprise, for example, sodium chloride, potassium chloride, potassium phosphate monobasic, and sodium phosphate dibasic, and may optionally further comprise calcium chloride and/or magnesium chloride. In some embodiments, the PBS and/or DPBS preparations comprise about 136.9 mM sodium chloride, about 2.67 mM potassium chloride, about 1.47 mM potassium phosphate monobasic, and about 8.1 mM sodium phosphate dibasic, although any suitable PBS and/or DPBS preparation, many of which are known in the art, may be used as a buffer in the pharmaceutical formulation.

The buffer can be present in the pharmaceutical formulation in any suitable concentration. The buffer can be present in the pharmaceutical formulation at a concentration of about 0.1 mM or more, about 1 mM or more, about 10 mM or more, about 20 mM or more, about 30 mM or more, about 40 mM or more, about 50 mM or more, about 60 mM or more, about 70 mM or more, about 80 mM or more, about 90 mM or more, about 100 mM or more, about 120 mM or more, about 140 mM or more, about 160 mM or more, about 180 mM or more, about 200 mM or more, about 250 mM or more, about 300 mM or more, about 350 mM or more, about 400 mM or more, about 450 mM or more, or about 500 mM or more. Alternatively, or in addition, the buffer can be present in the pharmaceutical formulation at a concentration of about 1,000 mM or less, about 500 mM or less, about 450 mM or less, about 400 mM or less, about 350 mM or less, about 300 mM or less, about 250 mM or less, about 200 mM or less, about 180 mM or less, about 160 mM or less, about 140 mM or less, about 120 mM or less, about 100 mM or less, about 90 mM or less, about 80 mM or less, about 70 mM or less, about 60 mM or less, about 50 mM or less, about 40 mM or less, about 30 mM or less, about 20 mM or less, about 10 mM or less, or about 1 mM or less. The buffer can be present in the pharmaceutical formulation at any concentration within a range bounded by any of the aforementioned endpoints. For example, the buffer can be present in the pharmaceutical formulation at a concentration of about 0.1 mM to about 1000 mM, about 0.1 mM to about 500 mM, about 0.1 mM to about 100 mM, about 1 mM to about 1000 mM, about 1 mM to about 500 mM, about 1 mM to about 100 mM, about 100 mM to about 1000 mM, about 100 mM to about 500 mM, and the like.

In further embodiments, the buffer is present in the pharmaceutical formulation at a percentage concentration (e.g., volume/volume percentage (% v/v); weight/volume percentage (% w/v); or weight/weight percentage (% w/w)). The buffer can be present in the pharmaceutical formulation at a percentage concentration of about 0.1% or more, about 1% or more, about 5% or more, about 10% or more, about 15% or more, about 20% or more, about 30% or more, about 40% or more, or about 50% or more. Alternatively, or in addition, the buffer can be present in the pharmaceutical formulation at a percentage concentration of about 60% or less, about 50% or less, about 40% or less, about 30% or less, about 20% or less, about 15% or less, about 10% or less, about 5% or less, or about 1% or less. The buffer can be present in the pharmaceutical formulation at any percentage concentration within a range bounded by any of the foregoing endpoints. For example, the buffer can be present in the pharmaceutical formulation at a percentage concentration of about 0.1% to about 60%, about 1% to about 60%, about 10% to about 60%, about 0.1% to about 50%, about 1% to about 50%, about 10% to about 50%, about 20% to about 60%, about 20% to about 50%, about 20% to about 40%, about 20% to about 30%, about 30% to about 40%, about 40% to about 50%, and the like.

The buffer can maintain the pH of the pharmaceutical formulation at any suitable pH. The buffer can maintain the pH of the pharmaceutical formulation at a pH of, for example, about 4 or higher, about 4.5 or higher, about 5 or higher, about 5.5 or higher, about 6 or higher, about 6.5 or higher, about 7 or higher, or about 7.5 or higher. Alternatively, or in addition, the buffer can maintain the pH of the pharmaceutical formulation at a pH of, for example, about 8 or lower, about 7.5 or lower, about 7 or lower, about 6.5 or lower, about 6 or lower, about 5.5 or lower, about 5 or lower, or about 4.5 or lower. The buffer can maintain the pH of the pharmaceutical formulation at a pH within a range bounded by any of the foregoing endpoints. For example, the buffer can maintain the pH of the pharmaceutical formulation at a pH of about 4 to about 8, about 4.5 to about 8, about 5 to about 8, about 5.5 to about 8, about 6 to about 8, about 6.5 to about 8, about 7 to about 8, about 7.5 to about 8, about 4 to about 7.5, about 5 to about 7.5, about 6 to about 7.5, about 7 to about 7.5, about 4 to about 7, about 5 to about 7, about 6 to about 7, and the like.

Any suitable tonicity modifying agent can be present in the pharmaceutical formulation. In certain embodiments, one or more inorganic salts are present in the pharmaceutical formulation as tonicity modifying agents. The inorganic salt(s) may be at least one of sodium chloride (NaCl), magnesium sulfate (MgSO₄), and magnesium chloride (MgCl₂). The tonicity modifying agent, e.g., inorganic salt(s), can be present in the pharmaceutical formulation in any suitable amount. The tonicity modifying agent, e.g., inorganic salt(s), can be present in the pharmaceutical formulation at a concentration of about 0.1 mM or more, about 0.2 mM or more, about 0.4 mM or more, about 0.6 mM or more, about 0.8 mM or more, about 1 mM or more, about 1.2 mM or more, about 1.4 mM or more about 1.6 mM or more, about 1.8 mM or more, about 2 mM or more, about 3 mM or more, about 4 mM or more, about 5 mM or more, about 6 mM or more, about 7 mM or more, about 8 mM or more, about 9 mM or more, about 10 mM or more, about 20 mM or more, about 30 mM or more, about 40 mM or more, about 50 mM or more, about 100 mM or more, about 200 mM or more, about 300 mM or more, about 400 mM or more, about 500 mM or more, about 600 mM or more, about 700 mM or more, about 800 mM or more, about 900 mM or more, about 1000 mM or more, or about 1500 mM or more. Alternatively, or in addition, the tonicity modifying agent, e.g., inorganic salt(s), can be present in the pharmaceutical formulation at a concentration of about 2000 mM or less, about 1500 mM or less, about 1000 mM or less, about 900 mM or less, about 800 mM or less, about 700 mM or less, about 600 mM or less, about 500 mM or less, about 450 mM or less, about 400 mM or less, about 350 mM or less, about 300 mM or less, about 250 mM or less, about 200 mM or less, about 150 mM or less, about 100 mM or less, about 50 mM or less, about 45 mM or less, about 40 mM or less, about 35 mM or less, about 30 mM or less, about 25 mM or less, about 20 mM or less, about 10 mM or less, about 9 mM or less, about 8 mM or less, about 7 mM or less, about 6 mM or less, about 5 mM or less, about 4 mM or less, about 3 mM or less, about 2 mM or less, about 1.8 mM or less, about 1.6 mM or less, about 1.4 mM or less, about 1.2 mM or less, about 1 mM or less, about 0.8 mM or less, about 0.6 mM or less, about 0.4 mM or less, or about 0.2 mM or less. The tonicity modifying agent, e.g., inorganic salt(s), can be present in the pharmaceutical formulation at any concentration within a range bounded by any of the aforementioned endpoints. For example, the tonicity modifying agent, e.g., inorganic salt(s), can be present in the pharmaceutical formulation at a concentration of about 0.1 mM to about 2000 mM, about 0.1 mM to about 1500 mM, about 0.1 mM to about 1000 mM, about 0.1 mM to about 500 mM, about 0.1 mM to about 250 mM, about 0.1 mM to about 100 mM, about 0.1 to about 50 mM, about 0.1 mM to about 10 mM, about 1 mM to about 2000 mM, about 1 mM to about 1500 mM, about 1 mM to about 1000 mM, about 1 mM to about 500 mM, about 1 mM to about 250 mM, about 1 mM to about 100 mM, about 1 mM to about 50 mM, about 1 mM to about 10 mM, about 10 mM to about 2000 mM, about 10 mM to about 1500 mM, about 10 mM to about 1000 mM, about 10 mM to about 500 mM, about 10 mM to about 250 mM, about 10 mM to about 100 mM, about 10 mM to about 50 mM, about 100 mM to about 2000 mM, about 100 mM to about 1500 mM, about 100 mM to about 1000 mM, about 100 mM to about 500 mM, about 100 mM to about 250 mM, about 500 mM to about 2000 mM, about 500 mM to about 1500 mM, about 500 mM to about 1000 mM, and the like.

In further embodiments, the inorganic salt is present in the pharmaceutical formulation at a percentage concentration (e.g., volume/volume percentage (% v/v); weight/volume percentage (% w/v); or weight/weight percentage (% w/w)). The tonicity modifying agent, e.g., inorganic salt(s), be present in the pharmaceutical formulation at a percentage concentration of about 0.1% or more, about 1% or more, about 2% or more, about 3% or more, about 4% or more, about 5% or more, about 6% or more, about 7% or more, about 8% or more, about 9% or more, or about 10% or more. Alternatively, or in addition, the tonicity modifying agent, e.g., inorganic salt(s), can be present in the pharmaceutical formulation at a percentage concentration of about 10% or less, about 9% or less, about 8% or less, about 7% or less, about 6% or less, about 5% or less, about 4% or less, about 3% or less, about 2% or less, or about 1% or less. The tonicity modifying agent, e.g., inorganic salt(s), can be present in the pharmaceutical formulation at any percentage concentration within a range bounded by any of the foregoing endpoints. For example, the tonicity modifying agent, e.g., inorganic salt(s), can be present in the pharmaceutical formulation at a percentage concentration of about 0.1% to about 1%, about 0.1% to about 2%, about 0.1% to about 5%, about 0.1% to about 10%, about 1% to about 2%, about 1% to about 5%, about 1% to about 10%, about 2% to about 10%, about 3% to about 10%, about 4% to about 10%, about 5% to about 10%, and the like.

Any suitable surfactant can be present in the pharmaceutical formulation. In certain embodiments, the surfactant can comprise at least one of polysorbate 20, polysorbate 80, sodium deoxycholate, and poloxamer 188. The surfactant can be present in the pharmaceutical formulation in any suitable amount. In some embodiments, the surfactant is present in the pharmaceutical formulation at a percent concentration (e.g., volume/volume percentage (% v/v); weight/volume percentage (% w/v); or weight/weight percentage (% w/w)). The surfactant can be present in the pharmaceutical formulation at a percentage concentration of about 0.01% or more, about 0.02% or more, about 0.03% or more, about 0.04% or more, about 0.05% or more, about 0.06% or more, about 0.07% or more, about 0.08% or more, about 0.09% or more, about 0.1% or more, about 0.2% or more, about 0.3% or more, about 0.4% or more, about 0.5% or more, about 0.6% or more, about 0.7% or more, about 0.8% or more, about 0.9% or more, or about 1% or more. Alternatively, or in addition, the surfactant can be present in the pharmaceutical formulation at a percentage concentration of about 1% or less, about 0.9% or less, about 0.8% or less, about 0.7% or less, about 0.6% or less, about 0.5% or less, about 0.4% or less, about 0.3% or less, about 0.2% or less, or about 0.1% or less. The surfactant can be present in the pharmaceutical formulation at any percentage concentration within a range bounded by any of the foregoing endpoints. For example, the surfactant can be present in the pharmaceutical formulation at a percentage concentration of about 0.01% to about 1%, about 0.01% to about 0.1%, about 0.05% to about 1%, about 0.05% to about 0.1%, about 0.1% to about 1%, about 0.1% to about 0.5%, about 0.2% to about 1%, about 0.5% to about 1%, and the like.

Any suitable amino acids can be present in the pharmaceutical formulation. In certain embodiments, the amino acid may be one or more of arginine, glutamic acid or glutamate, asparagine, histidine, and glycine. The amino acid(s) can be present in the pharmaceutical formulation in any suitable amount. The amino acid(s) can be present in the pharmaceutical formulation at a concentration of about 1 mM or more, about 2 mM or more, about 3 mM or more, about 5 mM or more, about 6 mM or more, about 7 mM or more, about 8 mM or more, about 9 mM or more, or about 10 mM or more. Alternatively, or in addition, the amino acid(s) can be present in the pharmaceutical formulation at a concentration of about about 100 mM or less, about 90 mM or less, about 80 mM or less, about 70 mM or less, about 60 mM or less, about 50 mM or less, about 40 mM or less, about 30 mM or less, about 20 mM or less, or about 10 mM or less. The amino acid(s) can be present in the pharmaceutical formulation at any concentration within a range bounded by any of the foregoing endpoints. For example, the amino acid(s) can be present in the pharmaceutical formulation at a concentration of about 1 mM to about 10 mM, about 1 mM to about 50 mM, about 1 mM to about 100 mM, about 5 mM to about 50 mM, about 10 mM to about 50 mM, about 20 mM to about 50 mM, and the like.

In some embodiments, the amino acid(s) is present in the pharmaceutical formulation at a percentage concentration (e.g., volume/volume percentage (% v/v); weight/volume percentage (% w/v); or weight/weight percentage (% w/w)). The amino acid(s) can be present in the pharmaceutical formulation at a percentage concentration of about 0.1% or more, about 0.2% or more, about 0.3% or more, about 0.4% or more, about 0.5% or more, about 0.6% or more, about 0.7% or more, about 0.8% or more, about 0.9% or more, about 1% or more, about 2% or more, about 3% or more, about 4% or more, or about 5% or more. Alternatively, or in addition, the amino acid(s) can be present in the pharmaceutical formulation at a percentage concentration of about 10% or less, about 9% or less, about 8% or less, about 7% or less, about 6% or less, about 5% or less, about 4% or less, about 3% or less, about 2% or less, or about 1% or less. The amino acid(s) can be present in the pharmaceutical formulation at any percentage concentration within a range bounded by any of the foregoing endpoints. For example, the amino acid(s) can be present in the pharmaceutical formulation at a percentage concentration of about 0.1% to 10%, 0 about 0.2% to about 10%, about 0.5% to about 10%, about 0.1% to about 5%, about 0.1% to about 2%, about 0.2% to about 2%, about 0.5% to about 1%, and the like.

Any suitable polymers or polymeric compounds can be present in the pharmaceutical formulation. The polymer or polymeric compound can be, for example, a protein, a polysaccharide, a hydrogel, or any other suitable polymer or polymeric compound, many of which are known in the art. The polymers may preferably be polyanionic such as carboxymethylcellulose or poly(acrylic acid). For example, the polymer or polymeric compound can be recombinant human serum albumin (rHSA), serum albumin (SA), gelatin, hydroxyethyl starch (HES), chitosan, dextran (DEX70K, DEX40K), and polyvinylpyrrolidone (PVP40K).

The polymer(s) or polymeric compound(s) can be present in the pharmaceutical formulation in any suitable amount. The polymer(s) or polymeric compound(s) can be present in the pharmaceutical formulation at a percentage concentration (e.g., volume/volume percentage (% v/v); weight/volume percentage (% w/v); or weight/weight percentage (% w/w)). The polymer(s) or polymeric compound(s) can be present in the pharmaceutical formulation at a percentage concentration of about 0.1% or more, about 0.2% or more, about 0.3% or more, about 0.4% or more, about 0.5% or more, about 0.6% or more, about 0.7% or more, about 0.8% or more, about 0.9% or more, about 1% or more, about 2% or more, about 3% or more, about 4% or more, or about 5% or more. Alternatively, or in addition, the polymer(s) or polymeric compound(s) can be present in the pharmaceutical formulation at a percentage concentration of about 10% or less, about 9% or less, about 8% or less, about 7% or less, about 6% or less, about 5% or less, about 4% or less, about 3% or less, about 2% or less, or about 1% or less. The polymer(s) or polymeric compound(s) can be present in the pharmaceutical formulation at any percentage concentration within a range bounded by any of the foregoing endpoints. For example, the polymer(s) or polymeric compound(s) can be present in the pharmaceutical formulation at a percentage concentration of about 0.1% to about 10%, about 0.2% to 1 about 0%, about 0.5% to about 10%, about 0.1% to about 5%, about 0.1% to about 2%, about 0.2% to about 2%, about 0.5 to about 2%, about 0.1% to about 1%, about 0.2% to about 1%, about 0.5% to about 1%, and the like.

Any suitable chelating agent can be present in the pharmaceutical formulation. The chelating agent can be, for example, ethylenediaminetetraacetic acid (EDTA), an amidoxime compound (AOX), and/or dithiothreitol (DTT). The chelating agent can be present in the pharmaceutical formulation at any suitable concentration. The chelating agent can be present in the pharmaceutical formulation at a concentration of 10 μM or more, about 20 UM or more, about 30 μM or more, about 40 μM or more, about 50 μM or more, about 60 μM or more, about 70 μM or more, about 80 μM or more, about 90 μM or more, about 100 μM or more, about 120 μM or more, or about 150 μM or more. Alternatively, or in addition, the chelating agent can be present in the pharmaceutical formulation at a concentration of about 500 μM or less, about 400 μM or less, about 300 μM or less, about 200 μM or less, about 150 μM or less, about 140 μM or less, about 130 μM or less, about 120 μM or less, about 110 μM or less, about 100 μM or less, about 80 μM or less, about 70 μM or less, about 60 μM or less, or about 50 μM or less. The chelating agent can be present in the pharmaceutical formulation at any concentration within a range bounded by any of the foregoing endpoints. For example, the chelating agent can be present in the pharmaceutical formulation at a concentration of about 10 μM to about 500 μM, about 10 μM to about 200 μM, about 10 μM to about 150 μM, about 10 μM to about 100 μM, about 50 μM to about 500 μM, about 50 μM to about 200 μM, about 50 μM to about 150 μM, about 50 μM to about 100 μM, and the like.

Any suitable sugar can be present in the pharmaceutical formulation. The sugar can be, for example, one or more of sucrose, trehalose, mannose, and lactose. The sugar(s) can be present in the pharmaceutical formulation at any suitable concentration. The sugar(s) can be present in the pharmaceutical formulation at a concentration of about 0.1 mM or more, about 0.2 mM or more, about 0.4 mM or more, about 0.6 mM or more, about 0.8 mM or more, about 1 mM or more, about 1.2 mM or more about 1.4 mM or more about 1.6 mM or more about 1.8 mM or more, about 2 mM or more about 3 mM or more about 4 mM or more, about 5 mM or more, about 6 mM or more, about 7 mM or more, about 8 mM or more, about 9 mM or more, about 10 mM or more, about 20 mM or more, about 30 mM or more, about 40 mM or more, about 50 mM or more, about 60 mM or more, about 70 mM or more, about 80 mM or more, about 90 mM or more, or about 100 mM or more, about 200 mM or more, about 300 mM or more, about 400 mM or more, about 500 mM or more, about 600 mM or more, about 700 mM or more, about 800 mM or more, about 900 mM or more, about 1000 mM or more, or about 1500 mM or more. Alternatively, or in addition, the sugar(s) can be present in the pharmaceutical formulation at a concentration of about 2000 mM or less, about 1500 mM or less, about 1000 mM or less, about 900 mM or less, about 800 mM or less, about 700 mM or less, about 600 mM or less, about 500 mM or less, about 450 mM or less, about 400 mM or less, about 350 mM or less, about 300 mM or less, about 250 mM or less, about about 200 mM or less, about 150 mM or less, about 100 mM or less, about 50 mM or less, about 45 mM or less, about 40 mM or less, about 35 mM or less, about 30 mM or less, about 25 mM or less, about 20 mM or less, about 10 mM or less, about 9 mM or less, about 8 mM or less, about 7 mM or less, about 6 mM or less, about 5 mM or less, about 4 mM or less, about 3 mM or less, about 2 mM or less, about 1.8 mM or less, about 1.6 mM or less, about 1.4 mM or less, about 1.2 mM or less, about 1 mM or less, about 0.8 mM or less, about 0.6 mM or less, about 0.4 mM or less, or about 0.2 mM or less. The sugar(s) can be present in the pharmaceutical formulation at any concentration within a range bounded by any of the foregoing endpoints. For example, the sugar(s) can be present in the pharmaceutical formulation at a concentration of about 0.1 mM to about 2000 mM, about 0.1 mM to about 1500 mM, about 0.1 mM to about 1000 mM, about 0.1 mM to about 500 mM, about 0.1 mM to about 250 mM, about 0.1 mM to about 100 mM, about 0.1 to about 50 mM, about 0.1 mM to about 10 mM, about 1 mM to about 2000 mM, about 1 mM to about 1500 mM, about 1 mM to about 1000 mM, about 1 mM to about 500 mM, about 1 mM to about 250 mM, about 1 mM to about 100 mM, about 1 mM to about 50 mM, about 1 mM to about 10 mM, about 10 mM to about 2000 mM, about 10 mM to about 1500 mM, v10 mM to about 1000 mM, about 10 mM to about 500 mM, about 10 mM to about 250 mM, about 10 mM to about 100 mM, about 10 mM to about 50 mM, about 100 mM to about 2000 mM, about 100 mM to about 1500 mM, about 100 mM to about 1000 mM, about 100 mM to about 500 mM, about 100 mM to about 250 mM, about 500 mM to about 2000 mM, about 500 mM to about 1500 mM, about 500 mM to about 1000 mM, and the like.

In other embodiments, the sugar(s) is present in the pharmaceutical formulation at a percentage concentration (e.g., volume/volume percentage (% v/v); weight/volume percentage (% w/v); or weight/weight percentage (% w/w)). The sugar(s) can be present in the pharmaceutical formulation at a percentage concentration of about 0.1% or more, about 1% or more, about 5% or more, about 10% or more, about 15% or more, about 20% or more, about 30% or more, about 40% or more. Alternatively, or in addition, the sugar(s) can be present in the pharmaceutical formulation at a percentage concentration of about 50% or less, about 40% or less, about 30% or less, about 20% or less, about 15% or less, about 10% or less, about 5% or less, or about 1% or less. The sugar(s) can be present in the pharmaceutical formulation at any percentage concentration within a range bounded by any of the foregoing endpoints. For example, the sugar(s) can be present in the pharmaceutical formulation at a percentage concentration of about 0.1% to about 50%, about 1% to about 50%, about 10% to about 50%, about 0.1% to about 20%, about 1% to about 20%, about 10% to about 20%, about 0.1% to about 10%, about 1% to about 10%, and the like.

Any suitable polyol can be present in the pharmaceutical formulation. The polyol can be, for example, sorbitol and/or mannitol. The polyol can be present in the pharmaceutical formulation at any suitable concentration. The polyol can be present in the pharmaceutical formulation at a concentration of about 0.1 mM or more, about 1 mM or more, about 10 mM or more, about 20 mM or more, about 30 mM or more, about 40 mM or more, about 50 mM or more, about 60 mM or more, about 70 mM or more, about 80 mM or more, about 90 mM or more, about 100 mM or more, about 120 mM or more, about 140 mM or more, about 160 mM or more, about 180 mM or more, about 200 mM or more, about 250 mM or more, about 300 mM or more, about 350 mM or more, about 400 mM or more, about 450 mM or more, or about 500 mM or more. Alternatively, or in addition, the polyol can be present in the pharmaceutical formulation at a concentration of about 1000 mM or less, about 500 mM or less, about 450 mM or less, about 400 mM or less, about 350 mM or less, about 300 mM or less, about 250 mM or less, about 200 mM or less, about 180 mM or less, about 160 mM or less, about 140 mM or less, about 120 mM or less, about 100 mM or less, about 90 mM or less, about 80 mM or less, about 70 mM or less, about 60 mM or less, about 50 mM or less, about 40 mM or less, about 30 mM or less, about 20 mM or less, about 10 mM or less, or about 1 mM or less. The polyol can be present in the pharmaceutical formulation at any concentration within a range bounded by any of the foregoing endpoints. For example, the polyol can be present in the pharmaceutical formulation at a concentration of about 0.1 mM to about 1000 mM, about 0.1 mM to about 500 mM, about 0.1 mM to about 100 mM, about 1 mM to about 1000 mM, about 1 mM to about 500 mM, about 1 mM to about 100 mM, about 100 mM to about 1000 mM, about 100 mM to about 500 mM, and the like.

In other embodiments, the polyol is present in the pharmaceutical formulation at a percentage concentration (e.g., volume/volume percentage (% v/v); weight/volume percentage (% w/v); or weight/weight percentage (% w/w)). The polyol can be present in the pharmaceutical formulation at a percentage concentration of about 0.1% or more, about 1% or more, about 2% or more, about 3% or more, about 4% or more, or about 5% or more, about 10% or more, about 15% or more, about 20% or more, about 25% or more, about 30% or more, about 35% or more, about 40% or more, about 45% or more. Alternatively, or in addition, the polyol can be present in the pharmaceutical formulation at a percentage concentration of about 50% or less, about 45% or less, about 40% or less, about 35% or less, about 30% or less, about 25% or less, about 20% or less, about 15% or less, about 10% or less, about 5% or less, about 4% or less, about 3% or less, about 2% or less, or about 1% or less. The polyol can be present in the pharmaceutical formulation at any percentage concentration within a range bounded by any of the foregoing endpoints. For example, the polyol can be present in the pharmaceutical formulation at a percentage concentration of about 0.1% to about 50%, about 1% to about 50%, about 5% to about 50%, about 10% to about 50%, about 15% to about 50%, about 0.1% to about 25%, about 1% to about 25%, about 5% to about 25%, about 10% to about 25%, about 15% to about 25%, about 0.1% to about 15%, about 1% to about 15%, about 5% to about 15%, about 10% to about 15%, about 0.1% to about 10%, about 1% to about 10%, about 5% to about 10%, about 0.1% to about 5%, about 1% to about 5%, and the like.

In one embodiment, the pharmaceutical formulation comprises the inventive influenza virus, about 0.5 M sucrose, about 0.1 M or about 0.5 M mannose, about 0.3 M or about 0.5 M trehalose, about 50% SPG, and about 0.05% polysorbate 20. In another embodiment, the pharmaceutical formulation comprises the inventive influenza virus, about 0.5 M sucrose, about 0.3 M trehalose, and about 0.05% polysorbate 20.

The at least one pharmaceutically acceptable carrier or excipient can be a component that serves to bind the ingredients of the pharmaceutical formulation (e.g., a binder). The binder may include, but is not limited to, proteins (e.g., gelatin), polymers (e.g., polyethylene glycol, polyvinylpyrrolidone), and/or polysaccharides or derivatives thereof (e.g., starch and cellulose). The at least one pharmaceutically acceptable carrier or excipient can be a component that increases bulk of the pharmaceutical formulation (e.g., a bulking agent, diluent, and/or filler). Such bulking agents may include, but are not limited to, polysaccharides or derivatives thereof, sugars, and/or inorganic compounds. The pharmaceutically acceptable carrier or excipient can be a component that enhances taste and/or appearance of the pharmaceutical formulation (e.g., a flavor, sweetener, and/or color). The pharmaceutically acceptable carrier or excipient can be a component that moisture-proofs the pharmaceutical formulation by absorbing or adsorbing liquids or gases (e.g., a sorbent). A sorbent includes, but is not limited to, starch, calcium phosphate, and/or colloidal silicon dioxide. The pharmaceutically acceptable carrier or excipient can be a component that promotes dissolution of the pharmaceutical formulation (e.g., a disintegrant), such as a starch, cellulose and/or any other polymer known in the art, or derivative thereof (e.g., cross-linked polyvinylpyrrolidone or sodium carboxymethylcellulose).

In some embodiments, the pharmaceutically acceptable carrier or excipient is a component that reduces interparticle adhesion and/or optimizes product flow in and during manufacture of a pharmaceutical formulation (e.g., a glidant). Examples of glidants include, but are not limited to, talc, colloidal silicon dioxide, and corn starch. The pharmaceutically acceptable carrier or excipient can be a component that provides non-sticking properties, such as reducing adhesion between the ingredients and, for example, the punch faces or lubricant in and during manufacture of a pharmaceutical formulation (e.g., an anti-adherent), particularly when the pharmaceutical formulation is formulated as an oral preparation. For example, the anti-adherent may comprise magnesium stearate. In other embodiments, the pharmaceutically acceptable carrier or excipient can be a component that reduces clumping of ingredients and/or reduce friction between, for example, the surface of a pharmaceutical formulation, i.e., formulated as an oral preparation, and the die wall during manufacture (e.g., a lubricant). Both water-soluble or water-insoluble lubricants may be used according to certain embodiments, such as magnesium stearate, stearic acid, vegetable oil, mineral oil, polyethylene glycol, and/or sodium lauryl sulfate. The pharmaceutically acceptable carrier or excipient can be a component that acts as a coating agent. Coating agents include, but are not limited to, gelatin and/or cellulose-based coating agents (e.g., hydroxypropyl methylcellulose).

Other suitable binders, flavors, sweeteners, colors, disintegrants, glidants, anti-adherents, lubricants, and coating agents are well known and readily identifiable in the art.

The pharmaceutical formulation can further comprise a therapeutic agent (e.g., a chemotherapeutic or anti-inflammatory agent). The pharmaceutical formulation can also comprise an agent that triggers an immune response separate from the influenza virus. Such additional components other than the inventive influenza virus can be present in any suitable amount(s).

The additional components can be mixed with the other components to form the pharmaceutical formulation prior to presentation to the immune system. The additional components can also be presented to the immune system separately from the pharmaceutical formulation. For example, the additional components and the pharmaceutical formulation can be presented to the immune system (e.g., administered to an organism) separately. When the additional components and the pharmaceutical formulation are administered separately, the additional components and the pharmaceutical formulation can be administered to the same site of the organism being immunized.

In one embodiment of the pharmaceutical formulation, the pharmaceutical formulation is a virus vaccine. The virus vaccine may be a live, attenuated virus vaccine or an inactivated virus vaccine (e.g., a whole virus vaccine, split virus vaccine, or subunit vaccine). The virus vaccine may be formulated with multiple influenza viral backbone subtypes (i.e., with different hemagglutinin and neuraminidase subtypes for influenza A and either Yamagata or Victoria lineages for influenza B) as a monovalent vaccine, a bivalent vaccine (e.g., H1H3, H1By H1Bv, H3Bv, or BvBy), a trivalent vaccine (e.g., H1H3By, H1H3Bv, BvByH1, or BvByH3), or a quadrivalent vaccine (e.g., H1H3ByBv). For example, the vaccine may comprise multiple embodiments of the inventive recombinant virus. In some embodiments, the vaccine may further comprise at least one recombinant virus different from the recombinant virus of the invention.

The virus vaccine can be formulated into a composition for any suitable means of administration. For example, the virus vaccine can be formulated as an oral preparation (e.g., capsule, tablet, or oral film), a spray (e.g., nasal spray), or any composition suitable for intranasal administration, or parenteral administration, e.g., intravenous, intramuscular, intradermal or subcutaneous administration, such as an aqueous or non-aqueous emulsion, solution, or suspension.

### EMBODIMENTS

(1) A recombinant virus comprising an influenza viral backbone, wherein the influenza viral backbone comprises PB1, PB2, PA, NP, M, NS, HA, and NA gene segments, wherein at least one of the PB1, PB2, PA, NP, M, NS, HA, and NA gene segments comprises at least one nucleotide sequence that encodes one or more antigens, and wherein (a) the PB1 gene segment encodes a PB1 protein having an amino acid sequence comprising selected amino acids, wherein the selected amino acids comprise a leucine at position 40 and a tryptophan at position 180, and at least one of an asparagine at position 464, an isoleucine at position 563, or a serine at position 607, and wherein the PB1 gene segment optionally comprises a cytosine to uracil promoter mutation at nucleotide position 4; (b) the PB2 gene segment encodes a PB2 protein having an amino acid sequence comprising selected amino acids, wherein the selected amino acids comprise a valine at position 504, and optionally an isoleucine at position 467 and a valine at position 529, and wherein the PB2 gene segment optionally comprises a cytosine to uracil promoter mutation at nucleotide position 4; (c) the PA gene segment encodes a PA protein having an amino acid sequence comprising selected amino acids, wherein the selected amino acids comprise a lysine at position 401, and wherein the PA gene segment optionally comprises a cytosine to uracil promoter mutation at nucleotide position 4; (d) the NP gene segment encodes an NP protein having an amino acid sequence comprising selected amino acids, wherein the selected amino acids comprise a leucine at position 116, and at least one of a lysine at position 294 or an arginine at position 311; and (e) the NS gene segment encodes an NS1 protein having amino acid sequence comprising selected amino acids, wherein the selected amino acids comprise a proline at position 30, a lysine at position 55, and a lysine at position 118.
(2) The recombinant virus of embodiment 1, wherein the antigen is an immunogenic fragment of SARS-COV-2 spike glycoprotein.
(3) The recombinant virus of embodiment 1 or 2, wherein the M gene segment comprises at least one nucleotide sequence that encodes an antigen, wherein the antigen is an immunogenic fragment of SARS-COV-2 spike glycoprotein.
(4) The recombinant virus of any of embodiments 1-3, wherein the M gene segment
encodes a mutated M2 protein.
(5) The recombinant virus of embodiment 4, wherein the M gene segment encodes a protein comprising at least one linker protein and FLAG epitope tag.
(6) The recombinant virus of any one of embodiments 1-5, wherein the M segment encodes a protein comprising any one of SEQ ID NOs: 1-14 and 92-96.
(7) A recombinant virus comprising an influenza viral backbone, wherein the influenza viral backbone comprises PB1, PB2, PA, NP, M, NS, HA, and NA gene segments comprises at least one nucleotide sequence that encodes one or more antigens, wherein (a) the PA gene segment comprises a thymine at nucleotide position 2272; (b) the NP gene segment encodes a NP protein having an amino acid sequence comprising selected amino acids, wherein the selected amino acids comprise a serine at position 40, an asparagine or glycine at position 161, a threonine at position 204, and optionally a valine at position 93; and (c) the NS gene segment comprises a guanine at nucleotide position 39, and wherein the NS gene segment encodes an NS protein having an amino acid sequence comprising selected amino acids, wherein the selected amino acids comprise a glutamine at position 176.
(8) The recombinant virus of embodiment 7, wherein the antigen is an immunogenic fragment of SARS-COV-2 spike glycoprotein.
(9) The recombinant virus of embodiment 7 or 8, wherein the M gene segment comprises at least one nucleotide sequence that encodes an antigen, wherein the antigen is an immunogenic fragment of SARS-COV-2 spike glycoprotein and further encodes a mutated BM2 protein.
(10) The recombinant virus of any one of embodiments 1-9, wherein the NS gene segment comprises at least one nucleotide sequence that encodes one or more antigens.
(11) The recombinant virus of any one of embodiments 1-10 wherein the antigen is an immunogenic fragment of SARS-COV-2 spike glycoprotein.
(12) (The recombinant virus of any one of embodiments 1-11, wherein the NS gene segment encodes a (1) a NS1 protein, (2) at least one flexible linker protein, (3) an immunogenic fragment of SARS-COV-2 spike glycoprotein, (4) at least one cleavable cleavage sequence, and (5) a NEP protein.

The recombinant virus of embodiment 12, wherein the at least one cleavable cleavage sequence is a T2A peptide sequence or a P2A peptide sequence.
(14) The recombinant virus of any one of embodiments 1-13, wherein the NS gene segment encodes a protein comprising any one of SEQ ID NOs: 97-104.
(15) The recombinant virus of embodiment 1 or 7, wherein each of the M and NS gene segments comprises at least one nucleotide sequence that encodes one or more antigens, and wherein the antigens are immunogenic fragments of SARS-COV-2 spike glycoprotein.
(16) The recombinant virus of embodiment 1 or 7, wherein each of the NA and NS gene segments comprises at least one nucleotide sequence that encodes one or more antigens, and wherein the antigens are immunogenic fragments of SARS-COV-2 glycoprotein.
(17) The recombinant virus of embodiment 1 or 7, wherein each of the M and NA gene segments comprises at least one nucleotide sequence that encodes one or more antigens, and wherein the antigens are immunogenic fragments of SARS-COV-2 glycoprotein.
(18) The recombinant virus of embodiment 1 or 7, wherein each of the M and HA gene segments comprises at least one nucleotide sequence that encodes one or more antigens, and wherein the antigens are immunogenic fragments of SARS-COV-2 glycoprotein.
(19) The recombinant virus of embodiment 1 or 7, wherein each of the NS and NA gene segments comprises at least one nucleotide sequence that encodes one or more antigens, and wherein the antigens are immunogenic fragments of SARS-COV-2 glycoprotein.
(20) The recombinant virus of embodiment 1 or 7, wherein each of the NS and HA gene segments comprises at least one nucleotide sequence that encodes one or more antigens, and wherein the antigens are immunogenic fragments of SARS-COV-2 spike glycoprotein.
(21) The recombinant virus of any one of embodiments 1-20, wherein the virus is capable of replication in human cells.
(22) The recombinant virus of any one of embodiments 1-21, wherein the virus has enhanced growth as compared to a recombinant virus that is the same except without the selected amino acids in Vero cells under the same conditions.
(23) The recombinant virus of any one of embodiments 1-22, wherein the gene segment that comprises at least one nucleotide sequence that encodes one or more antigens further comprises a downstream duplication and wherein the downstream duplication comprises at least one silent nucleotide mutation.
(24) A pharmaceutical formulation comprising the recombinant virus of any one of embodiments 1-23.
(25) The pharmaceutical formulation of embodiment 24, wherein the vaccine is formulated as a monovalent vaccine.
(26) The pharmaceutical formulation of embodiment 24, wherein the vaccine is formulated as a bivalent vaccine.
(27) The pharmaceutical formulation of embodiment 24, wherein the vaccine is formulated as a trivalent vaccine.
(28) The pharmaceutical formulation of embodiment 24, wherein the vaccine is formulated as a quadrivalent vaccine.
(29) A method of eliciting an immune response in a mammal, the method comprising administering the recombinant virus of any one of embodiments 1-23 or the pharmaceutical formulation of any one of embodiments 24-28 to the mammal, thereby eliciting an immune response to the antigen in the mammal.
(30) The method of embodiment 29, wherein the mammal is a human

### EXAMPLES

The following examples further illustrate the invention but, of course, should not be construed as in any way limiting its scope.

### Example 1

This example demonstrates the methods used to select the MHC I peptides used in the influenza vectors. The peptides were suitable for insertion into the M2, BM2, and NS genes.

Peptide antigens for vaccine were selected based on their ability to stimulate immune response from the broadest possible number of MHC genotypes, thus providing benefit to the largest possible number of potential vaccines. This approach was taken because high specificity to the interaction of MHC class I molecules on the cell surface and cognate antigen peptides bound and displayed for presentation to immune effector T-cells. Antigen peptides with higher specific MHC I affinity elicit a stronger immune response upon vaccination. Many models have been developed that were able to predict the affinity of any peptide sequence for a given MHC Class I molecule. This interaction is also allele-specific amongst the many known MHC Class I genotypes found worldwide within individuals from differing genetic backgrounds. Thus, any single peptide will have a different affinity for a MHC Class I receptor that is dependent upon the individual MHC I genotype.

The S1 or spike surface glycoprotein from the SARS-COV-2 coronavirus was used as the target antigen protein for identification of the best peptide. The amino acid sequence of S1 protein (SEQ ID NO: 77) was predicted from the complete genome sequence of Severe acute respiratory syndrome coronavirus 2 isolate Wuhan-Hu-1 (Genbank NC_045512.2) by standard codon usage table. The primary S1 protein sequence was used to identify the best MHC Class I compatible 9-mer peptides by prediction of peptide affinities across a 27-member human MHC Class I allele panel. Peptide predictions were ranked by predicted consensus percentile rank across all predictors in an ensemble; those with percentile rank≤1 were selected. Cluster analysis was applied to these peptides using known methods (see e.g., Dhanda et al., Front. Immunol., 9:1369 (2018)), picking epitope clusters predicted to have high affinity for many MHC Class I molecules from high genetic diversity. Top scoring peptides were ranked using a two-step process. Initially peptides were ranked by cluster connectivity to locate peptide affinity “smears” that were regions of high predicted affinity where multiple 9-mers were tiled (i.e., peptides align and overlap). In this way peptide smears that were longer than 9 residues can be identified and targeted for inclusion within the vaccine. The number of times a given peptide was scored in the top 1% was tabulated to score the cumulative hit count and median epitope rank to select the top peptide smears that were expected to bind MHC I within human subjects with high genetic diversity. This method is described in more detail below.

Predicting Epitopes with MHC-I Activity

Immune Epitope Database and Analysis Resource's (IEDB) TepiTool was used to extract MHC I-relevant epitope predictions from the amino acid sequence for SARS-COV-2 spike protein. Epitopes were predicted for human MHC-I alleles, using a 27-allele panel; epitope sizes were allowed to range from 8-mers to 11-mers. Duplicate peptides were removed, and the IEDB recommended prediction method was used. Peptides were selected with predicted consensus percentile rank less than or equal to 1, producing 647 epitopes meeting the rank condition and 136566 overall tuples (epitope, allele, predictor, rank).

### Clustering Epitopes to Smears

IEDB's Epitope Cluster Analysis tool was used to cluster the previously selected epitopes into a cluster of relevant epitopes, herein referred to as an “epitope smear.” A minimum sequence identity threshold of 70% was chosen, with no size filters placed upon the epitope list. Predicted epitopes were clustered into smears as those with consensus percentile rank less than or equal to 1. The cluster-breaking clustering algorithm was used, and clusters with their epitope alignments were output into a comma separated values (CSV) file format. In this use case, the clusters were effectively ordered by decreasing connectedness. Using this method, clusters were ranked in lexicographic cluster-subcluster sorted order.

### Analyzing Smears

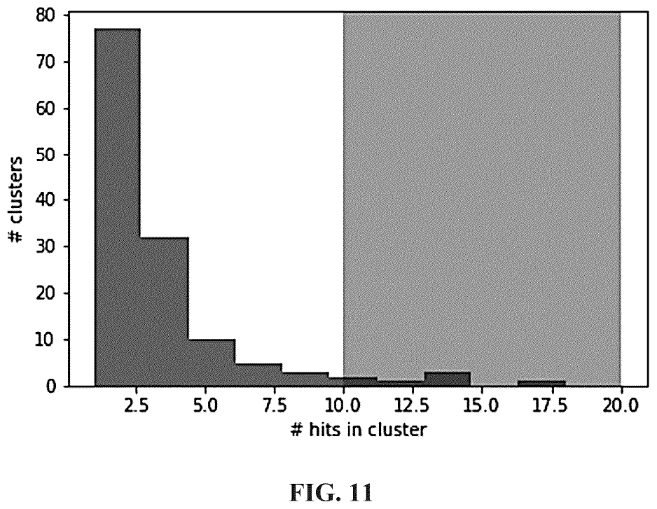

Using a Python script, clusters were ingested, normalized, and then aligned to the spike S1 protein open reading frame obtained from GenBank RefSeq genome NC 045512.2. Various qualitative statistical analyses were performed on the distributions of hits, sequence length, etc., to inform smear selection. Hit count (number of epitopes per cluster) and median rank (median of consensus percentile ranks for each epitope in the cluster) were added as columns. The total number of hits was a useful metric to select top candidate smears sequences. Only a small subset of peptide smears were found to contain greater than 10 hits per cluster (FIG. 11). By setting an arbitrary cut-off of 9 hits per cluster only 8 total smears were identified within the SARS-COV-2 S1 protein of 1273 amino acids in length.

The results from statistical analyses were compiled and re-output to CSV format for visualization. Top candidate smears were compared to each other and curated manually. In three cases candidate smears were overlapping or nearly adjacent which that allowed combination into super consensus smear sequences that span two candidate sequences. The selected 8 candidate smears are shown in Table 1.

TABLE 1
Top 8 Candidate MHC I Compatible Peptide Smears SARS-CoV-2 S1 Protein

| Smear | | | | | | | |
|---|---|---|---|---|---|---|---|
| Cluster | | | | | | | |
| Sub- | Peptide | | | | | | |
| Cluster | Type | Peptide Sequence | Description | Start | End | Length | #Hits |
| 1.1 | Consensus | QELGKYEQYIKWPWYIWLGFIAGLI (SEQ ID | Spike HR2 + | 1201 | 1225 | 25 | 18 |
| | | NO: 21) | Transmembrane | | | | |
| 2.1 | Consensus | NLDSKVGGNYNYLYRLFRK (SEQ ID NO: 22) | | 440 | 458 | 19 | 12 |
| 2.2 | Consensus | YRLFRKSNLKPFER (SEQ ID NO: 23) | Spike RBD | 453 | 466 | 14 | 6 |
| 2.X | Super | NLDSKVGGNYNYLYRLFRKSNLKPFER (SEQ ID | | 440 | 466 | 27 | 18 |
| | Consensus | NO: 24) | | | | | |
| 3.1 | Consensus | AALQIPFAMQMAYRENGIGV (SEQ ID NO: 25) | Spike Other | 892 | 911 | 20 | 14 |
| 4.1 | Consensus | EVFNATRFASVYAWNRKRI (SEQ ID NO: 26) | | 340 | 358 | 19 | 13 |
| 4.2 | Singleton | GEVFNATRF (SEQ ID NO: 27) | Spike RBD | 339 | 347 | 9 | 1 |
| 4.X | Super | GEVFNATRFASVYAWNRKRI (SEQ ID NO: 28) | | 339 | 358 | 20 | 14 |
| | Consensus | | | | | | |
| 5.1 | Consensus | YSVLYNSASFSTFKCYGV (SEQ ID NO: 29) | Spike RBD | 365 | 382 | 18 | 13 |
| 6.1 | Consensus | SPRRARSVASQSIIAY (SEQ ID NO: 30) | Spike Cleavage | 680 | 695 | 16 | 11 |
| | | | Site | | | | |
| 7.1 | Consensus | MSLGAENSVAYSNNSIAI (SEQ ID NO: 31) | Spike Other | 697 | 714 | 18 | 10 |
| 6 + 7 | Super | SPRRARSVASQSIIAYTMSLGAENSVAYSNNSIAI | Spike Cleavage | 680 | 714 | 35 | 21 |
| | Consensus | (SEQ ID NO: 32) | Site | | | | |
| 8.1 | Consensus | LPPLLTDEMIAQYTSAL (SEQ ID NO: 33) | Spike Other | 861 | 877 | 17 | 7 |
| 8.2 | Consensus | MIAQYTSALL (SEQ ID NO: 34) | | 869 | 878 | 10 | 2 |
| 8.X | Super | LPPLLTDEMIAQYTSALL (SEQ ID NO: 35) | | 861 | 878 | 18 | 9 |
| | Consensus | | | | | | |

### Optimal Spacer Design

Epitopes were assembled into concatemers by extension from a published algorithm (Schubert et al., Genome Medicine, 8(1): 9 (2016)) previously implemented in Fred2 (Schubert et al., Bioinformatics, 32(13): 1367-4803, 2044 (2016)). Spacers and epitope ordering should both be optimized to minimize neoepitope formation and to maximize MHC processing cut probability in the spacer regions. In theory, manipulating carefully chosen entries in the cost matrix to plus or minus infinity could provide optimized spacers, but the ILP solver in use (CBC) fails to solve the problem with infinite coefficients in the objective. In the new method the k-mer spacers were optimized for kmax={3, 6}. To properly optimize cutting and neoepitope formation at the termini of epitope chain inserts that were bounded at the C or N terminus by existing amino acid strings, a four-part modification was designed to improve upon the previous methods:
1. C/N-terminal bounding strings were added to the peptide set in the Traveling Salesman model (formulated as an ILP in Miller-Tucker-Zemlin form),
2. Spacer optimization was modified to generate spacers to the bounding sequences, on the correct terminus for all epitope peptides,
3. Constraints were added to the model to enforce correct ordering of epitopes and bounding sequences,
4. The objective was modified to ignore the missing entries in a TSP cost matrix introduced by the bounding sequences.

TABLE 2
M2 and BM2 Protein SARS-CoV2 Protein Amino Acid Sequences: Influenza A/PR/8
M2, Influenza B/Lee/40 BM2 and Chimeric Fusion Proteins Thereof

A/PR/8 M2 (SEQ ID NO: 1) 97 amino acid wild-type influenza A/Puerto Rico/1934 (H1N1)
M2 protein.
MSLLTEVETPIRNEWGCRCNGSSDPLTIAANIIGILHLTLWILDRLFFKCIYRRFKYGLK
GGPSTEGVPKSMREEYRKEQQSAVDADDGHFVSIELE
UPPER CASE = M2 Protein
UNDER LINE = Transmembrane Domain
ITALIC = Canonical Proton Channel Residues
B/Lee/40 BM2 (SEQ ID NO: 2) 109 amino acid wild-type influenza B/Lee/1940 BM2 protein.
MLEPLQILSICSFILSALHFMAWTIGHLNQIRRGVNLKIQIRNPNKEAINREVSILR
HNYQKEIQAKETMKKILSDNMEVLGDHIVVEGLSTDEIIKMGETVLEVEELQ
BOLD UPPER CASE = BM2 Protein
BOLD UNDER LINE = BM2 Transmembrane Domain
ITALIC = Canonical Proton Channel Residues
A/PR/8 M2 (SEQ ID NO: 3) 25 amino acid influenza A/Puerto Rico/1934 (H1N1) M2
truncation protein.
MSLLTEVETPIRNEWGCRCNGSSDP
UPPER CASE = M2 Protein
/PR/8 M2 (SEQ ID NO: 4) 33 amino acid influenza A/Puerto Rico/1934 (H1N1) M2
truncation FLAG epitope tag fusion protein.
MSLLTEVETPIRNEWGCRCNGSSDPAGAGDYKDDDDK
UNDERLINE UPPER CASE = M2 Protein amino acids 1 to 25
ITALIC UPPER CASE = Linker between antigen and epitope tag
BOLD UPPER CASE = FLAG epitope tag
A/PR/8 M2SR M2e + P delTM TM1.1 (SEQ ID NO: 5) 56 amino acid influenza A/Puerto
Rico/1934 (H1N1) M2 truncation, SARS-CoV-2 S1 antigen fusion protein.
MSLLTEVETPIRNEWGCRCNGSSDPIDLQELGKYEQYIKWPWYIWLGFIAGLIAIV
UNDERLINE UPPER CASE = M2 Protein amino acids 1 to 25
UPPER CASE = 31 amino acid selected MHC epitope smear, TM1.1, SARS-CoV-2 S1
protein amino acids 1198 to 1228
A/PR/8 M2SR M2e + P delTM TM1.1 FLAG (SEQ ID NO: 6) 68 amino acid influenza
A/Puerto Rico/1934 (H1N1) M2 truncation, SARS-CoV-2 S1 antigen, FLAG epitope tag
fusion protein.
MSLLTEVETPIRNEWGCRCNGSSDPIDLQELGKYEQYIKWPWYIWLGFIAGLIAIVAGA
GDYKDDDDK
UNDERLINE UPPER CASE = M2 Protein amino acids 1 to 25
UPPER CASE = 31 amino acid selected MHC epitope smear, TM1.1, SARS-CoV-2 S1
protein amino acids 1198 to 1228
ITALIC UPPER CASE = Linker between antigen and epitope tag
BOLD UPPER CASE = FLAG epitope tag
A/PR/8 M2SR M2e + P delTM RBD 2.X (SEQ ID NO: 7) 52 amino acid influenza A/Puerto
Rico/1934 (H1N1) M2 truncation, SARS-CoV-2 S1 antigen fusion protein.
MSLLTEVETPIRNEWGCRCNGSSDPNLDSKVGGNYNYLYRLFRKSNLKPFER
UNDERLINE UPPER CASE = M2 Protein amino acids 1 to 25
UPPER CASE = 27 amino acid selected MHC epitope, RBD2.X, SARS-CoV-2 S1 protein
amino acids 440 to 466
A/PR/8 M2SR M2e + P delTM RBD 2.X FLAG (SEQ ID NO: 8) 64 amino acid influenza
A/Puerto Rico/1934 (H1N1) M2 truncation, SARS-CoV-2 S1 antigen, FLAG epitope tag
fusion protein.
MSLLTEVETPIRNEWGCRCNGSSDPNLDSKVGGNYNYLYRLFRKSNLKPFERAGAGD
YKDDDDK
UNDERLINE UPPER CASE = M2 Protein amino acids 1 to 25
UPPER CASE = 27 amino acid selected MHC epitope smear, RBD 2.X, SARS-CoV-2 S1
protein amino acids 440 to 466
ITALIC UPPER CASE = Linker between antigen and epitope tag
BOLD UPPER CASE = FLAG epitope tag
A/PR/8 M2SR M2e + P delTM RBD 5.1 (SEQ ID NO: 9) 43 amino acid influenza A/Puerto
Rico/1934 (H1N1) M2 truncation, SARS-CoV-2 S1 antigen fusion protein.
MSLLTEVETPIRNEWGCRCNGSSDPYSVLYNSASFSTFKCYGV
UNDERLINE UPPER CASE = M2 Protein amino acids 1 to 25
UPPER CASE = 18 amino acid selected MHC epitope smear, RBD2.X, SARS-CoV-2 S1
protein amino acids 365 to 382
A/PR/8 M2SR M2e + P delTM RBD 5.1 FLAG (SEQ ID NO: 10) 55 amino acid influenza
A/Puerto Rico/1934 (H1N1) M2 truncation, SARS-CoV-2 S1 antigen, FLAG epitope tag
fusion protein.
MSLLTEVETPIRNEWGCRCNGSSDPYSVLYNSASFSTFKCYGVAGAGDYKDDDDK
UNDERLINE UPPER CASE = M2 Protein amino acids 1 to 25
UPPER CASE = 18 amino acid selected MHC epitope smear, RBD 5.1, SARS-CoV-2 S1
protein amino acids 365 to 382
ITALIC UPPER CASE = Linker between antigen and epitope tag
BOLD UPPER CASE = FLAG epitope tag
A/PR/8 M2SR M2e + P delTM Concatemer 3MAX (SEQ ID NO: 11) 205 amino acid influenza
A/Puerto Rico/1934 (H1N1) M2 truncation, SARS-CoV-2 S1 antigen smear concatemer
fusion protein.
MSLLTEVETPIRNEWGCRCNGSSDPLPPLLTDEMIAQYTSALLMNDNLDSKVGGNYN
YLYRLFRKSNLKPFERWNWSPRRARSVASQSIIAYSNWAALQIPFAMQMAYRFNGIG
VSWYSVLYNSASFSTFKCYGVSWGEVFNATRFASVYAWNRKRIGWMSLGAENSVA
YSNNSIAIYFWQELGKYEQYIKWPWYIWLGFIAGLI
UNDERLINE UPPER CASE = M2 Protein amino acids 1 to 25
UPPER CASE = 8 selected MHC epitope smears
UNDERLINE BOLD UPPER CASE = Linker of maximum length 3 amino acids between
peptide smears
A/PR/8 M2SR M2e + P delTM Concatemer 3MAX FLAG (SEQ ID NO: 12) 217 amino acid
influenza A/Puerto Rico/1934 (H1N1) M2 truncation, SARS-CoV-2 S1 antigen smear
concatemer fusion protein.
MSLLTEVETPIRNEWGCRCNGSSDPLPPLLTDEMIAQYTSALLMNDNLDSKVGGNYN
YLYRLFRKSNLKPFERWNWSPRRARSVASQSIIAYSNWAALQIPFAMQMAYRFNGIG
VSWYSVLYNSASFSTFKCYGVSWGEVFNATRFASVYAWNRKRIGWMSLGAENSVA
YSNNSIAIYFWQELGKYEQYIKWPWYIWLGFIAGLIAGAGDYKDDDDK
UNDERLINE UPPER CASE = M2 Protein amino acids 1 to 25
UPPER CASE = 8 selected MHC epitope smears, SARS-CoV-2 S1 protein
UNDERLINE BOLD UPPER CASE = Linker of maximum length 3 amino acids between
peptide smears
ITALIC UPPER CASE = Linker between antigen and epitope tag
BOLD UPPER CASE = FLAG epitope tag
A/PR/8 M2SR M2e + P delTM Concatemer 6MAX (SEQ ID NO: 13) 205 amino acid influenza
A/Puerto Rico/1934 (H1N1) M2 truncation, SARS-CoV-2 S1 antigen smear concatemer
fusion protein.
MSLLTEVETPIRNEWGCRCNGSSDPLPPLLTDEMIAQYTSALLMNDNLDSKVGGNYN
YLYRLFRKSNLKPFERWNWSPRRARSVASQSIIAYSNWAALQIPFAMQMAYRFNGIG
VSWYSVLYNSASFSTFKCYGVSWGEVFNATRFASVYAWNRKRIGWMSLGAENSVA
YSNNSIAIYFWQELGKYEQYIKWPWYIWLGFIAGLI
UNDERLINE UPPER CASE = M2 Protein amino acids 1 to 25
UPPER CASE = 8 selected MHC epitope smears
UNDERLINE BOLD UPPER CASE = Linker of maximum length 6 amino acids between
peptide smears
A/PR/8 M2SR M2e + P delTM Concatemer 6MAX FLAG (SEQ ID NO: 14) 217 amino acid
influenza A/Puerto Rico/1934 (H1N1) M2 truncation, SARS-CoV-2 S1 antigen smear
concatemer fusion protein.
MSLLTEVETPIRNEWGCRCNGSSDPLPPLLTDEMIAQYTSALLMNDNLDSKVGGNYN
YLYRLFRKSNLKPFERWNWSPRRARSVASQSIIAYSNWAALQIPFAMQMAYRFNGIG
VSWYSVLYNSASFSTFKCYGVSWGEVFNATRFASVYAWNRKRIGWMSLGAENSVA
YSNNSIAIYFWQELGKYEQYIKWPWYIWLGFIAGLIAGAGDYKDDDDK
UNDERLINE UPPER CASE = M2 Protein amino acids 1 to 25
UPPER CASE = 8 selected MHC epitope smears, SARS-CoV-2 S1 protein
UNDERLINE BOLD UPPER CASE = Linker of maximum length 6 amino acids between
peptide smears
ITALIC UPPER CASE = Linker between antigen and epitope tag
BOLD UPPER CASE = FLAG epitope tag

TABLE 3
cDNA Sequences Encoding M2 and Chimeric M2
Protein Amino Acid Sequences: Influenza
A/PR/8 M2, Influenza B/Lee/40 BM2, Codon-
Optimized Influenza A/PR/8 M2, Codon-
Optimized Influenza B/Lee/40 BM2, and Codon-
Optimized Chimeric Fusion Proteins Thereof.

A/PR/8 M2 (SEQ ID NO: 15) wild-type influenza
A/Puerto Rico/1934 (H1N1) M2 cDNA.
5′ATGAGTCTTCTAACCGAGGTCGAAACGCCTATCAGAAACGAATGGGGG
TGCAGATGCAACGGTTCAAGTGATCCTCTCACTATTGCCGCAAATATCAT
TGGGATCTTGCACTTGACATTGTGGATTCTTGATCGTCTTTTTTTCAAAT
GCATTTACCGTCGCTTTAAATACGGACTGAAAGGAGGGCCTTCTACGGAA
GGAGTGCCAAAGTCTATGAGGGAAGAATATCGAAAGGAACAGCAGAGTGC
TGTGGATGCTGACGATGGTCATTTTGTCAGCATAGAGCTGGAGtaa
UNDER LINE = Start Codon
Small Case = Stop Codon
B/Florida/06/2006 BM2 (SEQ ID NO: 16) wild-
type influenza B/Florida/06/2006 BM2 cDNA.
5′ATGCTCGAACCACTTCAGATTCTTTCAATTTGTTCTTTCATTTTATCA
GCTCTCCATTTCATGGCTTGGACAATAGGGCATTTGAATCAAATAAGAAG
AGGGGTAAACCTGAAAATACAAATAAGGAATCCAAATAAGGAGGCAATAA
ACAGAGAGGTGTCAATTCTGAGACACAATTACCAAAAGGAAATCCAAGCC
AAAGAAACAATGAAGAAAATACTCTCTGACAACATGGAAGTATTGGGTGA
CCACATAGTAGTTGAAGGGCTTTCAACTGATGAGATAATAAAAATGGGTG
AAACAGTTTTGGAGGTGGAAGAATTGCAAtaa
BOLD UNDER LINE = BM2 Start Codon
Bold Small Case = BM2 Stop Codon
A M2 OPT (SEQ ID NO: 17) codon-optimized
wild-type influenza A/Puerto Rico/1934
(H1N1) M2 cDNA.
5′ATGTCCCTGCTGACCGAAGTGGAAACTCCTATTAGAAACGAGTGGGGC
TGTAGATGTAACGGCTCAAGCGACCCTCTGACCATTGCTGCCAACATCAT
TGGCATCCTGCACCTGACCCTGTGGATTCTGGACCGACTGTTCTTTAAGT
GCATCTACCGGAGATTCAAGTATGGACTGAAAGGAGGACCAAGCACAGAG
GGAGTGCCTAAATCCATGAGGGAGGAATACCGCAAAGAGCAGCAGAGCGC
CGTGGACGCAGATGATGGACATTTCGTGAGCATTGAACTGGAAtga
UNDER LINE = Start Codon
Small Case = Stop Codon
BM2 OPT (SEQ ID NO: 18) codon-optimized
wild-type influenza B/Lee/1940 BM2 cDNA.
5′ATGCTGGAACCACTGCAGATCCTGAGTATTTGCTCTTTTATCCTGAGC
GCACTGCACTTTATGGCCTGGACTATCGGGCACCTGAACCAGATCAGAAG
GGGCGTGAACCTGAAGATCCAGATCAGAAACCCAAACAAGGAGGCCATCA
ACCGCGAAGTGAGCATCCTGAGACACAATTACCAGAAGGAGATCCAGGCT
AAAGAAACCATGAAGAAAATCCTGTCTGACAATATGGAGGTGCTGGGCGA
TCACATCGTGGTGGAAGGACTGAGCACCGACGAAATCATCAAAATGGGCG
AGACTGTCCTGGAAGTGGAAGAACTGCAGtaa
BOLD UNDER LINE = BM2 Start Codon
Bold Small Case = BM2 Stop Codon

### Example 2

This example demonstrates successful expression of a SARS-COV-2 receptor binding domain (RBD) antigen from an influenza A M2SR vector.

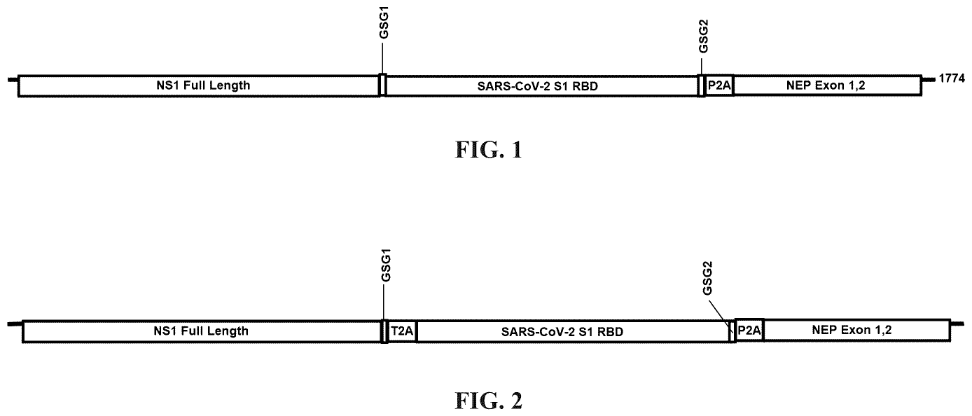

To express the SARS-COV-2 RBD antigen from the influenza A M2SR vector an engineered NS segment 8 was constructed synthetically (FIG. 1). The designed gene was then inserted into a RNA Pol I vector for expression as negative sense vRNA. The segment 8 was designed to express a single fusion polypeptide of three major open reading frames (ORFs): first the complete influenza A PR/8/1934 NS1 protein, a flexible GSG linker, amino acids 331-530 of SARS-COV-2 Wuhan-Hu-1 spike S1 protein, another GSG linker, and the PR8 nuclear export protein (NEP or NS2) ORF. The NS1 to RBD fusion protein was separated from NEP by the P2A peptide derived from porcine teschovirus-1 2A. During translation, the P2A site allows expression of the downstream NEP protein as a separate polypeptide by an unknown mechanism, thought to involve ribosome slippage. For example, the NEP protein may be represented by SEQ ID NO: 108. Thus, the required function of NEP was maintained and NS1 functionality should be preserved because the entire NS1 ORF was also maintained. Artificial segments 8 may be unstable due to at least 2 reasons. To improve the stability of the sequence two changes were implemented. When splicing was eliminated a portion of the segment that encodes NEP exon 1 and part of exon 2 had to be duplicated (see FIGS. 21 and 22). So, a number of silent mutations were introduced throughout both the NS1 and NEP ORFs to reduce the homology between these duplications. Additionally, both the GSG and the P2A site sequences were optimized to reflect the A-T rich codon bias of influenza. The SARS-COV-2 sequence was not changed as it was already >60% A-T.

It is possible that the NS1-RBD fusion may not perform the NS1 functions or the NS1 function may be impaired. If so, the recombinant virus may be deficient in ability to alter mRNA polyadenylation and splicing as well to repress both interferon and RIG-I mediated innate responses. To address this possibility, another construct with a cleavage site from those a asigna virus 2A (T2A) between the NS1 and the RBD was constructed (FIG. 2). This design was intended to allow expression of three separate polypeptides: NS1, RBD and NEP.

The vectors encoding new CoV2 NS segments were used in standard plasmid based influenza virus reverse genetics procedure to rescue the M2 deficient single replication (M2SR) viruses with SARS-COV-2 RBD segment 8. Both viruses were obtained successfully using the HA and NA segments from WHO-recommended vaccine strain of A/Singapore/INFIMH-16-0019/2016 IVR-186 (H3N2). Virus were recovered using M2VeroA cells that were engineered to constitutively express the M2 protein (SEQ ID NOs: 1, 15, 17) missing from M2SR grown in animal origin free (AOF) media. This virus rescue and culture system was appropriate for preparation of virus seed for cGMP production of M2SR vaccine candidate intended for testing in human clinical trial.

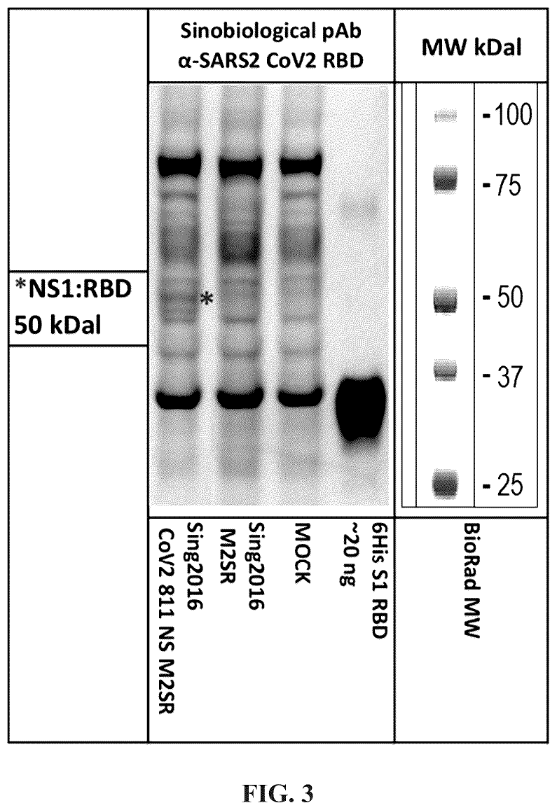

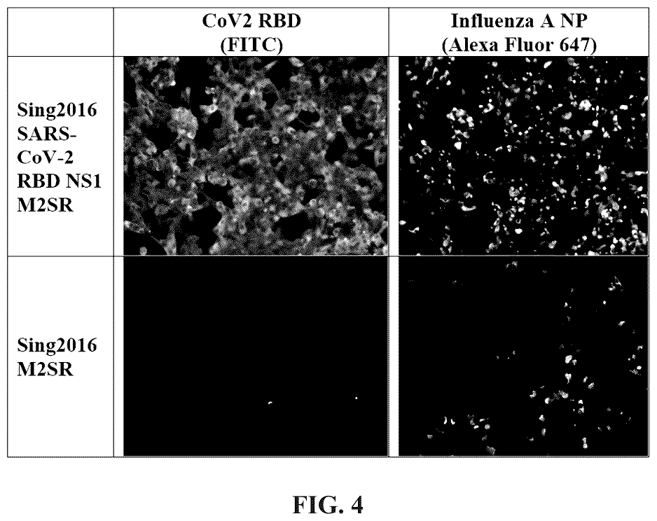

Expression of the NS1 SARS-COV-2 fusion construct was tested by infecting Vero cells with the CoV2 NS1 M2SR virus strain at high multiplicity of infection (MOI)>1.0. Both a MOCK no virus and Singapore 2016 M2SR with no RBD insert infections were also performed. Eleven hours after inoculation cells were harvested for immunoblot analysis of total cell lysates. Results indicate that antisera to SARS RBD binds a protein of the expected size. The band was only detected in the RBD virus infected cell extract but not in the controls (FIG. 3).

To confirm expression of the NS1 SARS-COV-2 fusion construct the same Vero cells were infected at high MOI and cells were fixed with formalin for immune-fluorescent staining. Cells were incubated with antisera to antisera to SARS RBD. After washing cells were stained by an anti-Rabbit Fluorescein Isothiocyanate (FITC) labeled secondary and an anti-influenza A NP antibody direct labeled by ALEXA FLOUR™ 647. Images shown in FIG. 4 show that both CoV-2 NS1 M2SR and the standard M2SR infected cells express detectable levels of the influenza A NP protein. Meanwhile the FITC labeling of the RBD could only be detected in CoV-2 NS1 M2SR infected cells which gave significant detectable fluorescence. The staining indicates that the NS1-RBD fusion protein is cytoplasmic.

### Example 3

This example demonstrates successful expression of SARS-COV-2 RBD from the influenza B BM2SR vector.

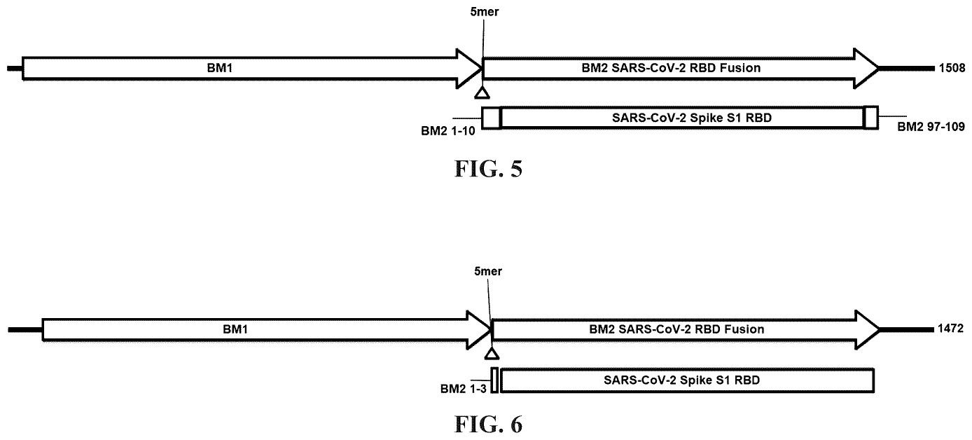

To express the SARS-COV-2 RBD antigen from the influenza B BM2SR vector engineered influenza BM2-deficient segment 7s were constructed synthetically (FIG. 5-6, SEQ ID NOs: 83, 84). The designed gene segments were then inserted into an RNA Pol I vector for expression as negative sense vRNA. The segment 7s were designed to express 2 polypeptide ORFs from within a single viral mRNA: first the complete influenza B/Florida/4/2006 M1 protein, a 5-mer ribosomal stop-start slippage site; and second a fusion protein of BM2 to amino acids 330-524 of SARS-COV-2 Wuhan-Hu-1 spike S1 protein. A 5 base (5-mer) sequence motif found naturally between BM1 and BM2, TTATG (SEQ ID NO: 20), contains both BM1 ORF translation stop codon (bold) and the start codon for BM2 ORF (italics). Ribosome slippage and re-initiation of translation allows the viral expression of BM2 in a second reading frame without need for splicing, in contrast to the influenza A segment 7. In the synthetic SARS-COV-2 RBD-containing influenza B segment 7 a small portion of the BM2 ORF was fused to the S1 RBD (SEQ ID NOs: 95, 96).

Artificial influenza segments may be unstable leading to poor virus growth in culture that is incompatible with manufacturing. This due to at least two reasons. First expression of an essential activity, in this case the M1 matrix protein may be impacted. To maintain the stability of the sequence the local RNA structure near the 5-mer was maintained so that M1 translation was not affected. Thus, amino terminal amino acids of BM2 ORF were fused to the SARS-COV-2 RBD. A segment may be lost due to low packaging efficiency. The termini of all influenza genomic segments are self-complementary so they can pair via hybridization, initiating formation of a complex tertiary structure dependent on sequences both untranslated and translated located up to 100 bp from both ends of the segment. Preservation of the correct UTR of the segment was paramount. In the vaccine segment 7 coronavirus sequence was fused to the influenza B 3′ UTR (mRNA sense). The UTR was longer than the UTR of influenza A at 85 bp long. Two versions were constructed. A more conservative version encodes insertion of the RBD into BM2 so that longer stretches from both ends of the BM2 ORF were preserved (FIG. 5, SEQ ID NO: 84). The longer version retains 10 and 13 terminal amino acids of BM2 respectively (FIG. 6, SEQ ID NO: 96).

A more trimmed down version only contains 9 bp, 3 residues of the N-terminus of BM2 fused to RBD (SEQ ID NO: 95) and then directly to the segment UTR (FIG. 6, SEQ ID NO: 83). The SARS-COV-2 sequences were examined to see if they reflect the A-T rich codon bias of influenza. The SARS-COV-2 sequence was not altered as it was already about 60% A-T.

The vectors encoding 2 SARS-COV-2 M segments (SEQ ID NOs: 83, 84) were used in standard plasmid-based influenza virus reverse genetics procedure to rescue the BM2-deficient single-replication (BM2SR) viruses containing SARS-COV-2 RBD BM2SR segment 7. Both viruses were obtained successfully using the HA and NA segments from WHO-recommended vaccine strain of B/CA/12/2015 (YL). Virus were recovered using BM2Vero cells that were engineered to constitutively express the BM2 protein (SEQ ID NOs: 2, 16, 18) missing from BM2SR virus grown in animal origin free (AOF) media. This virus rescue and culture system was appropriate for preparation of virus seed for cGMP production of BM2SR vaccine candidate intended for testing in human clinical trial.

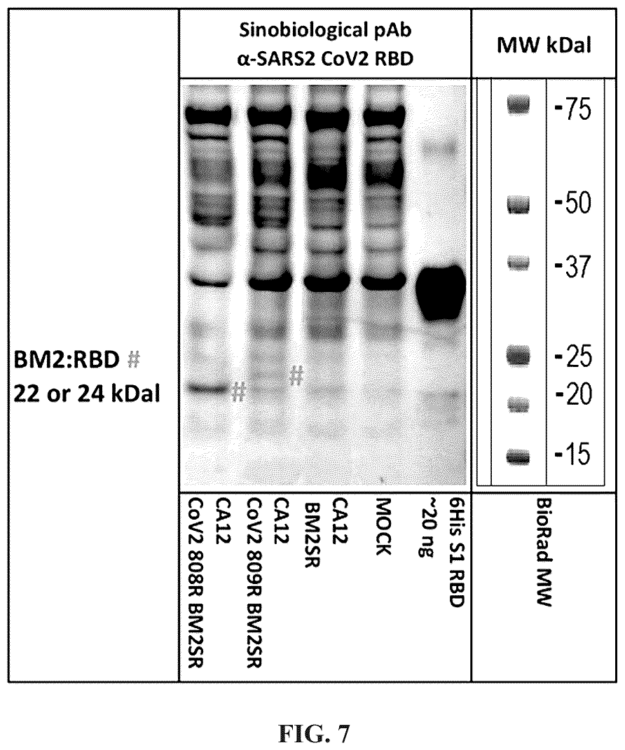

Expression of the SARS-COV-2 BM2 fusion protein constructs (SEQ ID NOs: 95, 96) were tested by infecting Vero cells with the CoV-2 BM2SR virus strain at high multiplicity of infection (MOI)>1.0. Both a MOCK no virus and vector only CA12 BM2SR with no RBD insert infections were also performed. Eleven hours after inoculation cells were harvested for immunoblot analysis of total cell lysates. Results indicate that antisera to SARS RBD binds a protein of the expected size. The bands were only detected in the RBD virus infected cell extracts and not in the control extracts (FIG. 7). These results suggest that the minimal 22 kDa RBD construct expresses to higher levels than the longer 24 kDa version.

### Example 4

This example demonstrates that M2SR and BM2SR viruses that encode sequences from SARS-COV-2 were attenuated in vivo.

Seven-week-old BALB/c, female mice were immunized intranasally with the following viral constructs as shown in Table 4. The M2SR and BM2SR backbone sequences and sequences for the segments encoding the SARS-COV-2 sequences are set forth in SEQ ID NOs: 43-47, 56, 58, 60, 63-67, 73, 80, 83, 95, 97 and 107. These viruses were administered at a dose of 1×10⁶ TCID₅₀ per mouse. A control group of mice was given DPBS, pH 7.2 containing 10% sucrose and 5 mM sodium glutamate (SPGNa). The mice were observed for 14 days after immunization for any change in body weight and symptoms of infection.

TABLE 4
Vaccine Groups

| Flu A | | | | |
|---|---|---|---|---|
| M2SR | cage | Prime antigen | Boost antigen | N |
| Mouse | 1 | AM2SR-CovidS-1 | AM2SR-CovidS-1 | 6 |
| exp. C1 | 2 | AM2SR-CovidS-1 | Covid-S1 protein IM | 6 |
| Group A | 5 | M2SR-Sing V5 (empty | M2SR-Sing V5 | 6 |
| | | vector) | | |
| | 6 | M2SR-Sing V5 (empty | Covid-S1 protein IM | 6 |
| | | vector) | | |
| | 9 | PBS/SPGNa | Covid-S1 protein IM | 3 |
| | 10 | PBS/SPGNa | PBS/SPGNa | 6 |
| Flu B | | | | |
| BM2SR | cage | Prime antigen | Boost antigen | n = |
| Mouse | 3 | BM2SR-CovidS-1 | BM2SR-CovidS-1 | 6 |
| exp. C1 | 4 | BM2SR-CovidS-1 | Covid-S1 protein IM | 6 |
| Group B | 7 | BM2SR-CA12 (empty | BM2SR-CA12 | 6 |
| | | vector) | | |
| | 8 | BM2SR-CA12 (empty | Covid-S1 protein IM | 6 |
| | | vector) | | |
| | 9 | PBS/SPGNa | Covid-S1 protein IM | 3 |
| | 10 | PBS/SPGNa | PBS/SPGNa | 6 |

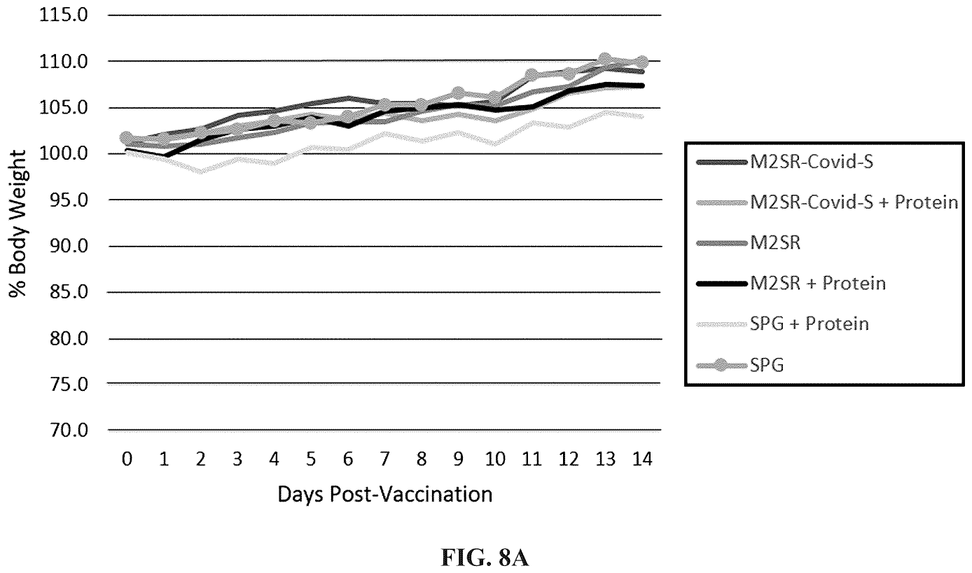

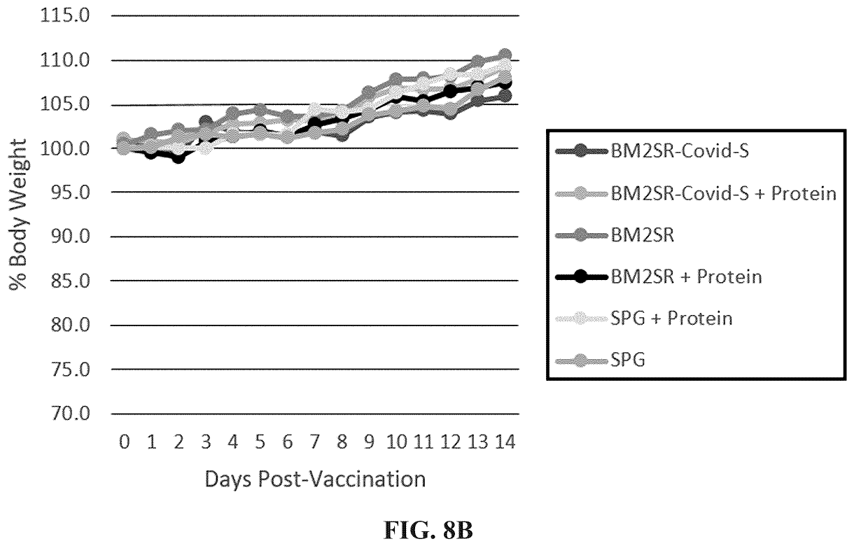

No clinical symptoms of infection or body weight loss were observed in mice immunized with the M2SR or BM2SR mutants or SPG control over the 14-day period. FIG. 8A depicts the mouse percent body weight change after immunization for the M2SR recombinant viruses and FIG. 8B for the BM2SR recombinant viruses. Moreover, the change in body weight between the groups was comparable over the 14-day period. These results indicate that the M2SR and BM2SR viruses that contain SARS-COV-2 sequences were attenuated and not pathogenic in mice.

### Example 5

This example demonstrates that M2SR and BM2SR viruses from Example 4 elicit antibody responses against SARS-COV-2.

Serum was collected from mice before prime immunization and about 3 weeks after the primary dose. Anti-spike RBD serum IgG antibody titers from the serum samples were pooled for each group were determined by enzyme-linked immunosorbent assay (ELISA).

The ELISA was performed using soluble SARS-COV-2 recombinant RBD protein with a C-terminal HIS-tag expressed in 293T cells and purified by using COMPLETE™ His-Tag Purification resin (F. Hoffmann-La Roche A G, Basel, Switzerland). The ELISA plates were coated overnight at 4 °C with 100 μL of the RBD protein at a concentration of 2 μg/mL in phosphate-buffered saline (PBS). After blocking the plate with PBS containing 0.1% polysorbate 20 (PBS-T) and 1% gelatin from cold water fish skin, the plates were incubated in duplicate with mouse serum diluted in PBS-T with 1% gelatin from cold water fish skin. After a two-hour incubation at room temperature, the plates were washed with PBS-T six times and then incubated with anti-mouse IgG secondary antibody conjugated with horseradish peroxidase (KPL; 1:2,000 dilution in PBS-T with 1% gelatin from cold water fish skin). After a one-hour incubation with the secondary antibody, the plates were washed six times with PBS-T and then developed with 1-STEP™ Ultra TMB-ELISA Substrate Solution (Thermo Fisher Scientific, Waltham, MA). After a ten-minute incubation, the reaction was stopped with the addition of 4N sulfuric acid. The absorbance was measured at a wavelength of 450 nm (OD₄₅₀). Endpoint titers were the reciprocal of the dilution that was above the cut-off value determined by subtracting the mean value of the blanks plus 6×standard deviations.

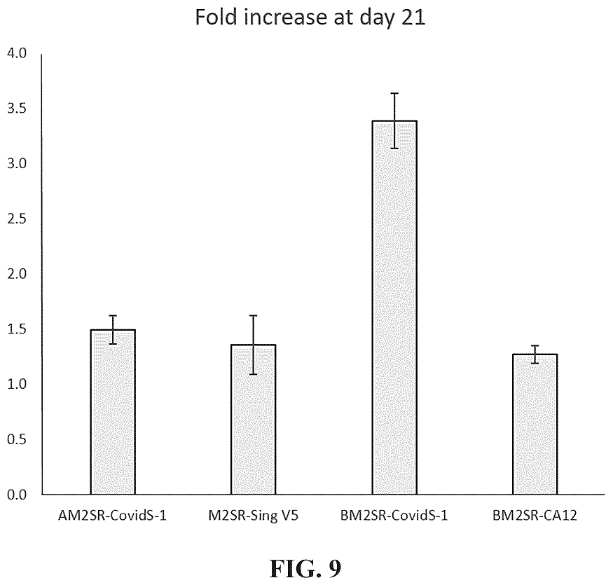

The fold increase in ELISA titer from pre-immunization baseline is shown in FIG. 9. The empty vectors that did not encode spike RBD sequences did not show an increase in ELISA titer on day 21. The M2SR and BM2SR viruses that encoded SARS-COV-2 sequences elicited an increase in RBD spike ELISA titer.

### Example 6

This example demonstrates that systemic antibodies are generated after a second dose of vaccine used for prime or spike protein.

Four dosing regimens of viruses in Example 4 were assessed: (1) mice primed with one of the M2SR-COVID-19 vaccine candidates (i.e., AM2SR-CovidS-1) or M2SR vector virus (i.e., M2SR-Sing V5) and then boosted with the same (i.e., AM2SR-CovidS-1 or M2SR-Sing V5) intranasally approximately 4 weeks post prime, (2) mice primed with one of the M2SR-COVID-19 vaccine candidates (i.e., AM2SR-CovidS-1) or M2SR vector virus (i.e., M2SR-Sing V5) intranasally and boosted about 4 weeks post prime with purified SARS-COV-2 protein intramuscularly, (3) mice primed with one of the BM2SR-COVID-19 vaccine candidates (i.e., BM2SR-CovidS-1) or BM2SR vector viruses (i.e., BM2SR-CA12) and then boosted with the same intranasally approximately 4 weeks post prime, and (4) mice primed with one of the BM2SR-COVID-19 vaccine candidates (i.e., BM2SR-CovidS-1) or BM2SR vector viruses (i.e., BM2SR-CA12) intranasally and boosted about 4 weeks post prime with purified SARS-COV-2 protein intramuscularly. All mice were terminally bled then euthanized about 3 weeks post-secondary immunization (boost) and serum samples were collected for analysis.

Serum samples were analyzed by ELISA as described above with respect to Example 5.

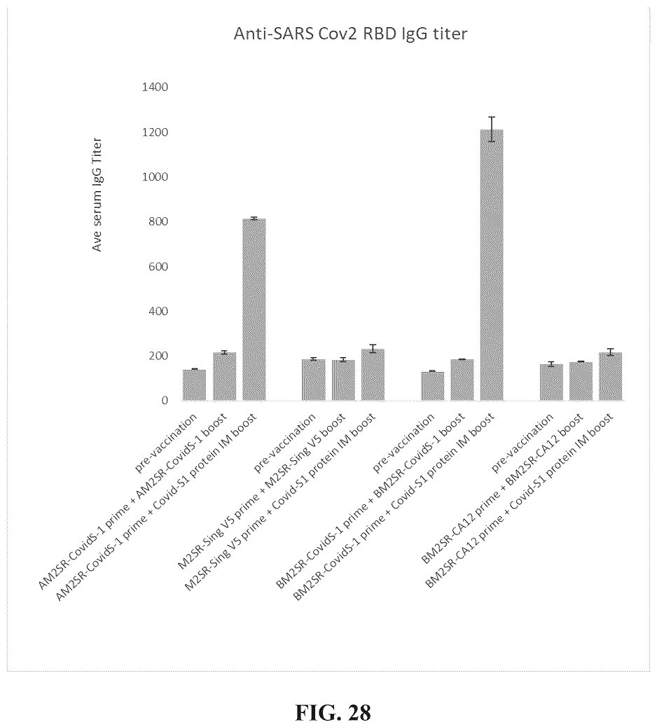

The anti-SARS-COV-2 RBD IgG titers are shown in FIG. 28. The empty vectors that do not encode spike RBD sequences (i.e., M2SR-Sing V5 and BM2SR-CA12) administered twice (prime-boost) did not elicit an increase in RBD spike ELISA titer. The M2SR and BM2SR viruses that encoded SARS-COV-2 sequences (i.e., AM2SR-CovidS-1 and BM2SR-CovidS-1) administered twice (prime-boost) elicited an increase in RBD spike ELISA titer. Priming with M2SR and BM2SR viruses that encoded SARS-COV-2 sequences (i.e., AM2SR-CovidS-1 and BM2SR-CovidS-1) substantially increased the RBD spike ELISA titer when boosted with purified SARS-COV-2 protein whereas priming with empty M2SR vector viruses did not elicit a substantial increase in RBD spike ELISA titer when boosted with purified SARS-COV-2.

### Example 7

This example demonstrates that the M2SR and BM2SR vectors can be used in multivalent formulations and retain immunogenicity to each.

Influenza A H1N1 or H3N2 FGHY1-M2SR or BM2SR-Vic or BM2SR-Yam viruses elicit antibody responses when formulated as a monovalent, bivalent, trivalent, or quadrivalent vaccine.

Seven-week-old BALB/c female mice (N=8) were immunized intranasally with monovalent H1N1 FGHY1-M2SR, monovalent H3N2 FGHY1-M2SR, bivalent H1N1 and H3N2 FGHY1-M2MR, monovalent BM2SR-Victoria, monovalent BM2SR-Yamagata, bivalent BM2SR, trivalent H1N1 and H3N2 FGHY1-M2SR and BM2SR Victoria or Yamagata, or quadrivalent H1N1 and H3N2 FGHY1-M2SR and BM2SR Victoria and Yamagata vaccines. A control group of mice were mock immunized with SPG. At 28 days after vaccination, the mice were immunized intranasally with a boost immunization consisting of the same vaccine administered for the prime immunization. Serum samples were taken on days 7, 14, and 21 following prime immunization and on days 35, 42 and 49 following the boost immunization (day 28). Anti-H1 HA, anti-H3 HA, anti-influenza B-Vic HA and anti-influenza B-Yam HA serum IgG antibody titers from the serum samples were determined by ELISA.

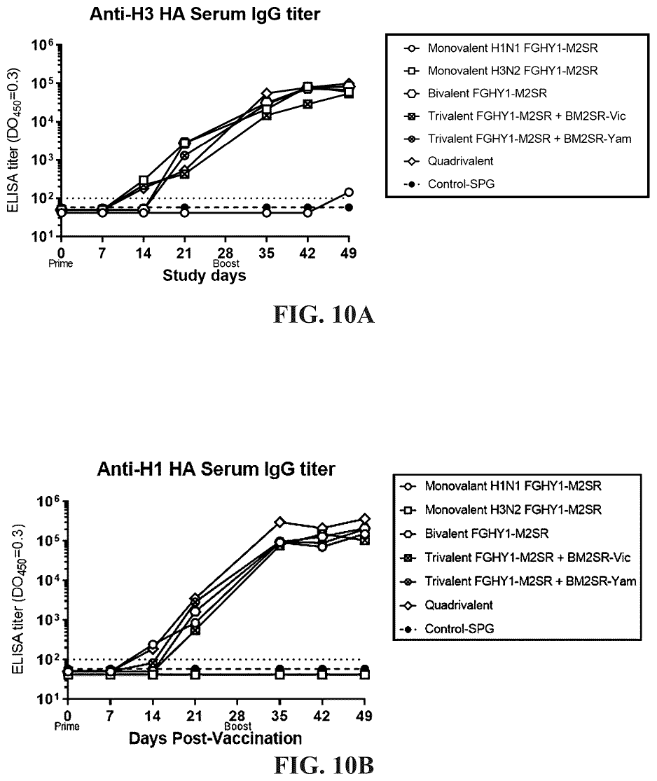

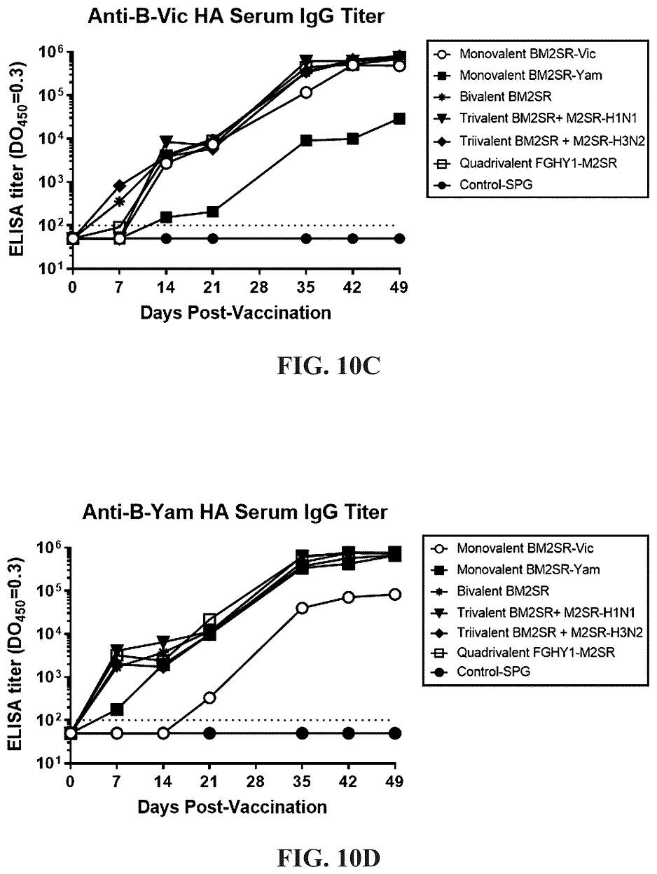

The resulting anti-H1 HA data is shown in FIG. 10A. The resulting anti-H3 HA data is shown in FIG. 10B. The resulting anti-influenza B-Vic HA data is shown in FIG. 10C. The resulting anti-influenza B-Yam HA data is shown in FIG. 10D. The results demonstrated that all vaccines were able to elevate anti-influenza virus antibodies above SPG control and that these increases were comparable across vaccine formulations. Further, these results demonstrate that the monovalent components maintain ability to elicit immune responses to the monovalent components when formulated into multivalent vaccines.

### Example 8

This example is expected to demonstrate that the M2SR-SARS-COV-2 vaccine elicits antibody responses in vivo that were increased upon repeat dosing with no toxicity to the host.

To demonstrate that the M2SR-SARS-COV-2 vaccine virus elicits immune responses against the component without causing toxicity to the host, 15 male and 15 female ferrets will be immunized intranasally with the M2SR-SARS-COV-2 vaccine at a dose level of 1×10⁸ TCID₅₀ (low dose) or 1×10⁹ TCID₅₀ (high dose). A third group of ferrets will be mock immunized intranasally with SPG as a placebo control. A three-dose vaccination regimen will be utilized for each treatment group. Ferrets will be administered the prime immunization (study day 1) and the 2 boost immunizations 13 and 27 days later (study days 14 and 28). Following each immunization, ferrets will be observed for 7 days for mortality, with body weights, body temperatures and clinical signs measured daily. Blood will be collected to assess clinical pathology pre-study and on study days 14, 16, 30, and 49 from all surviving ferrets. Serum samples will be collected pre-study and on study days 14, 30, and 49 to evaluate antibody levels over time by ELISA, hemagglutination inhibition (HA1) assay, and virus neutralization (VN) assay. Necropsy will be performed on 5 males and 5 females per group on study days 3, 30, and 49, including examination of the external surface of the body, all orifices, the cranial, thoracic and peritoneal cavities, and their contents.

Vaccine Virus Immunization. Ferrets will be immunized intranasally with three doses of an M2SR-SARS-COV-2 vaccine at either a dose of 1×10⁸ TCID₅₀ or a dose of 1×10⁹ TCID₅₀. Vials of frozen vaccine virus stock will be thawed at room temperature for at least 10 minutes and then stored refrigerated, or on wet ice, until use. Ferrets will be anesthetized with ketamine/xylazine and the virus dose administered intranasally in a volume of 500 μL (250 μL per nare).

The M2SR-SARS-COV-2 vaccine virus is a recombinant influenza A virus that does not express a functional M2 protein, encoding the HA and NA genes of influenza A/Singapore/INFIMH-16-0019/2016 (H3N2) and the RBD of the SARS-COV-2 spike protein.

Experimental Design: Ninety (90) ferrets (Triple F Farms, Sayre, PA), 45 males and 45 females, 16 to 22 weeks old at the time of study initiation, will be utilized for the study. All animal procedures will be performed in an animal biosafety level 2 facility in accordance with the protocols approved by the animal care and use committee at IIT Research Institute. Prior to immunization, ferrets will be monitored for 4 days to establish baseline body temperatures. Temperature readings will be recorded daily through a transponder (BioMedic data systems, Seaford, DE) implanted subcutaneously in each ferret. Blood will be collected prior to study initiation, and serum tested for influenza antibodies. Pre-immunization serum samples will be treated with receptor destroying enzyme (RDE) (Denka Seiken, Tokyo, Japan) to remove nonspecific inhibitors, then serially diluted, tested against a defined amount of influenza A/Michigan/45/2015 (H1N1), A/Singapore/INFIMH-16-0019/2016 (H3N2), B/Phuket/3073/2013 (Yamagata lineage), and B/Colorado/06/2017 (Victoria lineage) viruses and mixed with 0.5% turkey red blood cells. Antibody titers will be defined by the lowest serum dilution causing inhibition of red blood cell agglutination. Only ferrets with HA1 titers less than 40 will be considered seronegative and used in this study. Study animals will be randomized and divided into 3 groups (15 male and 15 female ferrets/group).

To assess the vaccine efficacy and toxicity, ferrets will be immunized intranasally with three doses of 1×10⁸ TCID₅₀ or three doses of 1×10⁹ TCID₅₀ of the M2SR-SARS-COV-2 on study days 1, 14, and 28. The control group will be mock immunized intranasally with SPG on study days 1, 14, and 28. Ferret body temperatures, weights, and clinical symptoms will be monitored daily for 7 days post-immunizations. Blood will be collected to assess clinical pathology on study days −5, 14, 16, 30, and 49 from all surviving ferrets. Serum samples will be collected on study days −5, 14, 30, and 49 and kept frozen at approximately −70 °C until measurement of antibody titer by ELISA, virus neutralization assay and HA1 assay. All study animals will be euthanized on their scheduled dates (day 3, 30, or 49, 5 males and 5 females per group) and necropsied. Necropsy consists of examination of the external surface of the body, all orifices, and the cranial, thoracic and peritoneal cavities and their contents. The tissues will be collected, fixed, and evaluated histopathologically by a board-certified veterinary pathologist.

Moribundity/Mortality and Clinical Observations: All ferrets are expected to survive until their scheduled date of sacrifice. All ferrets are expected to have an activity level score of “0” (alert and playful) for all time points measured during Days 1-49.

Body Weights and Body Weight Changes: No differences are expected to be observed.

Body Temperatures: No differences are expected to be observed.

Enzyme-linked immunosorbent assay (ELISA): Anti-HA IgG antibody titers from serum samples will be determined by ELISA. ELISA plates will be coated by recombinant HA protein from A/Singapore/INFIMH-16-0019/2016 (H3N2) (Immune Technology Corp., New York, NY) or spike RBD, blocked by skim milk, and samples were applied. Ferret IgG antibodies were detected by horseradish peroxidase labeled anti-ferret IgG goat antibodies (SeraCare Life Sciences, Milford, MA) and 1-STEP™ Ultra TMB-ELISA (Thermo Fisher Scientific Inc.) substrate.

Ferrets in each of the immunized groups are expected to show significant elevation of anti-H3 HA antibody in serum, while antibody levels in animals that received SPG only are not expected to change from baseline. Anti-H3 HA antibody titers will be higher in immunized groups than SPG control groups two weeks after the prime dose. Mean antibody titers per immunized group will be increased further following first and second administrations of the vaccine.

Hemagglutination Inhibition (HA1) Assay: To demonstrate the functional activity of antibodies detected by ELISA, serum samples will be analyzed by HA1 assay. Serum samples will be treated with RDE to eliminate inhibitors of nonspecific hemagglutination. RDE will be reconstituted per the manufacturer's instructions. Serum will be diluted 1:3 in RDE and incubated 18-20 hours in a 37 °C±2 °C water bath. After the addition of an equal volume of 2.5% (v/v) sodium citrate, the samples will be incubated in a 56±2 °C water bath for 30=5 minutes. A solution comprised of 0.85% NaCl will be added to each sample to a final serum dilution of 1:10 after the RDE treatment. The samples will then be further diluted two-fold (1:10 to 1:1,280) in PBS and incubated with 4 hemagglutinating units of influenza A/Singapore/INFIMH-16-0019/2016 (H3N2) viruses. After incubation, 0.5% avian red blood cells will be added to each sample and incubated for 30±5 minutes. Presence or absence of hemagglutination will then be scored.

High dose (1×10⁹ TCID₅₀) groups are expected to show higher HA1 titer than low dose (1×10⁸ TCID₅₀) groups, and the SPG (control) group is not expected to elicit any HA1 titers. M2SR-SARS-COV-2 immunized ferrets are expected to demonstrate equal to or higher than 80 HA1 titers against test virus. The CDC states that serum HA1 antibody titers of 40 were associated with at least a 50% reduction in risk for influenza infection or disease in populations. Therefore, these results are expected to show that M2SR-SARS-COV-2 virus is able to elicit protective immune responses.

Virus Neutralization Assay: Pre-study and treatment phase serum samples (from study days 3, 14, 30, and 49) will be tested against A/Singapore/INFIMH-16-0019/2016 (H3N2) influenza virus in a virus neutralization assay. The serum samples will be inactivated at 56 °C for 30 minutes. The sera then will be serially diluted 2-fold and incubated with standardized virus (concentration of 80-140 PFU) at 37±2 °C in 5.0±1% CO₂ for 60 minutes. One hundred microliter (100 μL) of each serum and virus mixture will then be transferred into the respective wells of a 96-well plate containing a monolayer of MDCK cells. The plate (with samples) will then be incubated for 18-22 hours at 37±2 °C in 5.0±1% CO₂. After incubation, the cells will be fixed with paraformaldehyde and stained with an anti-influenza A nucleoprotein monoclonal antibody pool (1 part MAB8257: 1 part MAB8258 (Millipore; Billerica, MA)) followed by peroxidase-conjugated goat anti-mouse IgG. The spots will be developed using TrueBlue Peroxidase Substrate (Kirkegaard and Perry Laboratories, Gaithersburg, MD). The plaques will be visualized and counted using an enzyme-linked immunospot (ELISPOT™) instrument (AID GmbH, Strassberg, Germany). The 50% plaque reduction neutralization titer (PRNT₅₀) will be calculated by counting plaques and reporting the titer as the reciprocal of the last serum dilution to show 50% reduction of the input control virus plaque count as based on the back-titration of control plaques.

Ferrets within the SPG group are expected to remain negative (titers≤100) for the duration of the study. Ferrets who were immunized with M2SR-SARS-COV-2 at a dose of 1×10⁸ TCID₅₀ are expected to have high geometric mean titers (GMT). Ferrets who were immunized with M2SR-SARS-COV-2 at a dose of 1×10⁹ TCID₅₀ are expected to have higher GMT VN titers. All ferrets immunized with 3 doses of H3N2 M2SR-SARS-COV-2 at 1×10⁹ TCID₅₀ are expected to exhibit the highest PRNT₅₀ titers.

Clinical Pathology: For all surviving ferrets, blood samples for the analysis of clinical chemistry, hematology and coagulation parameters will be collected from the jugular vein or vena cava pre-study and on days 3, 14, 16, 30, and 49. Animals will be fasted for 4-6 hours prior to blood collection. Ethylenediaminetetraacetic acid (EDTA) will be used as the anticoagulant for hematology samples, while sodium citrate will be used for coagulation samples. Samples for clinical chemistry will be collected without an anticoagulant. Urine samples will be collected directly from the bladder of each ferret at necropsy.

No treatment-related or toxicologically significant findings are expected to be noted for any of the clinical chemistry or hematology parameters evaluated during the study.

Gross Necropsy and Histopathology: Gross necropsy and histopathology will be carried out on study days 3, 30, and 49 for 5 males and 5 females per group. Intranasal immunization of M2SR-SARS-COV-2 to ferrets at a dose of 1×10⁸ TCID₅₀ is expected to result in no gross findings. At a dose of 1×10⁹ TCID₅₀, gross findings are expected to be noted in the lung (pigmentation, dark or mottled), and microscopic findings are expected to be noted in the lung (mixed cell infiltrates) on Day 3 and 30. After a 3-week recovery, on study day 49, no test article-related gross lesions are expected to be observed.

This example is expected to show that intranasal immunization of the M2SR-SARS-COV-2 vaccine virus does not spread in the vaccinated host and is not associated with any vaccine-related adverse events (e.g., elevated body temperature, loss of weight, or clinical signs). These results are expected to indicate that the M2SR-SARS-COV-2 virus elicits protective immune responses against homologous test virus after a single dose that can be further elevated with repeat dosing and is useful as an intranasal coronavirus vaccine.

### Example 9

This example demonstrates the successful design and generation of several M2SR virus strains that express a variety of antigens derived from SARS-COV-2 spike protein.

Influenza A NS segment 8 that encodes two non-structural proteins NS1 and NEP (nuclear export protein) can be modified to use influenza A as a vaccine vector to express antigens. NEP is required, while the NS1 ORF can be dispensable for viral replication. NS1 truncations can be isolated by repeated passage in Vero cell culture and even complete NS1 deletion strains can be constructed. NS1 does play very important roles in promoting influenza infection including alterations to splicing and blocking host cell innate response. NS1 mutations reduce viral titer making manufacture difficult, and more importantly they impair viral replication in primary cells and in vivo. A second major obstacle to vectorizing the NS segment is that the essential NEP protein is expressed from a spliced form of segment 8 mRNA. Splicing joins a short exon 1 sequence to an exon 2 in an alternate translational reading frame that overlaps with NS1 ORF coded for by unspliced mRNA.

To encode an antigen two important changes are made to the NS segment. First, splice donor (SEQ ID NO: 109) and acceptor sites are abolished. Then the sequences encoding NEP exon 1 and exon 2 are joined to construct the NEP ORF without intron. The result is an ORF encoding a single polypeptide wherein NS1 C-terminus is fused to NEP ORF separated by NS1 P2A peptide derived from porcine teschovirus-1 2A and a GSG flexible linker (SEQ ID NOs: 80, 85, 87-91, 97-104). During translation, the P2A site allows expression of NEP protein as a separate polypeptide by an unknown mechanism, thought to involve ribosome slippage. Genetic information encoding a desired vaccine antigen is inserted between the influenza ORFs, expressed either by fusion to NS1 or by addition of a second P2A or T2A peptide to cleave both sides of the antigen (SEQ ID NO: 86, 99). This arrangement results in an enlarged segment 8 carrying long repeats of the nucleotide sequences from within the NS1 ORF.

Unfortunately, such duplications are genetically unstable. Atypical hybridization between two portions of the segment during genome replication may induce influenza RNA polymerase errors typically leading to deletions and insertions and defective genomic RNA synthesis. The instability may be exacerbated in this case because the gene duplication includes NEP exon 1 sequences only 26 bp from the segment 8 terminus that are likely to important for genome packaging. Assembly of influenza genomic segments into the virion is mediated via the double stranded “pan handle” RNA structure formed by hybridization of inverted repeat sequences in the 5′ and 3′ UTR. Coding sequences near segment ends are also involved in packaging and silent mutations that are near segment termini have been shown to block viral replication. The duplicated region internal to the segment could compete for essential hybridization with the terminal UTR packaging sequences impairing virus assembly. The result is poor virus growth, low viral titer, and loss of transgene expression.

To improve genetic stability by reducing homology between tandem duplications in the engineered segment, key mutations were introduced to sequences encoding both the NS1 and NEP ORFs (SEQ ID NOs: 110, 111). Silent mutations were made to 3rd positions of codons encoding NS1 to cut potential for unintended expression by eliminating start codons and adding stop codons to any potential alternate translation reading frames. This reduces the chance for production of unintended neoantigens from both vector and insert sequences or both.

The exon 1 sequence duplication coding for the 10 N-terminal amino acids of NEP was significantly altered in two ways to reduce sequence identity between copies (FIG. 21, SEQ ID NOs: 80, 97, 110). The NEP is expressed by this construct via P2A cleavage site. Although the mechanism is not understood cleavage always leaves a single prolyl residue as a protein scar appended to the N terminus of the cleaved downstream peptide. The 3rd amino acid of NEP is already proline, suggesting these first 2 residues are not structurally relevant. Thus, the constructed NS segment was designed to express a 6 bp deletion N terminal mutant of NEP that begins at proline with no scar (SEQ ID NO: 117). The deleterious homology was further ameliorated by changing codon third positions yet maintaining high % A-T. Additionally, both the GSG and the P2A site sequences were codon optimized to reflect the A-T rich codon bias of influenza.

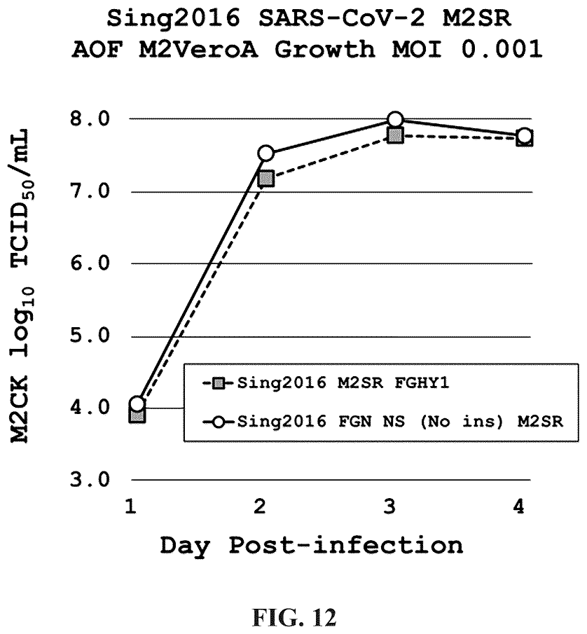

The splice negative NS segment containing the duplication (SEQ ID NO: 85, 98) was inserted into RNA Pol I plasmid vector for expression as negative sense vRNA. Standard plasmid-based influenza virus reverse genetics procedure was used to rescue M2 deficient single replication (M2SR) virus containing the engineered and control A PR/8/1934 segments 8. Recovered virus strains were amplified in M2VeroA cells and viral titer was determined. The strains were used to inoculate triplicate cultures at MOI=0.001 for comparison of growth kinetics. The four days following inoculation viral cultures were sampled and aliquots were stored frozen for later titration analysis. Following determination of virus titer by TCID₅₀, daily mean titers were computed. A plot (FIG. 12) of the curves for the two strains show that virus growth is not impaired by the synthetic segment expressing NS1 and NEP as a single self-cleaving peptide.

Several M2SR virus strains were generated that are designed to express a variety of antigens derived from SARS-COV-2 spike protein were grown in M2VeroA cells as given in Table 5.

TABLE 5
M2SR SARS CoV-2 Vaccine Candidate Strains and Maximum Viral Titer

| | | | SARS-CoV-2 S1 | | |
|---|---|---|---|---|---|
| | | MAX | Position | | |
| Description | Vector | Titer | Start | End | Length |
| WT | PR8 M2SR, PR8 NS | 7.91 | N/A | N/A | N/A |
| | (unmodified) | | | | |
| | (SEQ ID NO: 107, 60) | | | | |
| A PR8 NS P2A no | PR8 NS1 P2A NEP (SEQ ID | 8.14 | N/A | N/A | N/A |
| insert | NOs: 85, 98) | | | | |
| A PR8 NS CoV-2 | PR8 NS1 Fusion P2A NEP | 7.81^{a} | 331 | 530 | 200 |
| RBD fusion | (SEQ ID NOs: 80, 97) | | | | |
| CoV-2 RBD | PR8 NS1 T2A CoV2 P2A NEP | 7.74 | 330 | 530 | 201 |
| | (SEQ ID NO: 99) | | | | |
| NS1-CoV2 NTD | PR8 NS1 Fusion P2A NEP | <0.67^{b} | 26 | 295 | 270 |
| fusion | (SEQ ID NOs: 87, 100) | | | | |
| NS1-CoV-2 NoP | PR8 NS1 Fusion P2A NEP | 6.67 | 942 | 1031 | 90 |
| HELIX fusion | (SEQ ID NOs: 88, 101) | | | | |
| NS1 CoV-2 2P | PR8 NS1 Fusion P2A NEP | 7.67 | 942 | 1031 | 90 |
| HELIX fusion | (SEQ ID NOs: 89, 102) | | | | |
| NS1 CoV-2 | PR8 NS1 Fusion P2A NEP | 7.67 | 1073 | 1139 | 67 |
| Connector fusion | (SEQ ID NOs: 90, 103) | | | | |
| NS1 CoV-2 HR2 | PR8 NS1 Fusion P2A NEP | 7.69 | 1140 | 1213 | 74 |
| fusion | (SEQ ID NOs: 91, 104) | | | | |
| CoV-2 MHC RBD | PR8 M2SR M2 Fusion | 7.19 | 365 | 382 | 18 |
| 5.1 M2 fusion | (SEQ ID NOs: 10, 55) | | | | |
| CoV-2 MHC RBD | PR8 M2SR M2 Fusion | 7.50 | 440 | 466 | 27 |
| 2.X M2 fusion | (SEQ ID NOs: 8, 105) | | | | |
| CoV-2 MHC TM1.1 | PR8 M2SR M2 Fusion (SEQ ID | 8.08 | 1198 | 1228 | 31 |
| M2 fusion | NOs: 6, 106) | | | | |
| CoV-2 RBD M2 | PR8 M2SR Replacement M2 | <0.67^{b} | | | |
| fusion | Exon 2 (SEQ ID NOs: 79, 92) | | | | |
| CoV-2 RBD M1 | PR8 M2SR splice negative with | <0.67^{b} | | | |
| fusion | M1 fusion to S1 RBD with P2A | | | | |
| | site (SEQ ID NOs: 81, 93) | | | | |
| CoV-2 RBD M2 | PR8 M2SR splice negative with | <0.67^{b} | | | |
| fusion | M1 fusion to S1 RBD with | | | | |
| | pentamer site (SEQ ID NOs: 82, | | | | |
| | 94) | | | | |

^{a}= TCID₅₀ performed in Vero cells
^{b}= Assay limit of Detection

### Example 10

This example demonstrates that the functionality of the NS vector segment to express an antigen that is the spike helix of the SARS-COV-2 S1 protein.

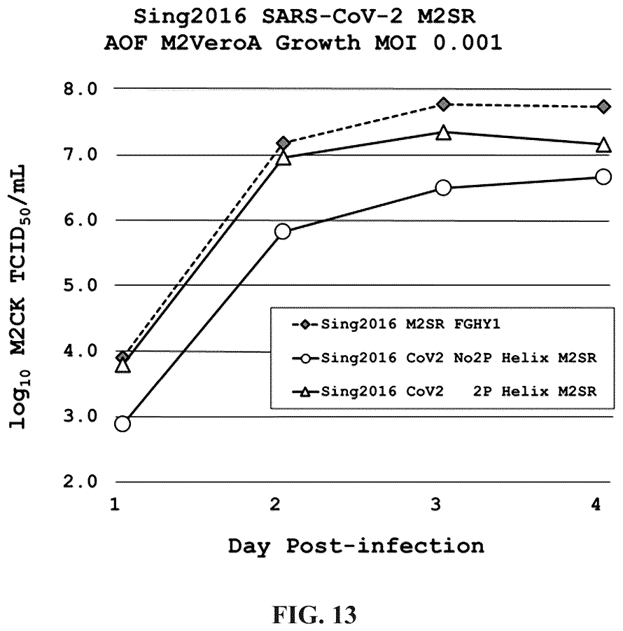

The spike helix of the SARS-COV-2 S1 protein is known to undergo a large conformational change that drives fusion between viral and cell membranes. Study of other viral spike protein helices including those of RSV and PIV has identified a set of 2 tandem proline mutations that improve performance by stabilizing the spike proteins as recombinant antigens. These two proline residues (2P) are in a turn between two shorter helices that exist in the spike protein pre-fusion conformation. This change locks the protein in pre-fusion conformation which results in dual benefits of far better recombinant protein expression and in neutralizing immunologic response to vaccination. The growth curve in FIG. 13 shows that segment 8 with NS1 fusion to unmodified SARS-COV-2 helix antigen impairs the virus growth (SEQ ID NOs: 88, 101) as compared to wild-type. Replacement of the turn residues with 2P to lock the spike helix into prefusion form improves growth (SEQ ID NOs: 88, 102), perhaps by improving NS1 functionality.

### Example 11

This example demonstrates successful expression of SARS-COV-2 receptor binding domain (RBD) from the influenza A M2SR segment.

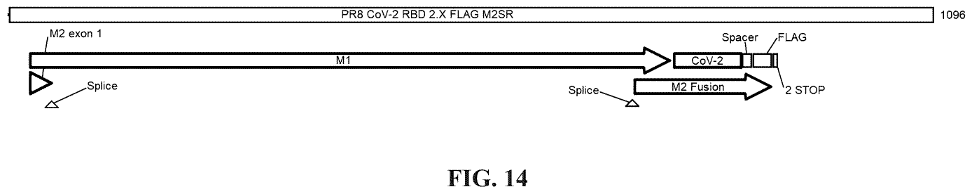

Various SARS-COV-2 RBD antigens were expressed from engineered influenza A M2SR influenza A M2-deficient vector segments 7 (SEQ ID NOs: 122-124) that were constructed synthetically (FIG. 14). The designed gene segments were then inserted into an RNA Pol I vector for expression as negative sense vRNA. The segment 7 is designed to express 2 polypeptide open reading frames (ORFs) from a spliced viral mRNA: first the complete influenza A/PR/8/34 M1 protein, and second a fusion protein of M2 to antigens of SARS-COV-2 Wuhan-Hu-1 spike S1 protein. The viral expression of M2 in a second reading frame of the influenza A segment 7 occurs by splicing. To maintain essential M1 protein function from the synthetic SARS-COV-2 containing influenza A segment 7 a portion of the M2 ORF is fused to the S1 RBD (SEQ ID NOs: 6, 8, 10, 79).

The vectors encoding SARS-COV-2 M2SR segments were used in standard plasmid-based influenza virus reverse genetics procedure to rescue the M2-deficient single-replication (M2SR) viruses containing SARS-COV-2 RBD M2SR segment 7. Both viruses were obtained successfully using the HA and NA segments from WHO-recommended vaccine strain of A/Singapore//2016 (H3N2). Virus were recovered using M2Vero cells that are engineered to constitutively express the M2 protein missing from M2SR virus grown in AOF media. This virus rescue and culture system is appropriate for preparation of virus seed for cGMP production of M2SR vaccine candidate intended for testing in human clinical trial.

### Example 12

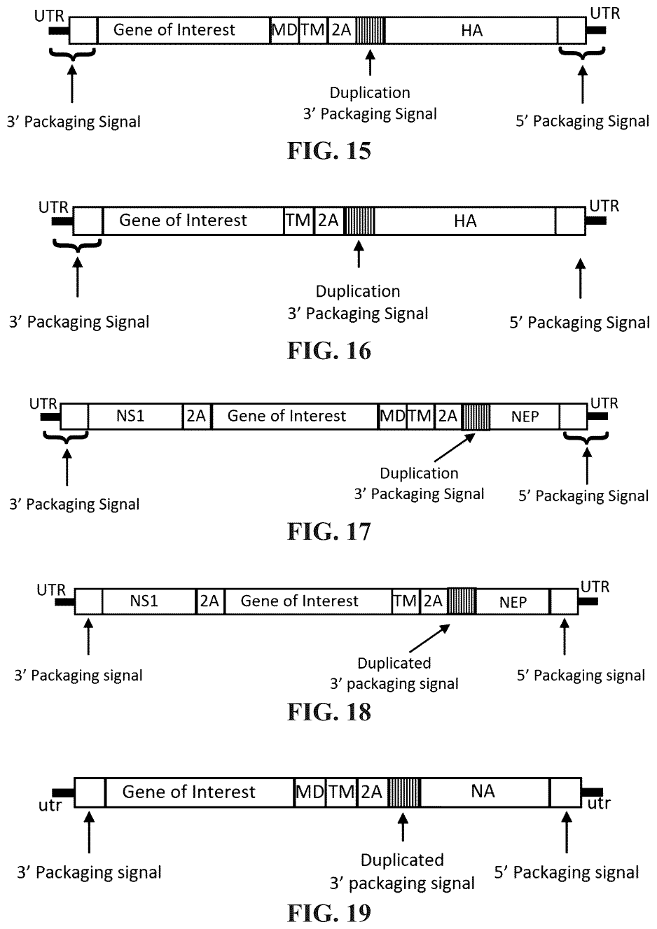

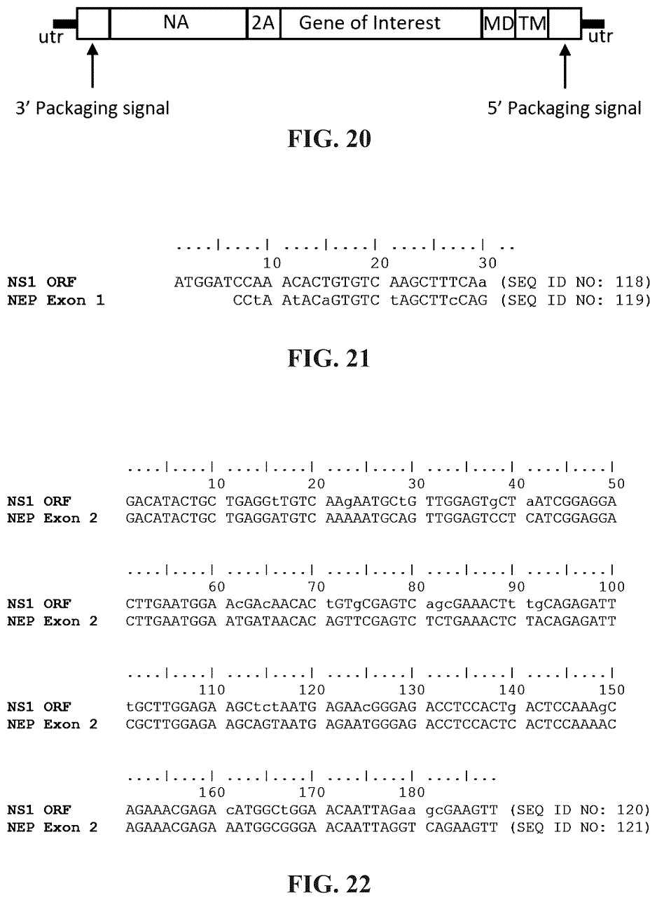

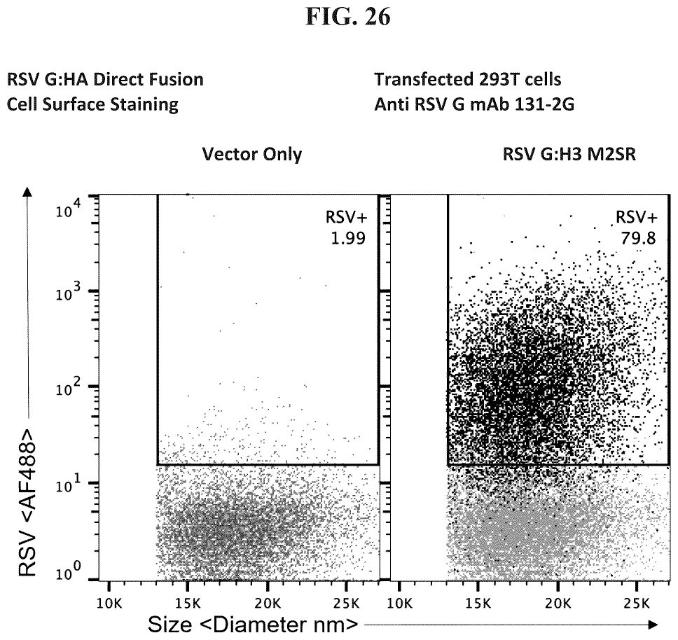

This example demonstrates that M2SR influenza virus is capable of driving expression of antigen anchored to the extracellular membrane of infected cells (see FIGS. 15-20 and 24-27).

Packaged virus produced in supportive M2 expressing substrate cells were anticipated to substantially lack protein encoded by the gene of interest, multimerization domain, or transmembrane domain, unless the antigen was directly fused with an influenza subunit that is incorporated, such as hemagglutinin, HA. The gene of interest (GOI) can be antigenically relevant elements of viral (including influenza), bacterial, fungal, or protozoal origin.

Proper expression of the antigen on the cell surface can be confirmed by immune fluorescence staining analyzed by flow cytometry of infected cells with monoclonal antibodies specific for an intended vaccine antigen encoded by virus. Different segments can be used to express the same antigen. An example antigen is SARS-COV-2 Spike protein human ACE2 receptor binding domain (RBD). The RBD antigen can be expressed at cell surface using TM domains from another membrane protein. Fusion of RBD to the TM from respiratory syncytial virus (RSV) F protein was encoded by the NS1 segment from a single open reading frame (ORF) that contains P2A and T2A translational slippage sites that produce three peptides: NS1, NEP and the RBD-RSV™ fusion with T4 trimer domain. An alternate approach is fusion directly to the HA protein itself. HA is a membrane protein so fused antigen will be displayed as a trimer on the surface of infected cells and incorporated into the virion.

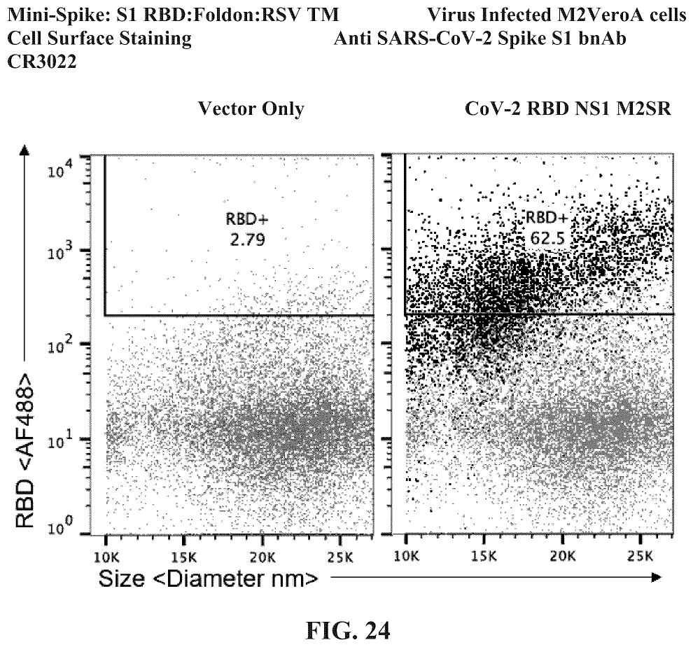

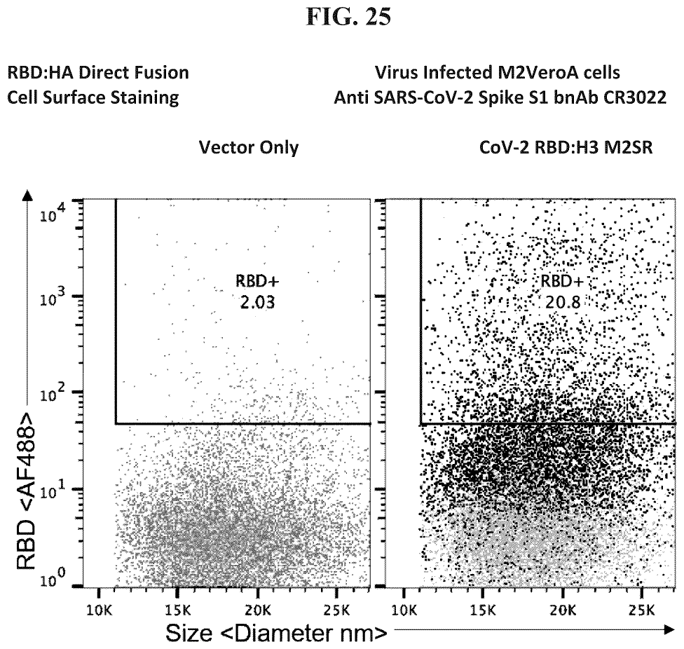

M2VeroA cells were inoculated at multiplicity of infection (MOI) between 1 to 10 by M2SR viruses expressing RBD at the membrane from either the NS1 segment or the HA segment. Infected cells were immune stained for surface expression of SARS-COV-2 S1 RBD at 18-hours post-inoculation in FACS buffer (1×DPBS, 1% FBS) at 4 °C. Live intact cells were stained using as primary a neutralizing monoclonal antibody CR3022 isolated from convalescent SARS-COV-1 patient (ter Meulen et al., PLOS Med. 3(7): e237 (2006)) followed by detection with Alexa Fluor 488-labeled anti-human IgG secondary antibody as seen in FIGS. 24-25. Surface expression above background was detected from cells infected with virus expressing SARS-CoV-2 RBD antigen from either the NS or the HA segments.

Another respiratory virus that can be targeted is respiratory syncytial virus (RSV). The two major surface proteins of RSV, Fusion (F) and surface glycoprotein (G), are both important binding sites for monoclonal antibodies that can neutralize RSV. Human 293T cells were chemically transfected by influenza A replicon of 4 DNA plasmids to constitutively over express PA, PB1, PB2 and NP viral subunits required to express proteins encoded by an influenza RNA segment; and by a single plasmid expressing influenza HA genomic segment from RNA polymerase I promoter. The RSV G protein antigen was directly fused to the HA glycoprotein leading to expression of RSV G antigen on cell membrane. Live, intact cells were immune stained 48 hours post-transfection for surface expression of RSV G surface glycoprotein antigen at 48-hours post-transfection. Staining was in FACS buffer using primary mouse monoclonal antibody 131-2G (Chemicon) followed by detection with Alexa Fluor 488-labeled anti-mouse IgG secondary antibody. Surface expression above background was detected from cells infected with virus expressing RSV G protein antigen.

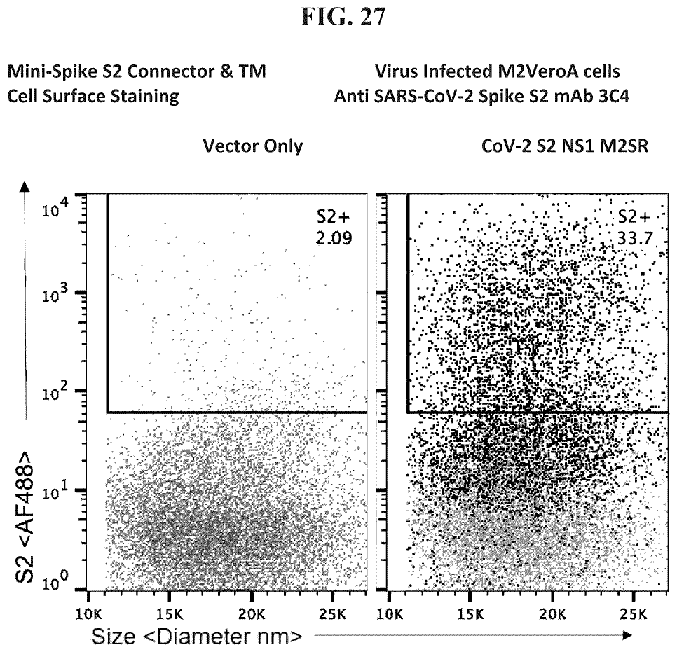

Multiple antigens from a single pathogen may be displayed on the membrane. Cells inoculated at MOI=1 with M2SR virus encoding SARS-COV-2 S2 antigen express second alternate COVID vaccine target other than RBD on the surface as shown in FIG. 27. Intact live M2VeroA cells infected by virus at MOI=1 were immune stained for surface expression of SARS-COV-2 spike S2 subunit antigen at 18-hours post-inoculation. Staining was in FACS buffer using primary rabbit monoclonal antibody 3C4 (Genscript) raised against SARS-COV-2 S2 immunogen followed by detection with Alexa Fluor 488-labeled anti-rabbit IgG secondary antibody. Surface expression above background was detected from cells infected with virus expressing SARS-COV-2 S2 connector and TM antigen.

Packaging signals are maintained upstream of the GOI for constructs for both HA and NS segments, and in the case of HA, the packaging signal can be duplicated to maintain proper HA processing. The duplicated packaging signal shall have silent mutations to eliminate its secondary interaction with the 5′ packaging signal and help prevent undesired recombination events. Other embodiments may employ direct fusion of the antigen (e.g., sequences from Respiratory Syncytial Virus (RSV) Fusion (F) or Glycoprotein (G) or SARS-COV2 spike sequences) to HA itself, in which case the packing sequence is not duplicated (FIGS. 24-27, SEQ ID NOs: 116-118). In many cases, the use of a multimerization domain (MD) would be preferred to enhance immunogenicity. Such a MD can be selected from a variety of motifs such as C-terminal domain of T4 fibritin (SEQ ID NO: 115, Foldon) or GCN4-p1 leucine zipper domain. The transmembrane domain (TM) can be that of the SARS-COV2 spike predicted transmembrane helix amino acid 1214 through 1246 (Genebank Accession: YP_009724390.1, SEQ ID NOs: 36-41, 77, 119), the use of at least amino acids 1201 to 1246 would a priori include the top ranked MHC I compatible epitope in Table 1 (SEQ ID NO: 21). Other™ can include that of RSV fusion protein (SEQ ID NO: 115)

Table 6 shows mini spike proteins that are portions of SARS-COV-2 S1 protein or fusions of portions of S1 designed to be membrane anchored using the Spike™. Use of the S1 signal sequence (SEQ ID NOs: 39-41, 42, 115, 119) will direct the peptide to the cellular secretory apparatus for display on the cell surface membrane and for post-translational modifications such as N-linked glycosylation.

TABLE 6
Mini Spike Proteins Including Portions of SARS-CoV-2 S1 Protein

SEQ ID NO: 36
167 AA Protein-Mini Spike No Head Spike S2 protein 1069 to 1235
PAQEKNFTTAPAICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNESLIDLQELGKYEQ
YIKWPWYIWLGFIAGLIAIVMVTIMLC
SEQ ID NO: 37
Protein-Mini Spike No 2P Helix Spike S2 protein 942 to 1235
ASALGKLQDVVNQNAQALNTLVKQLSSNFGAISSVLNDILSRLDKVEAEVQIDRLITGRLQSLQTYVTQQ
LIRAAEIRASANLAATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVYDPLQPELDSFKEE
LDKYFKNHTSPDVDLGDISGINASVVNIQKEIDRLNEVAKNLNESLIDLQELGKYEQYIKWPWYIWLGFI
AGLIAIVMVTIMLC
SEQ ID NO: 38
Protein-Mini Spike 2P Helix Spike S2 protein 942 to 1235
ASALGKLQDVVNQNAQALNTLVKQLSSNFGAISSVLNDILSRLDPPEAEVQIDRLITGRLQSLQTYVTQQ
LIRAAEIRASANLAATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVTGIVNNTVYDPLQPELDSFKEE
LDKYFKNHTSPDVDLGDISGINASVVNIQKEIDRLNEVAKNLNESLIDLQELGKYEQYIKWPWYIWLGFI
AGLIAIVMVTIMLC
Bold = 2 P Mutation
SEQ ID NO: 39
Protein-Mini Spike 2P Helix with Signal Peptide
Spike protein 1 to 26, 942 to 1235
MFVFLVLLPLVSSQCVNLTTRTQLPPASALGKLQDVVNQNAQALNTLVKQLSSNFGAISSVLNDILSRLD
PPEAEVQIDRLITGRLQSLQTYVTQQLIRAAEIRASANLAATKMSECVLGQSKRVDFCGKGYHLMSFPQS
APHGVVFLHVTYVPAQEKNFTTAPAICHDGKAHFPREGVFVSNGTHWFVTQRNFYEPQIITTDNTFVSGN
CDVVIGIVNNTVYDPLQPELDSFKEELDKYFKNHTSPDVDLGDISGINASVVNIQKEIDRLNEVAKNLNE
SLIDLQELGKYEQYIKWPWYIWLGFIAGLIAIVMVTIMLC
Bold = 2 P Mutation
Underline = S1 signal peptide
SEQ ID NO: 40
Protein-Mini Spike NTD with Signal Peptide Spike protein 1 to 294,
1069 to 1235
MFVFLVLLPLVSSQCVNLTTRTQLPPAYTNSFTRGVYYPDKVFRSSVLHSTQDLFLPFFSNVTWFHAIHV
SGTNGTKRFDNPVLPFNDGVYFASTEKSNIIRGWIFGTTLDSKTQSLLIVNNATNVVIKVCEFQFCNDPF
LGVYYHKNNKSWMESEFRVYSSANNCTFEYVSQPFLMDLEGKQGNFKNLREFVFKNIDGYFKIYSKHTPI
NLVRDLPQGFSALEPLVDLPIGINITRFQTLLALHRSYLTPGDSSSGWTAGAAAYYVGYLQPRTFLLKYN
ENGTITDAVDCALDPALQPELDSFKEELDKYFKNHTSPDVDLGDISGINASVVNIQKEIDRLNEVAKNLN
ESLIDLQELGKYEQYIKWPWYIWLGFIAGLIAIVMVTIMLC
Underline = S1 signal peptide
Underline Italics = S1 NTD
SEQ ID NO: 41
Protein-Mini Spike RBD with Signal Peptide Spike protein 1 to 26,
331 to 530, 1069 to 1235
MFVFLVLLPLVSSQCVNLTTRTQLPPNITNLCPFGEVFNATRFASVYAWNRKRISNCVADYSVLYNSASF
STFKCYGVSPTKLNDLCFTNVYADSFVIRGDEVRQIAPGQTGKIADYNYKLPDDFTGCVIAWNSNNLDSK
VGGNYNYLYRLFRKSNLKPFERDISTEIYQAGSTPCNGVEGFNCYFPLQSYGFQPTNGVGYQPYRVVVLS
FELLHAPATVCGPKKSTYVPAQEKNFTTAPAICHDGKAHFPREGVFVSNGTHWFVTQRNFYEPQUITTDN
TFVSGNCDVVIGIVNNTVYDPLQPELDSFKEELDKYFKNHTSPDVDLGDISGINASVVNIQKEIDRLNEV
AKNLNESLIDLQELGKYEQYIKWPWYIWLGFIAGLIAIVMVTIMLC
Underline = S1 signal peptide
BOLD = S1 RBD
SEQ ID NO: 115
Protein-Mini Spike RBD with Mini-Signal Peptide, Trimerization
Domain, and RSV TM Spike S1 protein 2 to 14, 331 to 530,
T4 fibritin 457 to 454 (Foldon), RSV F 521 to 552
FVFLVLLPLVSSQPNITNLCPFGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIAWNSNNLDSKVGGNYNYLYRLF
RKSNLKPFERDISTEIYQAGSTPCNGVEGFNCYFPLQSYGFQPTNGVGYQPYRVVVLSFELLHAPATVCG
PKKSGYIPEAPRDGQAYVRKDGEWVLLSTFLGSTTNIMITTIIIVIIVILLSLIAVGLLLYCKAR
Underline = S1 signal peptide
BOLD = S1 RBD
Italics = T4 Foldon
Underline Italics = RSV TM
SEQ ID NO. 119
Protein:-Mini Spike with SARS-CoV-2 S protein signal
peptide 2 to 26 and S2 helical connector domain to TM
1069 to 1235 with GSG linkers, N-terminal T2A, C and
P2A site.
GSGEGRGSLLTCGDVEENPGPFVFLVLLPLVSSQCVNLTTRTQLPPAQEKNFTTAPAICHDGKAHFPREG
VFVSNGTHWFVTQRNFYEPQIITTDNTFVSGNCDVVIGIVNNTVYDPLQPELDSFKEELDKYFKNHTSPD
VDLGDISGINASVVNIQKEIDRLNEVAKNLNESLIDLQELGKYEQYIKWPWYIWLGFIAGLIAIVMVTIM
LCGSGATNFSLLKQAGDVEENPGP
BOLD: GSG linker
Italics: T2A site
Underline: P2A site
BOLD Underline = S1 signal peptide

### Example 13

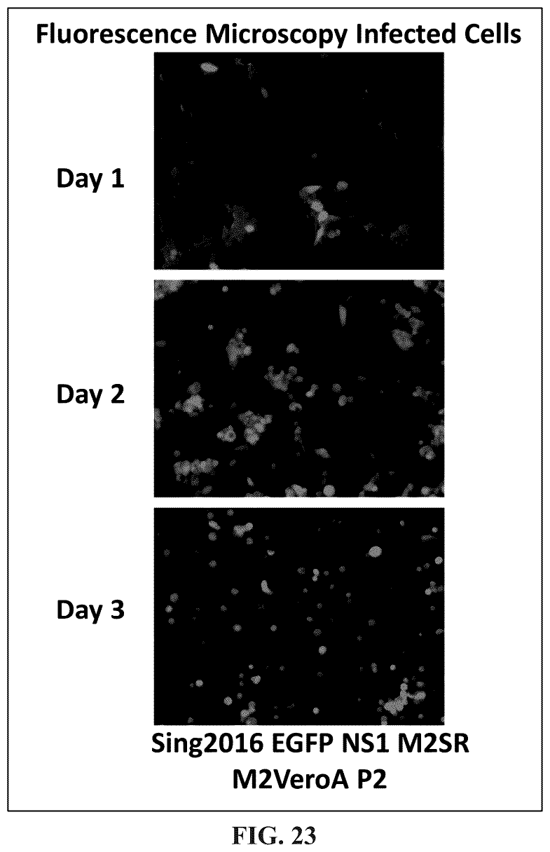

This example demonstrates that the functionality of the NS vector segment of M2SR containing silent mutations (FIGS. 21-22) to express an antigen (SEQ ID NO: 113) that is also a marker gene (SEQ ID NO: 114), the EGFP fluorescent protein. M2SR virus containing NS segment designed to express EGFP was used to infect M2VeroA cells at MOI=10. Fluorescence microscopy over a three-day period post-inoculation showed that robust EGFP expression could be detected within 24 hours, spreading until nearly 100% of cells were seen to express the antigen at 48 hours. By 72 hours, cells detached from the substrate and exhibited strong cytopathic effect (CPE), as expected due to influenza infection (FIG. 23). Strong CPE that was observed at Day 3 shows that the virus replication was not substantially inhibited by the inserted EGFP gene.

### Example 14

This example demonstrates that M2SR and BM2SR viruses encoding SARS-COV-2 sequences from Example 4 retain the ability to elicit antibody responses against the influenza HA (hemagglutinin) surface protein.

Serum was collected from mice before prime immunization and about 3 weeks after the primary dose. Serum samples were pooled for each group and anti-HA IgG antibody titers were determined by enzyme-linked immunosorbent assay (ELISA).

The ELISA was performed using recombinant HA protein as the capture antigen. Recombinant H3 HA (ATM) (A/Singapore/INFIMH-16-0019/2006) (H3N2) [Immune-Tech, New York, NY] was used for serum ELISA analysis of mice administered M2SR vector virus or M2SR virus that encode SARS-COV-2 sequences. Recombinant InfB HA1 (B/Phuket/3073/2013) [Immune-Tech, New York, NY] was used for serum ELISA analysis of mice administered BM2SR vector virus or BM2SR virus that encode SARS-COV-2 sequences.

The ELISA plates were coated overnight at 4 °C with 100 μL of the capture antigen at a concentration of 2 μg/mL in phosphate-buffered saline (PBS). After blocking the plate with PBS containing 0.1% polysorbate 20 (PBS-T) and 1% gelatin from cold water fish skin, the plates were incubated in duplicate with mouse serum diluted in PBS-T with 1% gelatin from cold water fish skin. After a two-hour incubation at room temperature, the plates were washed with PBS-T six times and then incubated with anti-mouse IgG secondary antibody conjugated with horseradish peroxidase (KPL; 1:2,000 dilution in PBS-T with 1% gelatin from cold water fish skin). After a one-hour incubation with the secondary antibody, the plates were washed six times with PBS-T and then developed with 1-STEP™ Ultra TMB-ELISA Substrate Solution (Thermo Fisher Scientific, Waltham, MA). After a ten-minute incubation, the reaction was stopped with the addition of 4N sulfuric acid. The absorbance was measured at a wavelength of 450 nm (OD₄₅₀). Endpoint titers were the reciprocal of the dilution that was above the cut-off value determined by subtracting the mean value of the blanks plus 0.3 OD₄₅₀.

No difference in serum anti-HA IgG levels were observed between the M2SR and BM2SR viruses encoding SARS-COV-2 sequences and their corresponding vector virus as shown in Table 7.

TABLE 7
Anti-HA Titer Levels

| Prime antigen | Anti- HA Titer |
|---|---|
| AM2SR-CovidS-1 | >12,500 |
| M2SR-Sing V5 | >12,500 |
| PBS/SPGNa | <100 |
| BM2SR-CovidS-1 | >12,500 |
| BM2SR-CA12 | >12,500 |
| PBS/SPGNa | <100 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Sequencing conventions are based on DNA referring to the four nucleotides: adenine (A), guanine (G), cytosine (C), and thymine (T). When referring to RNA or influenza virus, T means uracil (U).

1. A recombinant virus comprising an influenza viral backbone, wherein the influenza viral backbone comprises PB1, PB2, PA, NP, M, NS, HA, and NA gene segments comprises at least one nucleotide sequence that encodes one or more antigens, wherein
(a) the PA gene segment comprises a thymine at nucleotide position 2272;
(b) the NP gene segment encodes a NP protein having an amino acid sequence comprising selected amino acids, wherein the selected amino acids comprise a serine at position 40, an asparagine or glycine at position 161, a threonine at position 204, and optionally a valine at position 93; and
(c) the NS gene segment comprises a guanine at nucleotide position 39, and wherein the NS gene segment encodes an NS protein having an amino acid sequence comprising selected amino acids, wherein the selected amino acids comprise a glutamine at position 176.

2. The recombinant virus of claim 1, wherein the antigen is an immunogenic fragment of SARS-COV-2 spike glycoprotein.

3. The recombinant virus of claim 1, wherein the M gene segment comprises at least one nucleotide sequence that encodes an antigen, wherein the antigen is an immunogenic fragment of SARS-COV-2 spike glycoprotein and further encodes a mutated BM2 protein.

4. The recombinant virus of claim 1, wherein the NS gene segment comprises at least one nucleotide sequence that encodes one or more antigens.

5. The recombinant virus of claim 1 wherein the antigen is an immunogenic fragment of SARS-COV-2 spike glycoprotein.

6. The recombinant virus of claim 1, wherein the NS gene segment encodes (1) an NS1 protein, (2) at least one flexible linker protein, (3) an immunogenic fragment of SARS-CoV-2 spike glycoprotein, (4) at least one cleavable cleavage sequence, and (5) a NEP protein.

7. The recombinant virus of claim 6, wherein the at least one cleavable cleavage sequence is a T2A peptide sequence or a P2A peptide sequence.

8. The recombinant virus of claim 1, wherein the NS gene segment encodes a protein comprising any one of SEQ ID NOs: 97-104.

9. The recombinant virus of claim 1, wherein each of the M and NS gene segments comprises at least one nucleotide sequence that encodes one or more antigens, and wherein the antigens are immunogenic fragments of SARS-COV-2 spike glycoprotein.

10. The recombinant virus of claim 1, wherein each of the NA and NS gene segments comprises at least one nucleotide sequence that encodes one or more antigens, and wherein the antigens are immunogenic fragments of SARS-COV-2 glycoprotein.

11. The recombinant virus of claim 1, wherein each of the M and NA gene segments comprises at least one nucleotide sequence that encodes one or more antigens, and wherein the antigens are immunogenic fragments of SARS-COV-2 glycoprotein.

12. The recombinant virus of claim 1, wherein each of the M and HA gene segments comprises at least one nucleotide sequence that encodes one or more antigens, and wherein the antigens are immunogenic fragments of SARS-COV-2 glycoprotein.

13. The recombinant virus of claim 1, wherein each of the NS and NA gene segments comprises at least one nucleotide sequence that encodes one or more antigens, and wherein the antigens are immunogenic fragments of SARS-COV-2 glycoprotein.

14. The recombinant virus of claim 1, wherein each of the NS and HA gene segments comprises at least one nucleotide sequence that encodes one or more antigens, and wherein the antigens are immunogenic fragments of SARS-COV-2 spike glycoprotein.

15. The recombinant virus of claim 1, wherein the virus is capable of replication in human cells.

16. The recombinant virus of claim 1, wherein the virus has enhanced growth as compared to a recombinant virus that is the same except without the selected amino acids in Vero cells under the same conditions.

17. The recombinant virus of claim 1, wherein the gene segment that comprises at least one nucleotide sequence that encodes one or more antigens further comprises a downstream duplication and wherein the downstream duplication comprises at least one silent nucleotide mutation.

18. A pharmaceutical formulation comprising the recombinant virus of claim 1.

19. The pharmaceutical formulation of claim 18, wherein the vaccine is formulated as a monovalent vaccine, a bivalent vaccine, a trivalent vaccine, or a quadrivalent vaccine.

20. A method of eliciting an immune response in a mammal, the method comprising administering the recombinant virus of claim to the mammal, thereby eliciting an immune response to the antigen in the mammal.

21. The method of claim 20, wherein the mammal is a human.


SEQUENCE LISTING (119 sequences)

SEQ ID NO: 1 - protein, 97 aa, synthetic construct
  REGION 1..97: note = Synthetic Sequence
MSLLTEVETPIRNEWGCRCNGSSDPLTIAANIIGILHLTLWILDRLFFKCIYRRFKYGLKGGPSTEGVPKSMREEYRKEQQSAVDADDGHFVSIELE

SEQ ID NO: 2 - protein, 109 aa, synthetic construct
  REGION 1..109: note = Synthetic Sequence
MLEPLQILSICSFILSALHFMAWTIGHLNQIRRGVNLKIQIRNPNKEAINREVSILRHNYQKEIQAKETMKKILSDNMEVLGDHIVVEGLSTDEIIKMGETVLEVEELQ

SEQ ID NO: 3 - protein, 25 aa, synthetic construct
  REGION 1..25: note = Synthetic Sequence
MSLLTEVETPIRNEWGCRCNGSSDP

SEQ ID NO: 4 - protein, 37 aa, synthetic construct
  REGION 1..37: note = Synthetic Sequence
MSLLTEVETPIRNEWGCRCNGSSDPAGAGDYKDDDDK

SEQ ID NO: 5 - protein, 56 aa, synthetic construct
  REGION 1..56: note = Synthetic Sequence
MSLLTEVETPIRNEWGCRCNGSSDPIDLQELGKYEQYIKWPWYIWLGFIAGLIAIV

SEQ ID NO: 6 - protein, 68 aa, synthetic construct
  REGION 1..68: note = Synthetic Sequence
MSLLTEVETPIRNEWGCRCNGSSDPIDLQELGKYEQYIKWPWYIWLGFIAGLIAIVAGAGDYKDDDDK

SEQ ID NO: 7 - protein, 52 aa, synthetic construct
  REGION 1..52: note = Synthetic Sequence
MSLLTEVETPIRNEWGCRCNGSSDPNLDSKVGGNYNYLYRLFRKSNLKPFER

SEQ ID NO: 8 - protein, 64 aa, synthetic construct
  REGION 1..64: note = Synthetic Sequence
MSLLTEVETPIRNEWGCRCNGSSDPNLDSKVGGNYNYLYRLFRKSNLKPFERAGAGDYKDDDDK

SEQ ID NO: 9 - protein, 43 aa, synthetic construct
  REGION 1..43: note = Synthetic Sequence
MSLLTEVETPIRNEWGCRCNGSSDPYSVLYNSASFSTFKCYGV

SEQ ID NO: 10 - protein, 55 aa, synthetic construct
  REGION 1..55: note = Synthetic Sequence
MSLLTEVETPIRNEWGCRCNGSSDPYSVLYNSASFSTFKCYGVAGAGDYKDDDDK

SEQ ID NO: 11 - protein, 205 aa, synthetic construct
  REGION 1..205: note = Synthetic Sequence
MSLLTEVETPIRNEWGCRCNGSSDPLPPLLTDEMIAQYTSALLMNDNLDSKVGGNYNYLYRLFRKSNLKPFERWNWSPRRARSVASQSIIAYSNWAALQIPFAMQMAYRFNGIGVSWYSVLYNSASFSTFKCYGVSWGEVFNATRFASVYAWNRKRIGWMSLGAENSVAYSNNSIAIYFWQELGKYEQYIKWPWYIWLGFIAGLI

SEQ ID NO: 12 - protein, 217 aa, synthetic construct
  REGION 1..217: note = Synthetic Sequence
MSLLTEVETPIRNEWGCRCNGSSDPLPPLLTDEMIAQYTSALLMNDNLDSKVGGNYNYLYRLFRKSNLKPFERWNWSPRRARSVASQSIIAYSNWAALQIPFAMQMAYRFNGIGVSWYSVLYNSASFSTFKCYGVSWGEVFNATRFASVYAWNRKRIGWMSLGAENSVAYSNNSIAIYFWQELGKYEQYIKWPWYIWLGFIAGLIAGAGDYKDDDDK

SEQ ID NO: 13 - protein, 205 aa, synthetic construct; identical to SEQ ID NO: 11
  REGION 1..205: note = Synthetic Sequence

SEQ ID NO: 14 - protein, 217 aa, synthetic construct; identical to SEQ ID NO: 12
  REGION 1..217: note = Synthetic Sequence

SEQ ID NO: 15 - DNA, 294 bp, synthetic construct
  misc_feature 1..294: note = Synthetic Sequence
  source 1..294: mol_type = other DNA
atgagtcttctaaccgaggtcgaaacgcctatcagaaacgaatgggggtgcagatgcaacggttcaagtgatcctctcactattgccgcaaatatcattgggatcttgcacttgacattgtggattcttgatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaa

SEQ ID NO: 16 - DNA, 330 bp, synthetic construct
  misc_feature 1..330: note = Synthetic Sequence
  source 1..330: mol_type = other DNA
atgctcgaaccacttcagattctttcaatttgttctttcattttatcagctctccatttcatggcttggacaatagggcatttgaatcaaataagaagaggggtaaacctgaaaatacaaataaggaatccaaataaggaggcaataaacagagaggtgtcaattctgagacacaattaccaaaaggaaatccaagccaaagaaacaatgaagaaaatactctctgacaacatggaagtattgggtgaccacatagtagttgaagggctttcaactgatgagataataaaaatgggtgaaacagttttggaggtggaagaattgcaataa

SEQ ID NO: 17 - DNA, 294 bp, synthetic construct
  misc_feature 1..294: note = Synthetic Sequence
  source 1..294: mol_type = other DNA
atgtccctgctgaccgaagtggaaactcctattagaaacgagtggggctgtagatgtaacggctcaagcgaccctctgaccattgctgccaacatcattggcatcctgcacctgaccctgtggattctggaccgactgttctttaagtgcatctaccggagattcaagtatggactgaaaggaggaccaagcacagagggagtgcctaaatccatgagggaggaataccgcaaagagcagcagagcgccgtggacgcagatgatggacatttcgtgagcattgaactggaatga

SEQ ID NO: 18 - DNA, 330 bp, synthetic construct
  misc_feature 1..330: note = Synthetic Sequence
  source 1..330: mol_type = other DNA
atgctggaaccactgcagatcctgagtatttgctcttttatcctgagcgcactgcactttatggcctggactatcgggcacctgaaccagatcagaaggggcgtgaacctgaagatccagatcagaaacccaaacaaggaggccatcaaccgcgaagtgagcatcctgagacacaattaccagaaggagatccaggctaaagaaaccatgaagaaaatcctgtctgacaatatggaggtgctgggcgatcacatcgtggtggaaggactgagcaccgacgaaatcatcaaaatgggcgagactgtcctggaagtggaagaactgcagtaa

SEQ ID NO: 19 - protein, 9 aa, synthetic construct
  REGION 1..9: note = Synthetic Sequence
  VARIANT 5: note = X can be any naturally occurring amino acid
GDVEXNPGP

SEQ ID NO: 20 - skipped

SEQ ID NO: 21 - protein, 25 aa, synthetic construct
  REGION 1..25: note = Synthetic Sequence
QELGKYEQYIKWPWYIWLGFIAGLI

SEQ ID NO: 22 - protein, 19 aa, synthetic construct
  REGION 1..19: note = Synthetic Sequence
NLDSKVGGNYNYLYRLFRK

SEQ ID NO: 23 - protein, 14 aa, synthetic construct
  REGION 1..14: note = Synthetic Sequence
YRLFRKSNLKPFER

SEQ ID NO: 24 - protein, 27 aa, synthetic construct
  REGION 1..27: note = Synthetic Sequence
NLDSKVGGNYNYLYRLFRKSNLKPFER

SEQ ID NO: 25 - protein, 20 aa, synthetic construct
  REGION 1..20: note = Synthetic Sequence
AALQIPFAMQMAYRFNGIGV

SEQ ID NO: 26 - protein, 19 aa, synthetic construct
  REGION 1..19: note = Synthetic Sequence
EVFNATRFASVYAWNRKRI

SEQ ID NO: 27 - protein, 9 aa, synthetic construct
  REGION 1..9: note = Synthetic Sequence
GEVFNATRF

SEQ ID NO: 28 - protein, 20 aa, synthetic construct
  REGION 1..20: note = Synthetic Sequence
GEVFNATRFASVYAWNRKRI

SEQ ID NO: 29 - protein, 18 aa, synthetic construct
  REGION 1..18: note = Synthetic Sequence
YSVLYNSASFSTFKCYGV

SEQ ID NO: 30 - protein, 16 aa, synthetic construct
  REGION 1..16: note = Synthetic Sequence
SPRRARSVASQSIIAY

SEQ ID NO: 31 - protein, 18 aa, synthetic construct
  REGION 1..18: note = Synthetic Sequence
MSLGAENSVAYSNNSIAI

SEQ ID NO: 32 - protein, 35 aa, synthetic construct
  REGION 1..35: note = Synthetic Sequence
SPRRARSVASQSIIAYTMSLGAENSVAYSNNSIAI

SEQ ID NO: 33 - protein, 17 aa, synthetic construct
  REGION 1..17: note = Synthetic Sequence
LPPLLTDEMIAQYTSAL

SEQ ID NO: 34 - protein, 10 aa, synthetic construct
  REGION 1..10: note = Synthetic Sequence
MIAQYTSALL

SEQ ID NO: 35 - protein, 18 aa, synthetic construct
  REGION 1..18: note = Synthetic Sequence
LPPLLTDEMIAQYTSALL

SEQ ID NO: 36 - protein, 177 aa, synthetic construct
  REGION 1..177: note = Synthetic Sequence
PAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLK

SEQ ID NO: 37 - protein, 304 aa, synthetic construct
  REGION 1..304: note = Synthetic Sequence
ASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLK

SEQ ID NO: 38 - protein, 304 aa, synthetic construct
  REGION 1..304: note = Synthetic Sequence
ASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLK

SEQ ID NO: 39 - protein, 330 aa, synthetic construct
  REGION 1..330: note = Synthetic Sequence
MFVFLVLLPLVSSQCVNLTTRTQLPPASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLK

SEQ ID NO: 40 - protein, 471 aa, synthetic construct
  REGION 1..471: note = Synthetic Sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLK

SEQ ID NO: 41 - protein, 406 aa, synthetic construct
  REGION 1..406: note = Synthetic Sequence
MFVFLVLLPLVSSQCVNLTTRTQLPPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLK

SEQ ID NO: 42 - protein, 26 aa, synthetic construct
  REGION 1..26: note = Synthetic Sequence
MFVFLVLLPLVSSQCVNLTTRTQLPP

SEQ ID NO: 43 - DNA, 1565 bp, synthetic construct
  misc_feature 1..1565: note = Synthetic Sequence
  source 1..1565: mol_type = other DNA
agcaaaagcagggtagataatcactcactgagtgacatcaaaatcatggcgtctcaaggcaccaaacgatcttacgaacagatggagactgatggagaacgccagaatgccactgaaatcagagcatccgtcggaaaaatgattggtggaattggacgattctacatccaaatgtgcaccgaactcaaactcagtgattatgagggacggttgatccaaaacagcttaacaatagagagaatggtgctctctgcttttgacgaaaggagaaataaataccttgaagaacatcccagtgcggggaaagatcctaagaaaactggaggacctatatacaggagagtaaacggaaagtggatgagagaactcatcctttatgacaaagaagaattaaggcgaatctggcgccaagctaataatggtgacgatgcaacggctggtctgactcacatgatgatctggcattccaatttgaatgatgcaacttatcagaggacaagagctcttgttcgcaccggaatggatcccaggatgtgctctctgatgcaaggttcaactctccctaggaggtctggagccgcaggtgctgcagtcaaaggagttggaacaatggtgatggaattggtcagaatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccggaacccagggaatgctgagttcgaagatctcacttttctagcacggtctgcactcatattgagagggtcggttgctcacaagtcctgcctgcctgcctgtgtgtatggacctgccgtagccagtgggtacgactttgaaaggaagggatactctctagtcggaatagaccctttcagactgcttcaaaacagccaagtgtacagcctaatcagaccaaatgagaatccagcacacaagagtcaactggtgtggatggcatgccattctgccgcatttgaagatctaagagtattaagcttcatcaaagggacgaaggtgctcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccattatggcagcattcaatgggaatacagaggggagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 44 - DNA, 2341 bp, synthetic construct
  misc_feature 1..2341: note = Synthetic Sequence
  source 1..2341: mol_type = other DNA
agcaaaagcaggcaaaccatttgaatggatgtcaatccgaccttacttttcttaaaagtgccagcacaaaatgctataagcacaactttcccttatactggagatcctccttacagccatgggacaggaacaggatacaccttggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaggcgatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggatgtaatggagtcaatgaacaaagaagaaatgtggatcacaactcattttcagagaaagagacgggtgagagacaatatgactaagaaaatgataacacagagaacaatgggtaaaaagaagcagagattgaacaaaaggagttatctaattagagcattgaccctgaacacaatgaccaaagatgctgagagagggaagctaaaacggagagcaattgcaaccccagggatgcaaataagggggtttgtatactttgttgagacactggcaaggagtatatgtgagaaacttgaacaatcagggttgccagttggaggcaatgagaagaaagcaaagttggcaaatgttgtaaggaagatgatgaccaattctcaggacaccgaactttctttcaccatcactggagataacaccaaatggaacgaaaatcagaatcctcggatgtttttggccatgatcacatatatgaccagaaatcagcccgaatggttcagaaatgttctaagtattgctccaataatgttctcaaacaaaatggcgagactgggaaaagggtatatgtttgagagcaagagtatgaaacttagaactcaaatacctgcagaaatgctagcaagcatcgatttgaaatatttcaatgattcaacaagaaagaagattgaaaaaatccgaccgctcttaatagaggggactgcatcattgagccctggaatgatgatgggcatgttcaatatgttaagcactgtattaggcgtctccatcctgaatcttggacaaaagagatacaccaagactacttactggtgggatggtcttcaatcctctgacgattttgctctgattgtgaatgcacccaatcatgaagggattcaagccggagtcaacaggttttatcgaacctgtaagctacttggaatcaatatgagcaagaaaaagtcttacataaacagaacaggtacatttgaattcacaagttttttctatcgttatgggtttgttgccaatttcagcatggagcttcccagttttggggtgtctgggatcaacgagtcagcggacatgagtattggagttactgtcatcaaaaacaatatgataaacaatgatcttggtccagcaacagctcaaatggcccttcagttgttcatcaaagattacaggtacacgtaccgatgccatataggtgacacacaaatacaaacccgaagatcatttgaaataaagaaactgtgggagcaaacccgttccaaagctggactgctagtctccgacggaggcccaaatttatacaacattagaaatctccacattcctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaggatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 45 - DNA, 1565 bp, synthetic construct
  misc_feature 1..1565: note = Synthetic Sequence
  source 1..1565: mol_type = other DNA
agcaaaagcagggtagataatcactcactgagtgacatcaaaatcatggcgtctcaaggcaccaaacgatcttacgaacagatggagactgatggagaacgccagaatgccactgaaatcagagcatccgtcggaaaaatgattggtggaattggacgattctacatccaaatgtgcaccgaactcaaactcagtgattatgagggacggttgatccaaaacagcttaacaatagagagaatggtgctctctgcttttgacgaaaggagaaataaataccttgaagaacatcccagtgcggggaaagatcctaagaaaactggaggacctatatacaggagagtaaacggaaagtggatgagagaactcatcctttatgacaaagaagaattaaggcgaatctggcgccaagctaataatggtgacgatgcaacggctggtctgactcacatgatgatctggcattccaatttgaatgatgcaacttatcagaggacaagagctcttgttcgcaccggaatggatcccaggatgtgctctctgatgcaaggttcaactctccctaggaggtctggagccgcaggtgctgcagtcaaaggagttggaacaatggtgatggaattggtcagaatgatcaaacgtgggatcaatgatcggaacttctggaggggtgagaatggacgaaaaacaagaattgcttatgaaagaatgtgcaacattctcaaagggaaatttcaaactgctgcacaaaaagcaatgatggatcaagtgagagagagccggaacccagggaatgctgagttcgaagatctcacttttctagcacggtctgcactcatattgagagggtcggttgctcacaagtcctgcctgcctgcctgtgtgtatggacctgccgtagccagtgggtacgactttgaaagggagggatactctctagtcggaatagaccctttcagactgcttcaaaacagccgagtgtacagcctaatcagaccaaatgagaatccagcacacaagagtcaactggtgtggatggcatgccattctgccgcatttgaagatctaagagtattaagcttcatcaaagggacgaaggtgctcccaagagggaagctttccactagaggagttcaaattgcttccaatgaaaatatggagactatggaatcaagtacacttgaactgagaagcaggtactgggccataaggaccagaagtggaggaaacaccaatcaacagagggcatctgcgggccaaatcagcatacaacctacgttctcagtacagagaaatctcccttttgacagaacaaccattatggcagcattcaatgggaatacagaggggagaacatctgacatgaggaccgaaatcataaggatgatggaaagtgcaagaccagaagatgtgtctttccaggggcggggagtcttcgagctctcggacgaaaaggcagcgagcccgatcgtgccttcctttgacatgagtaatgaaggatcttatttcttcggagacaatgcagaggagtacgacaattaaagaaaaatacccttgtttctact

SEQ ID NO: 46 - DNA, 2341 bp, synthetic construct
  misc_feature 1..2341: note = Synthetic Sequence
  source 1..2341: mol_type = other DNA
agcaaaagcaggcaaaccatttgaatggatgtcaatccgaccttacttttcttaaaagtgccagcacaaaatgctataagcacaactttcccttatactggagatcctccttacagccatgggacaggaacaggatacaccttggatactgtcaacaggacacatcagtactcagaaaagggaagatggacaacaaacaccgaaactggagcaccgcaactcaacccgattgatgggccactgccagaagacaatgaaccaagtggttatgcccaaacagattgtgtattggaggcgatggctttccttgaggaatcccatcctggtatttttgaaaactcgtgtattgaaacgatggaggttgttcagcaaacacgagtagacaagctgacacaaggccgacagacctatgactggactctaaatagaaaccaacctgctgcaacagcattggccaacacaatagaagtgttcagatcaaatggcctcacggccaatgagtctggaaggctcatagacttccttaaggatgtaatggagtcaatgaacaaagaagaaatgtggatcacaactcattttcagagaaagagacgggtgagagacaatatgactaagaaaatgataacacagagaacaatgggtaaaaagaagcagagattgaacaaaaggagttatctaattagagcattgaccctgaacacaatgaccaaagatgctgagagagggaagctaaaacggagagcaattgcaaccccagggatgcaaataagggggtttgtatactttgttgagacactggcaaggagtatatgtgagaaacttgaacaatcagggttgccagttggaggcaatgagaagaaagcaaagttggcaaatgttgtaaggaagatgatgaccaattctcaggacaccgaactttctttcaccatcactggagataacaccaaatggaacgaaaatcagaatcctcggatgtttttggccatgatcacatatatgaccagaaatcagcccgaatggttcagaaatgttctaagtattgctccaataatgttctcaaacaaaatggcgagactgggaaaagggtatatgtttgagagcaagagtatgaaacttagaactcaaatacctgcagaaatgctagcaagcatcgatttgaaatatttcaatgattcaacaagaaagaagattgaaaaaatccgaccgctcttaatagaggggactgcatcattgagccctggaatgatgatgggcatgttcaatatgttaagcactgtattaggcgtctccatcctgaatcttggacaaaagagatacaccaagactacttactggtgggatggtcttcaatcctctgacgattttgctctgattgtgaatgcacccaatcatgaagggattcaagccggagtcgacaggttttatcgaacctgtaagctacttggaatcaatatgagcaagaaaaagtcttacataaacagaacaggtacatttgaattcacaagttttttctatcgttatgggtttgttgccaatttcagcatggagcttcccagttttggggtgtctgggatcaacgagtcagcggacatgagtattggagttactgtcatcaaaaacaatatgataaacaatgatcttggtccagcaacagctcaaatggcccttcagttgttcatcaaagattacaggtacacgtaccgatgccatataggtgacacacaaatacaaacccgaagatcatttgaaataaagaaactgtgggagcaaacccgttccaaagctggactgctagtctccgacggaggcccaaatttatacaacattagaaatctccacatttctgaagtctgcctaaaatgggaattgatggatgaggattaccaggggcgtttatgcaacccactgaacccatttgtcagccataaagaaattgaatcaatgaacaatgcagtgatgatgccagcacatggtccagccaaaaacatggagtatgatgctgttgcaacaacacactcctggatccccaaaagaaatcgatccatcttgaatacaagtcaaagaggagtacttgaggatgaacaaatgtaccaaaggtgctgcaatttatttgaaaaattcttccccagcagttcatacagaagaccagtcgggatatccagtatggtggaggctatggtttccagagcccgaattgatgcacggattgatttcgaatctggaaggataaagaaagaagagttcactgagatcatgaagatctgttccaccattgaagagctcagacggcaaaaatagtgaatttagcttgtccttcatgaaaaaatgccttgtttctact

SEQ ID NO: 47 - DNA, 2341 bp, synthetic construct
  misc_feature 1..2341: note = Synthetic Sequence
  source 1..2341: mol_type = other DNA
agcaaaagcaggtcaattatattcaatatggaaagaataaaagaactacgaaatctaatgtcgcagtctcgcacccgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcaaggacaaactttatggagtaaaatgaatgatgccggatcagaccgagtgatggtatcacctctggctgtgacatggtggaataggaatggaccaataacaaatacagttcattatccaaaaatctacaaaacttattttgaaagagtcgaaaggctaaagcatggaacctttggccctgtccattttagaaaccaagtcaaaatacgtcggagagttgacataaatcctggtcatgcagatctcagtgccaaggaggcacaggatgtaatcatggaagttgttttccctaacgaagtgggagccaggatactaacatcggaatcgcaactaacgataaccaaagagaagaaagaagaactccaggattgcaaaatttctcctttgatggttgcatacatgttggagagagaactggtccgcaaaacgagattcctcccagtggctggtggaacaagcagtgtgtacattgaagtgttgcatttgactcaaggaacatgctgggaacagatgtatactccaggaggggaagtgaggaatgatgatgttgatcaaagcttgattattgctgctaggaacatagtgagaagagctgcagtatcagcagatccactagcatctttattggagatgtgccacagcacacagattggtggaattaggatggtagacatccttaggcagaacccaacagaagagcaagccgtggatatatgcaaggctgcaatgggactgagaattagctcatccttcagttttggtggattcacatttaagagaacaagcggatcatcagtcaagagagaggaagaggtgcttacgggcaatcttcaaacattgaagataagagtgcatgagggatatgaagagttcacaatggttgggagaagagcaacagccatactcagaaaagcaaccaggagattgattcagctgatagtgagtgggagagacgaacagtcgattgccgaagcaataattgtggccatggtattttcacaagaggattgtatgataaaagcagtcagaggtgatctgaatttcgtcaatagggcgaatcaacgattgaatcctatgcatcaacttttaagacattttcagaaggatgcgaaagtgctttttcaaaattggggagttgaacctatcgacaatgtgatgggaatgattgggatattgcccgacataactccaagcatcgagatgtcaatgagaggagtgagaatcagcaaaatgggtgtagatgagtactccagcacggagagggtagtggtgagcattgaccgttttttgagagtccgggaccaacgaggaaatgtactactgtctcccgaggaggtcagtgaaacacagggaacagagaaactgacagtaacttactcatcgtcaatgatgtgggagattaatggtcctgaatcagtgttggtcaatacctatcaatggatcatcagaaactgggaaactgttaaaattcagtggtcccagaaccctacaatgctatacaataaaatggaatttgaaccatttcagtctttagtacctaaggccattagaggccaatacagtgggtttgtaagaactctgttccaacaaatgagggatgtgcttgggacatttgataccgcacagataataaaacttcttcccttcgcagccgctccaccaaagcaaagtagaatgcagttctcctcatttactgtgaatgtgaggggatcaggaatgagaatacttgtaaggggcaattctcctgtattcaactataacaaggccacgaagagactcacagttctcggaaaggatgctggcactttaactgaagacccagatgaaggcacagctggagtggagtccgctgttctgaggggattcctcattctgggcaaagaagacaagagatatgggccagcactaagcatcaatgaactgagcaaccttgcgaaaggagagaaggctaatgtgctaattgggcaaggagacgtggtgttggtaatgaaacggaaacgggactctagcatacttactgacagccagacagcgaccaaaagaattcggatggccatcaattagtgtcgaatagtttaaaaacgaccttgtttctact

SEQ ID NO: 48 - protein, 498 aa, synthetic construct
  REGION 1..498: note = Synthetic Sequence
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEELRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFERKGYSLVGIDPFRLLQNSQVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDN

SEQ ID NO: 49 - protein, 757 aa, synthetic construct
  REGION 1..757: note = Synthetic Sequence
MDVNPTLLFLKVPAQNAISTTFPYTGDPPYSHGTGTGYTLDTVNRTHQYSEKGRWTTNTETGAPQLNPIDGPLPEDNEPSGYAQTDCVLEAMAFLEESHPGIFENSCIETMEVVQQTRVDKLTQGRQTYDWTLNRNQPAATALANTIEVFRSNGLTANESGRLIDFLKDVMESMNKEEMWITTHFQRKRRVRDNMTKKMITQRTMGKKKQRLNKRSYLIRALTLNTMTKDAERGKLKRRAIATPGMQIRGFVYFVETLARSICEKLEQSGLPVGGNEKKAKLANVVRKMMTNSQDTELSFTITGDNTKWNENQNPRMFLAMITYMTRNQPEWFRNVLSIAPIMFSNKMARLGKGYMFESKSMKLRTQIPAEMLASIDLKYFNDSTRKKIEKIRPLLIEGTASLSPGMMMGMFNMLSTVLGVSILNLGQKRYTKTTYWWDGLQSSDDFALIVNAPNHEGIQAGVNRFYRTCKLLGINMSKKKSYINRTGTFEFTSFFYRYGFVANFSMELPSFGVSGINESADMSIGVTVIKNNMINNDLGPATAQMALQLFIKDYRYTYRCHIGDTQIQTRRSFEIKKLWEQTRSKAGLLVSDGGPNLYNIRNLHIPEVCLKWELMDEDYQGRLCNPLNPFVSHKEIESMNNAVMMPAHGPAKNMEYDAVATTHSWIPKRNRSILNTSQRGVLEDEQMYQRCCNLFEKFFPSSSYRRPVGISSMVEAMVSRARIDARIDFESGRIKKEEFTEIMKICSTIEELRRQK

SEQ ID NO: 50 - protein, 498 aa, synthetic construct
  REGION 1..498: note = Synthetic Sequence
MASQGTKRSYEQMETDGERQNATEIRASVGKMIGGIGRFYIQMCTELKLSDYEGRLIQNSLTIERMVLSAFDERRNKYLEEHPSAGKDPKKTGGPIYRRVNGKWMRELILYDKEELRRIWRQANNGDDATAGLTHMMIWHSNLNDATYQRTRALVRTGMDPRMCSLMQGSTLPRRSGAAGAAVKGVGTMVMELVRMIKRGINDRNFWRGENGRKTRIAYERMCNILKGKFQTAAQKAMMDQVRESRNPGNAEFEDLTFLARSALILRGSVAHKSCLPACVYGPAVASGYDFEREGYSLVGIDPFRLLQNSRVYSLIRPNENPAHKSQLVWMACHSAAFEDLRVLSFIKGTKVLPRGKLSTRGVQIASNENMETMESSTLELRSRYWAIRTRSGGNTNQQRASAGQISIQPTFSVQRNLPFDRTTIMAAFNGNTEGRTSDMRTEIIRMMESARPEDVSFQGRGVFELSDEKAASPIVPSFDMSNEGSYFFGDNAEEYDN

SEQ ID NO: 51 - protein, 757 aa, synthetic construct
  REGION 1..757: note = Synthetic Sequence
MDVNPTLLFLKVPAQNAISTTFPYTGDPPYSHGTGTGYTLDTVNRTHQYSEKGRWTTNTETGAPQLNPIDGPLPEDNEPSGYAQTDCVLEAMAFLEESHPGIFENSCIETMEVVQQTRVDKLTQGRQTYDWTLNRNQPAATALANTIEVFRSNGLTANESGRLIDFLKDVMESMNKEEMWITTHFQRKRRVRDNMTKKMITQRTMGKKKQRLNKRSYLIRALTLNTMTKDAERGKLKRRAIATPGMQIRGFVYFVETLARSICEKLEQSGLPVGGNEKKAKLANVVRKMMTNSQDTELSFTITGDNTKWNENQNPRMFLAMITYMTRNQPEWFRNVLSIAPIMFSNKMARLGKGYMFESKSMKLRTQIPAEMLASIDLKYFNDSTRKKIEKIRPLLIEGTASLSPGMMMGMFNMLSTVLGVSILNLGQKRYTKTTYWWDGLQSSDDFALIVNAPNHEGIQAGVDRFYRTCKLLGINMSKKKSYINRTGTFEFTSFFYRYGFVANFSMELPSFGVSGINESADMSIGVTVIKNNMINNDLGPATAQMALQLFIKDYRYTYRCHIGDTQIQTRRSFEIKKLWEQTRSKAGLLVSDGGPNLYNIRNLHISEVCLKWELMDEDYQGRLCNPLNPFVSHKEIESMNNAVMMPAHGPAKNMEYDAVATTHSWIPKRNRSILNTSQRGVLEDEQMYQRCCNLFEKFFPSSSYRRPVGISSMVEAMVSRARIDARIDFESGRIKKEEFTEIMKICSTIEELRRQK

SEQ ID NO: 52 - protein, 759 aa, synthetic construct
  REGION 1..759: note = Synthetic Sequence
MERIKELRNLMSQSRTREILTKTTVDHMAIIKKYTSGRQEKNPALRMKWMMAMKYPITADKRITEMIPERNEQGQTLWSKMNDAGSDRVMVSPLAVTWWNRNGPITNTVHYPKIYKTYFERVERLKHGTFGPVHFRNQVKIRRRVDINPGHADLSAKEAQDVIMEVVFPNEVGARILTSESQLTITKEKKEELQDCKISPLMVAYMLERELVRKTRFLPVAGGTSSVYIEVLHLTQGTCWEQMYTPGGEVRNDDVDQSLIIAARNIVRRAAVSADPLASLLEMCHSTQIGGIRMVDILRQNPTEEQAVDICKAAMGLRISSSFSFGGFTFKRTSGSSVKREEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDAKVLFQNWGVEPIDNVMGMIGILPDITPSIEMSMRGVRISKMGVDEYSSTERVVVSIDRFLRVRDQRGNVLLSPEEVSETQGTEKLTVTYSSSMMWEINGPESVLVNTYQWIIRNWETVKIQWSQNPTMLYNKMEFEPFQSLVPKAIRGQYSGFVRTLFQQMRDVLGTFDTAQIIKLLPFAAAPPKQSRMQFSSFTVNVRGSGMRILVRGNSPVFNYNKATKRLTVLGKDAGTLTEDPDEGTAGVESAVLRGFLILGKEDKRYGPALSINELSNLAKGEKANVLIGQGDVVLVMKRKRDSSILTDSQTATKRIRMAIN

SEQ ID NO: 53 - DNA, 976 bp, synthetic construct
  misc_feature 1..976: note = Synthetic Sequence
  source 1..976: mol_type = other DNA
agcaaaagcaggtagatattgaaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgattaataggatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctact

SEQ ID NO: 54 - protein, 24 aa, synthetic construct
  REGION 1..24: note = Synthetic Sequence
MSLLTEVETPIRNEWGCRCNGSSD

SEQ ID NO: 55 - DNA, 1069 bp, synthetic construct
  misc_feature 1..1069: note = Synthetic Sequence
  source 1..1069: mol_type = other DNA
agcaaaagcaggtagatattgaaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagctaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgatccttattctgtcctatataattccgcatcattttccacttttaagtgttatggagtggcaggtgcaggtgattacaaggatgacgacgataagtaataggatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctact

SEQ ID NO: 56 - DNA, 2341 bp, synthetic construct
  misc_feature 1..2341: note = Synthetic Sequence
  source 1..2341: mol_type = other DNA
agcaaaagcaggtcaattatattcaatatggaaagaataaaagaactacgaaatctaatgtcgcagtctcgcacccgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcaaggacaaactttatggagtaaaatgaatgatgccggatcagaccgagtgatggtatcacctctggctgtgacatggtggaataggaatggaccaataacaaatacagttcattatccaaaaatctacaaaacttattttgaaagagtcgaaaggctaaagcatggaacctttggccctgtccattttagaaaccaagtcaaaatacgtcggagagttgacataaatcctggtcatgcagatctcagtgccaaggaggcacaggatgtaatcatggaagttgttttccctaacgaagtgggagccaggatactaacatcggaatcgcaactaacgataaccaaagagaagaaagaagaactccaggattgcaaaatttctcctttgatggttgcatacatgttggagagagaactggtccgcaaaacgagattcctcccagtggctggtggaacaagcagtgtgtacattgaagtgttgcatttgactcaaggaacatgctgggaacagatgtatactccaggaggggaagtgaggaatgatgatgttgatcaaagcttgattattgctgctaggaacatagtgagaagagctgcagtatcagcagatccactagcatctttattggagatgtgccacagcacacagattggtggaattaggatggtagacatccttaggcagaacccaacagaagagcaagccgtggatatatgcaaggctgcaatgggactgagaattagctcatccttcagttttggtggattcacatttaagagaacaagcggatcatcagtcaagagagaggaagaggtgcttacgggcaatcttcaaacattgaagataagagtgcatgagggatatgaagagttcacaatggttgggagaagagcaacagccatactcagaaaagcaaccaggagattgattcagctgatagtgagtgggagagacgaacagtcgattgccgaagcaataattgtggccatggtattttcacaagaggattgtatgataaaagcagtcagaggtgatctgaatttcgtcaatagggcgaatcaacgattgaatcctatgcatcaacttttaagacattttcagaaggatgcgaaagtgctttttcaaaattggggagttgaacctatcgacaatgtgatgggaatgattgggatattgcccgacatgactccaagcatcgagatgtcaatgagaggagtgagaatcagcaaaatgggtgtagatgagtactccagcacggagagggtagtggtgagcattgaccgttttttgagagtccgggaccaacgaggaaatgtactactgtctcccgaggaggtcagtgaaacacagggaacagagaaactgacaataacttactcatcgtcaatgatgtgggagattaatggtcctgaatcagtgttggtcaatacctatcaatggatcatcagaaactgggaaactgttaaaattcagtggtcccagaaccctacaatgctatacaataaaatggaatttgaaccatttcagtctttagtacctaaggccattagaggccaatacagtgggtttgtaagaactctgttccaacaaatgagggatgtgcttgggacatttgataccgcacagataataaaacttcttcccttcgcagccgctccaccaaagcaaagtagaatgcagttctcctcatttactgtgaatgtgaggggatcaggaatgagaatacttgtaaggggcaattctcctgtattcaactataacaaggccacgaagagactcacagttctcggaaaggatgctggcactttaactgaagacccagatgaaggcacagctggagtggagtccgctgttctgaggggattcctcattctgggcaaagaagacaagagatatgggccagcactaagcatcaatgaactgagcaaccttgcgaaaggagagaaggctaatgtgctaattgggcaaggagacgtggtgttggtaatgaaacggaaacgggactctagcatacttactgacagccagacagcgaccaaaagaattcggatggccatcaattagtgtcgaatagtttaaaaacgaccttgtttctact

SEQ ID NO: 57 - protein, 759 aa, synthetic construct
  REGION 1..759: note = Synthetic Sequence
MERIKELRNLMSQSRTREILTKTTVDHMAIIKKYTSGRQEKNPALRMKWMMAMKYPITADKRITEMIPERNEQGQTLWSKMNDAGSDRVMVSPLAVTWWNRNGPITNTVHYPKIYKTYFERVERLKHGTFGPVHFRNQVKIRRRVDINPGHADLSAKEAQDVIMEVVFPNEVGARILTSESQLTITKEKKEELQDCKISPLMVAYMLERELVRKTRFLPVAGGTSSVYIEVLHLTQGTCWEQMYTPGGEVRNDDVDQSLIIAARNIVRRAAVSADPLASLLEMCHSTQIGGIRMVDILRQNPTEEQAVDICKAAMGLRISSSFSFGGFTFKRTSGSSVKREEEVLTGNLQTLKIRVHEGYEEFTMVGRRATAILRKATRRLIQLIVSGRDEQSIAEAIIVAMVFSQEDCMIKAVRGDLNFVNRANQRLNPMHQLLRHFQKDAKVLFQNWGVEPIDNVMGMIGILPDMTPSIEMSMRGVRISKMGVDEYSSTERVVVSIDRFLRVRDQRGNVLLSPEEVSETQGTEKLTITYSSSMMWEINGPESVLVNTYQWIIRNWETVKIQWSQNPTMLYNKMEFEPFQSLVPKAIRGQYSGFVRTLFQQMRDVLGTFDTAQIIKLLPFAAAPPKQSRMQFSSFTVNVRGSGMRILVRGNSPVFNYNKATKRLTVLGKDAGTLTEDPDEGTAGVESAVLRGFLILGKEDKRYGPALSINELSNLAKGEKANVLIGQGDVVLVMKRKRDSSILTDSQTATKRIRMAIN

SEQ ID NO: 58 - DNA, 2233 bp, synthetic construct
  misc_feature 1..2233: note = Synthetic Sequence
  source 1..2233: mol_type = other DNA
agcaaaagcaggtactgatccaaaatggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagattttcacttcatcaatgagcaaggcgagtcaataatcgtagaacttggtgatccaaatgcacttttgaagcacagatttgaaataatcgagggaagagatcgcacaatggcctggacagtagtaaacagtatttgcaacactacaggggctgagaaaccaaagtttctaccagatttgtatgattacaaggagaatagattcatcgaaattggagtaacaaggagagaagttcacatatactatctggaaaaggccaataaaattaaatctgagaaaacacacatccacattttctcgttcactggggaagaaatggccacaaaggcagactacactctcgatgaagaaagcagggctaggatcaaaaccagactattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaagaaaacaagtcagctaaagtgggcacttggtgagaacatggcaccagaaaaggtagactttgacgactgtaaagatgtaggtgatttgaagcaatatgatagtgatgaaccagaattgaagtcgcttgcaagttggattcagaatgagtttaacaaggcatgcgaactgacagattcaagctggatagagctcgatgagattggagaagatgtggctccaattgaacacattgcaagcatgagaaggaattatttcacatcagaggtgtctcactgcagagccacagaatacataatgaagggagtgtacatcaatactgccttgcttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacataaatgggagaagtactgtgttcttgagataggagatatgcttataagaagtgccataggccaggtttcaaggcccatgttcttgtatgtgagaacaaatggaacctcaaaaattaaaatgaaatggggaatggagatgaggcgttgcctcctccagtcacttcaacaaattgagagtatgattgaagctgagtcctctgtcaaagagaaagacatgaccaaagagttctttgagaacaaatcagaaacatggcccattggagagtcccccaaaggagtggaggaaagttccattgggaaggtctgcaggactttattagcaaagtcggtattcaacagcttgtatgcatctccacaactagaaggattttcagctgaatcaagaaaactgcttcttatcgttcaggctcttagggacaacctggaacctgggacctttgatcttggggggctatatgaagcaattgaggagtgcctgattaatgatccctgggttttgcttaatgcttcttggttcaactccttccttacacatgcattgagttagttgtggcagtgctactatttgctatccatactgtccaaaaaagtaccttgtttctact

SEQ ID NO: 59 - protein, 716 aa, synthetic construct
  REGION 1..716: note = Synthetic Sequence
MEDFVRQCFNPMIVELAEKTMKEYGEDLKIETNKFAAICTHLEVCFMYSDFHFINEQGESIIVELGDPNALLKHRFEIIEGRDRTMAWTVVNSICNTTGAEKPKFLPDLYDYKENRFIEIGVTRREVHIYYLEKANKIKSEKTHIHIFSFTGEEMATKADYTLDEESRARIKTRLFTIRQEMASRGLWDSFRQSERGEETIEERFEITGTMRKLADQSLPPNFSSLENFRAYVDGFEPNGYIEGKLSQMSKEVNARIEPFLKTTPRPLRLPNGPPCSQRSKFLLMDALKLSIEDPSHEGEGIPLYDAIKCMRTFFGWKEPNVVKPHEKGINPNYLLSWKQVLAELQDIENEEKIPKTKNMKKTSQLKWALGENMAPEKVDFDDCKDVGDLKQYDSDEPELKSLASWIQNEFNKACELTDSSWIELDEIGEDVAPIEHIASMRRNYFTSEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLIRSAIGQVSRPMFLYVRTNGTSKIKMKWGMEMRRCLLQSLQQIESMIEAESSVKEKDMTKEFFENKSETWPIGESPKGVEESSIGKVCRTLLAKSVFNSLYASPQLEGFSAESRKLLLIVQALRDNLEPGTFDLGGLYEAIEECLINDPWVLLNASWFNSFLTHALS

SEQ ID NO: 60 - DNA, 890 bp, synthetic construct
  misc_feature 1..890: note = Synthetic Sequence
  source 1..890: mol_type = other DNA
agcaaaagcagggtgacaaaaacataatggatccaaacactgtgtcaagctttcaggtagattgctttctttggcatgtccgcaaacgagttgcagaccaagaactaggcgatcccccattccttgatcggcttcgccgagatcagaaatccctaagaggaaggggcagtactctcggtctggacatcaagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcaaaatggaccaggcgatcatggataagaacatcatactgaaagcgaacttcagtgtgatttttgaccggctggagactctaatattgctaagggctttcaccgaagagggagcaattgttggcgaaatttcaccattgccttctcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttagtactaaaaaacacccttgtttctact

SEQ ID NO: 61 - protein, 230 aa, synthetic construct
  REGION 1..230: note = Synthetic Sequence
MDPNTVSSFQVDCFLWHVRKRVADQELGDPPFLDRLRRDQKSLRGRGSTLGLDIKTATRAGKQIVERILKEESDEALKMTMASVPASRYLTDMTLEEMSRDWSMLIPKQKVAGPLCIKMDQAIMDKNIILKANFSVIFDRLETLILLRAFTEEGAIVGEISPLPSLPGHTAEDVKNAVGVLIGGLEWNDNTVRVSETLQRFAWRSSNENGRPPLTPKQKREMAGTIRSEV

SEQ ID NO: 62 - protein, 121 aa, synthetic construct
  REGION 1..121: note = Synthetic Sequence
MDPNTVSSFQDILLRMSKMQLESSSEDLNGMITQFESLKLYRDSLGEAVMRMGDLHSLQNRNEKWREQLGQKFEEIRWLIEEVRHKLKITENSFEQITFMQALHLLLEVEQEIRTFSFQLI

SEQ ID NO: 63 - DNA, 2369 bp, synthetic construct
  misc_feature 1..2369: note = Synthetic Sequence
  source 1..2369: mol_type = other DNA
agcagaagcggagcctttaagatgaatataaatccttattttctcttcatagatgtacccatacaggcagcaatttcaacaacattcccatacaccggtgttcccccttattcccatggaacgggaacaggctacacaatagacaccgtgatcagaacacatgagtactcaaacaagggaaaacagtacatttctgatgttacaggatgtacaatggtagatccaacaaatgggccattacccgaagacaatgagccgagtgcctatgcacaattagattgcgttctggaggctttggatagaatggatgaagaacatccaggtctgtttcaagcagcctcacagaatgccatggaggcactaatggtcacaactgtagacaaattaacccaggggagacagacttttgattggacagtatgcagaaaccaacctgctgcaacggcactgaacacaacaataacctcttttaggttgaatgatttgaatggagccgacaagggtggattagtacccttttgccaagatatcattgattcattggacagacctgaaatgactttcttctcagtaaagaatataaagaaaaaattgcctgctaaaaacagaaagggtttcctcataaagagaataccaatgaaggtaaaagacagaataaccagagtggaatacatcaaaagagcattatcattaaacacaatgacaaaagatgctgaaagaggcaaactaaaaagaagagcgattgccaccgctggaatacaaatcagagggtttgtattagtagttgaaaacttggctaaaaatatctgtgaaaatctagaacaaagtggtttgccagtaggtggaaacgagaagaaggccaaactgtcaaatgcagtggccaaaatgctcagtaactgcccaccaggagggatcagcatgacagtaacaggagacaataccaaatggaatgaatgcttaaatccaagaatctttttggctatgactgaaagaataaccagagacagcccaatttggttccgggatttttgtagtatagcaccggtcttgttctccaataaaatagccagattgggaaaagggtttatgataacaagcaaaacaaaaagactgaaggctcaaataccttgtcctgatctgtttagtataccattagaaagatataatgaagaaacaagggcaaaattgaaaaagctgaaaccattcttcaatgaagaaggaacggcatctttgtcgcctgggatgatgatgggaatgtttaatatgctatctaccgtgttgggagtagccgcactaggtatcaaaaacattggaaacaaagaatacttatgggatggactgcaatcttctgatgattttgctctgtttgttaatgcaaaagatgaagagacatgtatggaaggaataaacgacttttaccgaacatgtaaactattgggaataaacatgagcaaaaagaaaagttactgtaatgaaactggaatgtttgaatttacaagcatgttctacagagatggatttgtatctaattttgcaatggaacttccttcatttggagttgctggagtaaatgaatcagcagatatggcaataggaatgacaataataaagaacaatatgatcaacaatgggatgggtccagcaacagcacaaacagccatacaattattcatagctgattatagatacacctacaaatgccacaggggagattccaaagtggaaggaaagagaatgaaaattataaaggagctatgggaaaacactaaaggaagagatggtctgttagtagcagatggtgggcctaacatttacaatttgagaaacttgcatatcccagaaatagtattaaagtacaacctaatggaccctgaatacaaagggcggttacttcatcctcaaaatccctttgtaggacatttgtctattgagggcatcaaagaggcagatataaccccagcacatggtccagtaaagaaaatggactatgatgcggtgtctggaactcatagttggagaaccaaaaggaacagatctatactaaacactgatcagaggaacatgattcttgaggaacaatgctacgctaagtgttgcaacctttttgaggcctgttttaacagtgcatcatacaggaaaccagtaggtcagcacagcatgcttgaggctatggcccacagattaagaatggatgcacgactagattatgaatcaggaagaatgtcaaaggatgattttgagaaagcaatggctcaccttggtgagattgggtacatataagcttcgaagatgtctatggggttattggtcatcattgaatacatgcggtacacaaatgattaaaatgaaaaaaggctcgtgtttctact

SEQ ID NO: 64 - DNA, 2396 bp, synthetic construct
  misc_feature 1..2396: note = Synthetic Sequence
  source 1..2396: mol_type = other DNA
agcagaagcggagcgttttcaagatgacattggctaaaattgaattgttaaaacaactgttaagggacaacgaagccaaaacagtattgaaacaaacaacggtagaccaatataacataataagaaaattcaatacatcaagaattgaaaagaacccttcattaaggatgaagtgggccatgtgttctaattttcccttggctctgaccaagggtgatatggcaaatagaatccccttggaatacaagggaatacaacttaaaacaaatgctgaagacataggaaccaaaggccaaatgtgctcaatagcagcagttacctggtggaatacatatggaccaataggagacactgaaggtttcgaaaaggtctacgaaagcttctttctcagaaagatgagacttgacaatgccacttggggccgaataacttttggcccagttgaaagagtaagaaaaagggtactgctaaaccctctcaccaaggaaatgcctccagatgaagcgagcaatgtgataatggaaatattgttccctaaggaagcaggaataccaagagaatctacttggatacatagggaactgataaaagaaaaaagagaaaaattgaaaggaacgatgataactcccattgtactggcatacatgcttgagagagaattggttgcccgaagaaggttcctgccagtggcaggagcaacatcagctgagttcatagaaatgctacactgcttacaaggtgaaaattggagacaaatatatcacccaggagggaataaactaactgaatctaggtctcaatcaatgattgtagcttgtagaaaaataatcagaagatcaatagtcgcatcaaacccactagagctagctgtagaaattgcaaacaagactgtgatagatactgaacctttaaaatcatgtctggcagccatagacggaggtgatgtagcctgtgacataatgagagctgcattaggactaaagatcagacaaagacaaagatttggacggcttgaactaaagagaatatcaggaagaggattcaaaaatgatgaagaaatattgatcgggaacggaacaatacagaagattggaatatgggacggagaagaggagttccatgtaagatgtggtgaatgcaggggaatattaaaaaagagcaaaatgagaatggaaaaactactaataaattcagccaaaaaggaggacatgaaagatttaataatcttgtgcatggtattttctcaagacactaggatgttccaaggagtgagaggagaaataaattttcttaatcgagcaggccaacttttatctccaatgtaccaactccaaagatattttttgaataggagcaacgatctttttgatcaatgggggtatgaggaatcacccaaagcaagtgaactacatgggataaatgaattaatgaatgcatctgattatacgttgaaaggggttgtagtaacaaaaaatgtgattgatgactttagttctactgaaacagaaaaagtatctataacaaaaaatcttagtttaataaaaagaactggggaagtcataatgggggctaatgacgtaagtgaattagaatcacaagctcagctaatgataacatatgatacacctaagatgtgggagatgggaacaaccaaagaactggtgcaaaacacctaccaatgggtgctaaaaaatttggtaacactgaaggctcagtttcttctaggaaaagaagacatgttccaatgggatgcatttgaagcatttgaaagcataatcccccagaagatggctggccagtacagtggatttgcaagggcagtgctcaaacaaatgagagaccaagaggttatgaaaactgaccagttcataaagttgttgcctttctgtttctcaccaccaaaattaaggagcaatggggagccttatcaattcttgaggcttatattgaagggaggaggagaaaatttcatcgaagtaaggaaagggtcccctctattctcctacaatccacaaacagaagtcctaactatatgcggcagaatgatgtcattaaaagggaaaattgaagatgaagaaaggaatagatcaatggggaatgcagtattggcaggctttctcgttagtggcaagtatgacccagatcttggagatttcaaaactattgaagaacttgaaaagctaaaaccgggggagaaagcaaacatcttactttatcaaggaaagcccgttaaagtagttaaaaggaaaagatatagtgctttatccaatgacatttcacaaggaattaagagacaaagaatgacagttgagtccatggggtgggccttgagctaatataaatttatccattaattcaataaacacaattgagtgaaaaatgctcgtgtttctact

SEQ ID NO: 65 - DNA, 2308 bp, synthetic construct
  misc_feature 1..2308: note = Synthetic Sequence
  source 1..2308: mol_type = other DNA
agcagaagcggtgcgtttgatttgccataatggatacttttattacaagaaacttccagactacaataatacaaaaggccaaaaacacaatggcagaatttagtgaagatcctgaattacaaccagcaatgctattcaacatctgcgtccatctggaggtctgctatgtaataagtgatatgaattttcttgatgaagaaggaaaagcatatacagcattagaaggacaaggaaaagaacaaaacttgagaccacaatatgaagtgattgagggaatgccaagaaacatagcatggatggttcaaagatccttagcccaagagcatgggatagagactccaaggtatctggctgatttgttcgattataaaactaagaggtttatagaagttggaataacaaagggattggctgatgattacttctggaaaaagaaagaaaagctggggaatagcatggaactgatgatattcagctacaatcaagactattcgttaagtaatgaatcctcattggatgaggaaggaaaagggagagtgctaagcagactcacagaacttcaggctgagttaagtctgaaaaatctatggcaagttctcataggagaagaagatattgaaaaaggaattgacttcaaacttggacaaacaatatctaaactaagggatatatctgttccagctggtttctccaattttgaaggaatgaggagctacatagacaatatagatcctaaaggagcaatagagagaaatctagcaaggatgtctcccttagtatcagttacacctaaaaagttgaaatgggaggacctaagaccaatagggcctcacatttacaaccatgagctaccagaagttccatataatgcctttcttctaatgtctgatgaattggggctggctaatatgactgaagggaagtccaagaaaccgaagaccttagccaaagagtgcctagaaaagtactcaacactacgggatcaaactgacccaatattaataatgaaaagcgaaaaagctaatgaacacttcctatggaaactgtggagggactgtgtaaatacaataagtaatgaggaaacaagtaacgaattacagaaaaccaattatgccaagtgggccacaggagatggattaacatatcagaaaataatgaaagaagtagcaatagatgacgaaacaatgtaccaagaagagcccaaaatacctaacaaatgtagagtggctgcttgggttcaaacagagatgaatctattgagcactctgacaagtaaaagggccctggatctgccagaaatagggccagacgtagcacccgtggagcatgtagggagtgaaagaaggaaatactttgttaatgaaatcaactactgtaaggcctctaccgttatgatgaagtatgtgctttttcacacttcattattaaatgaaagcaatgccagtatgggaaaatataaagtaataccaataaccaacagagtagtaaatgaaaagggagaaagttttgacatgctttatggtctagcggttaaagggcaatctcatctgaggggagatactgatgttgtaacagttgtgactttcgaatttagtagtacagatcccagagtggactcaggaaagtggccaaaatatactgtatttagaattggctccttatttgtgagtgggagggaaaaatctgtatacctatattgccgagtgaatggtacaaataagatccaaatgaaatggggaatggaagctagaagatgtctgcttcaatcaatgcaacaaatggaagcaattgttgaacaagaatcatcgatacaaggatatgacatgaccaaagcttgtttcaagggagacagagtgaatagtccaaaaactttcagtattgggactcaagaaggaaaactagtgaaaggatcctttgggaaagcactaagagtaatattcaccaaatgtttgatgcactatgtatttggaaatgcccaattggaggggtttagtgccgaatctaggagacttctactgttaattcaggcattaaaagacagaaagggcccttgggtattcgacttagagggaatgtattctggaatagaagaatgtattagtaacaacccttgggtaatacagagtgcatactggtttaatgaatggttgggctttgaaaaagaggggagtaaagtattagaatcagtagatgaaataatggatgaatgaaagaagggcatagtgctcaatttggtactattttgttcattatgtatctaaacatccaatataaagaattgagaattaaaaatgcacgtgtttctact

SEQ ID NO: 66 - DNA, 1843 bp, synthetic construct
  misc_feature 1..1843: note = Synthetic Sequence
  source 1..1843: mol_type = other DNA
agcagaagcacagcattttcttgtgaacttcaagtaccaacaaaaaactgaaaatcaaaatgtccaacatggacattgacggcatcaacactggaataattgacaaaacaccagaagaaataacttccggaaccagtggggcaaccagaccaatcatcagaccagcaacccttgcctcaccaagcaacaaacgaaccagaaacccatccccggaaagggcaaccacaagcagtgaagctgatgtcggaaggaaaacccaaaagaaacaaactccgacagagataaagaagagcgtctacaatatggtagtgaaactgggtgaattctacaaccagatgatggtcaaagctggactcaacgatgacatggagagaaacctaatccaaaatgcacatgctgtggaaagaattctattggctgctactgatgacaagaaaactgaattccaaaagaaaaagaatgccagagacgtcaaagaagggaaagaagaaatagaccataacaaaacaggaggcaccttttacaagatggtaagagataataaaaccatctacttcagccctataagaattacctttttaaaagaagaggtgaaaacaatgtacaaaaccaccatggggagtgacggtttcagtggactaaatcacatcatgattgggcattcacagacgaacgatgtctgtttccaaagatcaaaggcactaaaaagagttggacttgacccttcattaatcagtacttttgcaggaagcacactccccagaagatcaggtacaactggtgttgcgaccaaaggaggtggaactttagtggcagaagccattcgatttataggaagagcaatggcagacagagggctattgagagacatcagagccaagacggcctatgaaaagattcttctgaatctgaaaaacaagtgctctgcgccccaacaaaaggctctggttgatcaagtgatcggaagtagaaatccagggattgcagacatagaagatctcaccctgcttgctcgaagtatggtcgttgttaggccctctgtagcaagcaaagtggtgcttcccataagcatctatgctaaaatacctcaactggggttcaacgttgaagaatactctatggttgggtatgaagccatggctctttataatatggcaacacctgtttccatattaagaatgggagacgatgcaaaagataaatcacaattattcttcatgtcttgctttggagctgcctatgaagacctaagagttctgtctgcactaacaggcacggaattcaagcctaggtcagcattaaagtgcaaaggtttccacgttccagcaaaggagcaagtggaaggaatgggggcagctctgatgtccatcaagctccagttttgggctccaatgaccagatctggggggaatgaagtaggtggagacggagggtctggtcaaataagttgcagccccgtgtttgcagtagaaagacctattgctctaagcaagcaagctgtaagaagaatgctgtcaatgaatattgagggacgtgatgcagatgtcaaaggaaatctactcaagatgatgaatgattcaatggctaagaaaaccaatggaaatgctttcattgggaagaaaatgttccaaatatcagacaaaaacaaaaccaatcccgttgagattccaattaagcagaccatccccagtttcttctttgggagggacacagcagaggattatgatgacctcgattattaaagcaacaaaatagacactatggctgtgattgtttcagtacgtttggaatgtgggtgtttactcttattgaaataaatgtaaaaaatgctgttgtttctact

SEQ ID NO: 67 - DNA, 1098 bp, synthetic construct
  misc_feature 1..1098: note = Synthetic Sequence
  source 1..1098: mol_type = other DNA
agcagaagcagaggatttgtttagtcactggcaaacgggaaaaaatggcggacaacatgaccacaacacaaattgaggtgggtccgggagcaaccaatgccactataaactttgaagcaggaattttggagtgctatgaaaggctttcatggcaaagagcccttgactaccctggtcaagaccgcctaaacagactaaagagaaaattagaatcaagaataaagactcacaacaaaagtgagcctgaaagtaaaaggatgtctcttgaagagagaaaagcaattggggtaaaaatgatgaaagtgctcctatttatgaacccatctgctggaattgaagggtttgagccatactgtatgaaaaattcctccaatagcaactgcccaaactgcaattgggccgattaccctccaacatcaggaaagtgccttgatgacatagaagaagaaccggagaatgttgatgacccaactgaaatagtattaagggacatgaacaacaaagatgcaaggcaaaagataaaagaggaagtaaacactcagaaagaagggaagttccgtttgacaatacaaagggatatacgtaatgtgttgtccttgagagtgttggtaaacggaacattcctcaagcaccctaatggatacaagtccttatcaactctgcatagattgaatgcatatgaccagagtggaaggcttgttgctaaacttgttgctactgatgatcttacagtggaggatgaagaagatggccatcggatcctcaactcactcttcgagcgttttaatgaaggacattcaaagccaattcgagcagctgaaactgcggtgggagtcttatcccaatttggtcaagagcaccgattatcaccagaggagggagacaattagactggttacggaagaactttatcttttaagtaaaagaattgatgataacatattgttccacaaaacagtaatagctaacagctccataatagctgacatgattgtatcattatcattattggaaacattgtatgagatgaaggatgtggttgaagtgtacagcaggcagtgcttgtgaatttaaaataaaaatcctcttgttactact

SEQ ID NO: 68 - protein, 752 aa, synthetic construct
  REGION 1..752: note = Synthetic Sequence
MNINPYFLFIDVPIQAAISTTFPYTGVPPYSHGTGTGYTIDTVIRTHEYSNKGKQYISDVTGCTMVDPTNGPLPEDNEPSAYAQLDCVLEALDRMDEEHPGLFQAASQNAMEALMVTTVDKLTQGRQTFDWTVCRNQPAATALNTTITSFRLNDLNGADKGGLVPFCQDIIDSLDRPEMTFFSVKNIKKKLPAKNRKGFLIKRIPMKVKDRITRVEYIKRALSLNTMTKDAERGKLKRRAIATAGIQIRGFVLVVENLAKNICENLEQSGLPVGGNEKKAKLSNAVAKMLSNCPPGGISMTVTGDNTKWNECLNPRIFLAMTERITRDSPIWFRDFCSIAPVLFSNKIARLGKGFMITSKTKRLKAQIPCPDLFSIPLERYNEETRAKLKKLKPFFNEEGTASLSPGMMMGMFNMLSTVLGVAALGIKNIGNKEYLWDGLQSSDDFALFVNAKDEETCMEGINDFYRTCKLLGINMSKKKSYCNETGMFEFTSMFYRDGFVSNFAMELPSFGVAGVNESADMAIGMTIIKNNMINNGMGPATAQTAIQLFIADYRYTYKCHRGDSKVEGKRMKIIKELWENTKGRDGLLVADGGPNIYNLRNLHIPEIVLKYNLMDPEYKGRLLHPQNPFVGHLSIEGIKEADITPAHGPVKKMDYDAVSGTHSWRTKRNRSILNTDQRNMILEEQCYAKCCNLFEACFNSASYRKPVGQHSMLEAMAHRLRMDARLDYESGRMSKDDFEKAMAHLGEIGYI

SEQ ID NO: 69 - protein, 770 aa, synthetic construct
  REGION 1..770: note = Synthetic Sequence
MTLAKIELLKQLLRDNEAKTVLKQTTVDQYNIIRKFNTSRIEKNPSLRMKWAMCSNFPLALTKGDMANRIPLEYKGIQLKTNAEDIGTKGQMCSIAAVTWWNTYGPIGDTEGFEKVYESFFLRKMRLDNATWGRITFGPVERVRKRVLLNPLTKEMPPDEASNVIMEILFPKEAGIPRESTWIHRELIKEKREKLKGTMITPIVLAYMLERELVARRRFLPVAGATSAEFIEMLHCLQGENWRQIYHPGGNKLTESRSQSMIVACRKIIRRSIVASNPLELAVEIANKTVIDTEPLKSCLAAIDGGDVACDIMRAALGLKIRQRQRFGRLELKRISGRGFKNDEEILIGNGTIQKIGIWDGEEEFHVRCGECRGILKKSKMRMEKLLINSAKKEDMKDLIILCMVFSQDTRMFQGVRGEINFLNRAGQLLSPMYQLQRYFLNRSNDLFDQWGYEESPKASELHGINELMNASDYTLKGVVVTKNVIDDFSSTETEKVSITKNLSLIKRTGEVIMGANDVSELESQAQLMITYDTPKMWEMGTTKELVQNTYQWVLKNLVTLKAQFLLGKEDMFQWDAFEAFESIIPQKMAGQYSGFARAVLKQMRDQEVMKTDQFIKLLPFCFSPPKLRSNGEPYQFLRLILKGGGENFIEVRKGSPLFSYNPQTEVLTICGRMMSLKGKIEDEERNRSMGNAVLAGFLVSGKYDPDLGDFKTIEELEKLKPGEKANILLYQGKPVKVVKRKRYSALSNDISQGIKRQRMTVESMGWALS

SEQ ID NO: 70 - protein, 726 aa, synthetic construct
  REGION 1..726: note = Synthetic Sequence
MDTFITRNFQTTIIQKAKNTMAEFSEDPELQPAMLFNICVHLEVCYVISDMNFLDEEGKAYTALEGQGKEQNLRPQYEVIEGMPRNIAWMVQRSLAQEHGIETPRYLADLFDYKTKRFIEVGITKGLADDYFWKKKEKLGNSMELMIFSYNQDYSLSNESSLDEEGKGRVLSRLTELQAELSLKNLWQVLIGEEDIEKGIDFKLGQTISKLRDISVPAGFSNFEGMRSYIDNIDPKGAIERNLARMSPLVSVTPKKLKWEDLRPIGPHIYNHELPEVPYNAFLLMSDELGLANMTEGKSKKPKTLAKECLEKYSTLRDQTDPILIMKSEKANEHFLWKLWRDCVNTISNEETSNELQKTNYAKWATGDGLTYQKIMKEVAIDDETMYQEEPKIPNKCRVAAWVQTEMNLLSTLTSKRALDLPEIGPDVAPVEHVGSERRKYFVNEINYCKASTVMMKYVLFHTSLLNESNASMGKYKVIPITNRVVNEKGESFDMLYGLAVKGQSHLRGDTDVVTVVTFEFSSTDPRVDSGKWPKYTVFRIGSLFVSGREKSVYLYCRVNGTNKIQMKWGMEARRCLLQSMQQMEAIVEQESSIQGYDMTKACFKGDRVNSPKTFSIGTQEGKLVKGSFGKALRVIFTKCLMHYVFGNAQLEGFSAESRRLLLLIQALKDRKGPWVFDLEGMYSGIEECISNNPWVIQSAYWFNEWLGFEKEGSKVLESVDEIMDE

SEQ ID NO: 71 - protein, 560 aa, synthetic construct
  REGION 1..560: note = Synthetic Sequence
MSNMDIDGINTGIIDKTPEEITSGTSGATRPIIRPATLASPSNKRTRNPSPERATTSSEADVGRKTQKKQTPTEIKKSVYNMVVKLGEFYNQMMVKAGLNDDMERNLIQNAHAVERILLAATDDKKTEFQKKKNARDVKEGKEEIDHNKTGGTFYKMVRDNKTIYFSPIRITFLKEEVKTMYKTTMGSDGFSGLNHIMIGHSQTNDVCFQRSKALKRVGLDPSLISTFAGSTLPRRSGTTGVATKGGGTLVAEAIRFIGRAMADRGLLRDIRAKTAYEKILLNLKNKCSAPQQKALVDQVIGSRNPGIADIEDLTLLARSMVVVRPSVASKVVLPISIYAKIPQLGFNVEEYSMVGYEAMALYNMATPVSILRMGDDAKDKSQLFFMSCFGAAYEDLRVLSALTGTEFKPRSALKCKGFHVPAKEQVEGMGAALMSIKLQFWAPMTRSGGNEVGGDGGSGQISCSPVFAVERPIALSKQAVRRMLSMNIEGRDADVKGNLLKMMNDSMAKKTNGNAFIGKKMFQISDKNKTNPVEIPIKQTIPSFFFGRDTAEDYDDLDY

SEQ ID NO: 72 - protein, 281 aa, synthetic construct
  REGION 1..281: note = Synthetic Sequence
MADNMTTTQIEVGPGATNATINFEAGILECYERLSWQRALDYPGQDRLNRLKRKLESRIKTHNKSEPESKRMSLEERKAIGVKMMKVLLFMNPSAGIEGFEPYCMKNSSNSNCPNCNWADYPPTSGKCLDDIEEEPENVDDPTEIVLRDMNNKDARQKIKEEVNTQKEGKFRLTIQRDIRNVLSLRVLVNGTFLKHPNGYKSLSTLHRLNAYDQSGRLVAKLVATDDLTVEDEEDGHRILNSLFERFNEGHSKPIRAAETAVGVLSQFGQEHRLSPEEGDN

SEQ ID NO: 73 - DNA, 1111 bp, synthetic construct
  misc_feature 1..1111: note = Synthetic Sequence
  source 1..1111: mol_type = other DNA
agcagaagcacgcactttcttaaaatgtcgctgtttggagacacaattgcctacctgctttcattgacagaagatggagaaggcaaagcagaactagcagaaaaattacactgttggttcggtgggaaagaatttgacctagactctgccttggaatggataaaaaacaaaagatgcttaactgacatacagaaagcactaattggcgcctctatctgctttttaaaacccaaagaccaggaaagaaaaagaagattcatcacagagcccctatcaggaatggggacaacagcaacaaaaaagaagggcctgattctagctgagagaaaaatgagaagatgtgtgagcttccatgaagcatttgaaatagcagaaggccatgaaagctcagcgttactatattgtctcatggtcatgtacctgaatcctggaaattattcaatgcaagtaaaactaggaacgctctgtgctttgtgcgaaaaacaagcatcacattcacacagggctcatagcagagcagcgagatcttcagtgcctggagtgagacgggaaatgcagatggtctcagctatgaacacagcaaaaacaatgaatggaatgggaaaaggagaagacgttcaaaaactggcagaagaactgcaaagcaacattggagtattgagatctcttggggcaagtcaaaagaatggggaaggaattgcaaaggatgtaatggaagtgctaaagcagagctctatgggaaattcagctcttgtgaagaaatacctataatgctcgaaccatttcagattctttcaatttgttagatagctaaaaggggccaaataaagagacaataaacagagaggtatcaattttgagacacagttaccaaaaagaaatccaggccaaagaagcaatgaaggaagtactctctgacaacatggaggtattgagtgaccacatagtaattgaggggctttctgctgaagagataataaaaatgggtgaaacagttttggaggtagaagaattgcattaaattcaatttttactgtacttcttactatgcatttaagcaaattgtaatcaatgtcagcaaataaactggaaaaagtgcgttgtttctact

SEQ ID NO: 74 - protein, 109 aa, synthetic construct; identical to SEQ ID NO: 2
  REGION 1..109: note = Synthetic Sequence

SEQ ID NO: 75 - skipped

SEQ ID NO: 76 - protein, 15 aa, synthetic construct
  REGION 1..15: note = Synthetic Sequence
GGGGSGGGGSGGGGS

SEQ ID NO: 77 - protein, 1273 aa, synthetic construct
  REGION 1..1273: note = Synthetic Sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 78 - protein, 11 aa, synthetic construct
  REGION 1..11: note = Synthetic Sequence
MLEPFQILSIC

SEQ ID NO: 79 - DNA, 1423 bp, synthetic construct
  misc_feature 1..1423: note = Synthetic Sequence
  source 1..1423: mol_type = other DNA
agcaaaagcaggtagatattgaaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattggaactcatcctagctccagtgctggtctgaaaaatgatcttcttgaaaatttgcaggcctatcagaaacgaatgggggtgcagatgcaacggttcaagtgatcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctatagagctggagtaaaaaactaccttgtttctact

SEQ ID NO: 80 - DNA, 1774 bp, synthetic construct
  misc_feature 1..1774: note = Synthetic Sequence
  source 1..1774: mol_type = other DNA
agcaaaagcagggtgacaaaaacataatggatccaaacactgtgtcaagctttcaagtagattgctttctttggcatgtccgcaaacgagttgcagaccaagaactaggcgatcccccattccttgatcggcttcgccgagatcagaaatccctaagaggaaggggcagtactctcggtctggacatcaagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcaaaatggaccaggcgatcatggataagaacatcatactgaaagcgaacttcagtgtgatttttgaccggctggagactctaatattgctaagggctttcaccgaagagggagcaattgttggcgaaatttcaccattgccttctctcccgggacatactgctgaggttgtcaaaaatgcagttggagtcctaatcggaggacttgaatggaacgataacacagttcgagtctctgaaactctacagagattcgcttggagaagcagtaatgagaacgggagacctccactgactccaaaacagaaacgagaaatggcgggaacaattaggtcagaagttggtagtggaaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctggatcaggtgcaactaactttagtcttcttaaacaagctggtgatgttgaagaaaatcctggacctaatacagtgtctagcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttagtactaaaaaacacccttgtttctact

SEQ ID NO: 81 - DNA, 1482 bp, synthetic construct
  misc_feature 1..1482: note = Synthetic Sequence
  source 1..1482: mol_type = other DNA
agcaaaagcaggtagatattgaaagatgagtcttctaaccgaggtcgaaacttacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattggaactcatcctagctccagtgctggtctgaaaaatgatcttcttgaaaatttgcaagcctatcagaaacgaatgggggtgcagatgcaacggttcaagggatcaggtgcaactaactttagtcttcttaaacaagctggtgatgttgaagaaaatcctggacctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctatagagctggagtaaaaaactaccttgtttctact

SEQ ID NO: 82 - DNA, 1424 bp, synthetic construct
  misc_feature 1..1424: note = Synthetic Sequence
  source 1..1424: mol_type = other DNA
agcaaaagcaggtagatattgaaagatgagtcttctaaccgaggtcgaaacttacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagttaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattggaactcatcctagctccagtgctggtctgaaaaatgatcttcttgaaaatttgcaagcctatcagaaacgaatgggggtgcagatgcaacggttcaagtaatgcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctatagagctggagtaaaaaactaccttgtttctact

SEQ ID NO: 83 - DNA, 1472 bp, synthetic construct
  misc_feature 1..1472: note = Synthetic Sequence
  source 1..1472: mol_type = other DNA
agcagaagcacgcactttcttaaaatgtcgctgtttggagacacaattgcctacctgctttcattgacagaagatggagaaggcaaagcagaactagcagaaaaattacactgttggttcggtgggaaagaatttgacctagactctgccttggaatggataaaaaacaaaagatgcttaactgacatacagaaagcactaattggcgcctctatctgctttttaaaacccaaagaccaggaaagaaaaagaagattcatcacagagcccctatcaggaatggggacaacagcaacaaaaaagaagggcctgattctagctgagagaaaaatgagaagatgtgtgagcttccatgaagcatttgaaatagcagaaggccatgaaagctcagcgttactatattgtctcatggtcatgtacctgaatcctggaaattattcaatgcaagtaaaactaggaacgctctgtgctttgtgcgaaaaacaagcatcacattcacacagggctcatagcagagcagcgagatcttcagtgcctggagtgagacgggaaatgcagatggtctcagctatgaacacagcaaaaacaatgaatggaatgggaaaaggagaagacgttcaaaaactggcagaagaactgcaaagcaacattggagtattgagatctcttggggcaagtcaaaagaatggggaaggaattgcaaaggatgtaatggaagtgctaaagcagagctctatgggaaattcagctcttgtgaagaaatacctataatgctcgaatcaattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaacagtttaaattcaatttttactgtacttcttactatgcatttaagcaaattgtaatcaatgtcagcaaataaactggaaaaagtgcgttgtttctact

SEQ ID NO: 84 - DNA, 1508 bp, synthetic construct
  misc_feature 1..1508: note = Synthetic Sequence
  source 1..1508: mol_type = other DNA
agcagaagcacgcactttcttaaaatgtcgctgtttggagacacaattgcctacctgctttcattgacagaagatggagaaggcaaagcagaactagcagaaaaattacactgttggttcggtgggaaagaatttgacctagactctgccttggaatggataaaaaacaaaagatgcttaactgacatacagaaagcactaattggcgcctctatctgctttttaaaacccaaagaccaggaaagaaaaagaagattcatcacagagcccctatcaggaatggggacaacagcaacaaaaaagaagggcctgattctagctgagagaaaaatgagaagatgtgtgagcttccatgaagcatttgaaatagcagaaggccatgaaagctcagcgttactatattgtctcatggtcatgtacctgaatcctggaaattattcaatgcaagtaaaactaggaacgctctgtgctttgtgcgaaaaacaagcatcacattcacacagggctcatagcagagcagcgagatcttcagtgcctggagtgagacgggaaatgcagatggtctcagctatgaacacagcaaaaacaatgaatggaatgggaaaaggagaagacgttcaaaaactggcagaagaactgcaaagcaacattggagtattgagatctcttggggcaagtcaaaagaatggggaaggaattgcaaaggatgtaatggaagtgctaaagcagagctctatgggaaattcagctcttgtgaagaaatacctataatgctcgaaccatttcagattctttcaattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaacagttttggaggtagaagaattgcattaaattcaatttttactgtacttcttactatgcatttaagcaaattgtaatcaatgtcagcaaataaactggaaaaagtgcgttgtttctact

SEQ ID NO: 85 - DNA, 1165 bp, synthetic construct
  misc_feature 1..1165: note = Synthetic Sequence
  source 1..1165: mol_type = other DNA
agcaaaagcagggtgacaaaaacataatggatccaaacactgtgtcaagctttcaagtagattgctttctttggcatgtccgcaaacgagttgcagaccaagaactaggcgatcccccattccttgatcggcttcgccgagatcagaaatccctaagaggaaggggcagtactctcggtctggacatcaagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcaaaatggaccaggcgatcatggataagaacatcatactgaaagcgaacttcagtgtgatttttgaccggctggagactctaatattgctaagggctttcaccgaagagggagcaattgttggcgaaatttcaccattgccttctctcccgggacatactgctgaggttgtcaagaatgctgttggagtgctaatcggaggacttgaatggaacgacaacactgtgcgagtcagcgaaactttgcagagatttgcttggagaagctctaatgagaacgggagacctccactgactccaaagcagaaacgagacatggctggaacaattagaagcgaagttggatcaggtgcaactaactttagtcttcttaaacaagctggtgatgttgaagaaaatcctggacctaatacagtgtctagcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttagtactaaaaaacacccttgtttctact

SEQ ID NO: 86 - DNA, 1828 bp, synthetic construct
  misc_feature 1..1828: note = Synthetic Sequence
  source 1..1828: mol_type = other DNA
agcaaaagcagggtgacaaaaacataatggatccaaacactgtgtcaagctttcaagtagattgctttctttggcatgtccgcaaacgagttgcagaccaagaactaggcgatcccccattccttgatcggcttcgccgagatcagaaatccctaagaggaaggggcagtactctcggtctggacatcaagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcaaaatggaccaggcgatcatggataagaacatcatactgaaagcgaacttcagtgtgatttttgaccggctggagactctaatattgctaagggctttcaccgaagagggagcaattgttggcgaaatttcaccattgccttctctcccgggacatactgctgaggttgtcaaaaatgcagttggagtcctaatcggaggacttgaatggaacgataacacagttcgagtctctgaaactctacagagattcgcttggagaagcagtaatgagaacgggagacctccactgactccaaaacagaaacgagaaatggcgggaacaattaggtcagaagttggtagtggagaaggtagaggaagtcttctaacatgtggtgatgtagaagagaatccaggtcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctggatcaggtgcaactaactttagtcttcttaaacaagctggtgatgttgaagaaaatcctggacctaatacagtgtctagcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttagtactaaaaaacacccttgtttctact

SEQ ID NO: 87 - DNA, 1984 bp, synthetic construct
  misc_feature 1..1984: note = Synthetic Sequence
  source 1..1984: mol_type = other DNA
agcaaaagcagggtgacaaaaacataatggatccaaacactgtgtcaagctttcaagtagattgctttctttggcatgtccgcaaacgagttgcagaccaagaactaggcgatcccccattccttgatcggcttcgccgagatcagaaatccctaagaggaaggggcagtactctcggtctggacatcaagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcaaaatggaccaggcgatcatggataagaacatcatactgaaagcgaacttcagtgtgatttttgaccggctggagactctaatattgctaagggctttcaccgaagagggagcaattgttggcgaaatttcaccattgccttctctcccgggacatactgctgaggttgtcaaaaatgcagttggagtcctaatcggaggacttgaatggaacgataacacagttcgagtctctgaaactctacagagattcgcttggagaagcagtaatgagaacgggagacctccactgactccaaaacagaaacgagaaatggcgggaacaattaggtcagaagttggtagtggacctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgacccaggatcaggtgcaactaactttagtcttcttaaacaagctggtgatgttgaagaaaatcctggacctaatacagtgtctagcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttagtactaaaaaacacccttgtttctact

SEQ ID NO: 88 - DNA, 1444 bp, synthetic construct
  misc_feature 1..1444: note = Synthetic Sequence
  source 1..1444: mol_type = other DNA
agcaaaagcagggtgacaaaaacataatggatccaaacactgtgtcaagctttcaagtagattgctttctttggcatgtccgcaaacgagttgcagaccaagaactaggcgatcccccattccttgatcggcttcgccgagatcagaaatccctaagaggaaggggcagtactctcggtctggacatcaagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcaaaatggaccaggcgatcatggataagaacatcatactgaaagcgaacttcagtgtgatttttgaccggctggagactctaatattgctaagggctttcaccgaagagggagcaattgttggcgaaatttcaccattgccttctctcccgggacatactgctgaggttgtcaaaaatgcagttggagtcctaatcggaggacttgaatggaacgataacacagttcgagtctctgaaactctacagagattcgcttggagaagcagtaatgagaacgggagacctccactgactccaaaacagaaacgagaaatggcgggaacaattaggtcagaagttggtagtggagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagggatcaggtgcaactaactttagtcttcttaaacaagctggtgatgttgaagaaaatcctggacctaatacagtgtctagcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttagtactaaaaaacacccttgtttctact

SEQ ID NO: 89 - DNA, 1444 bp, synthetic construct
  misc_feature 1..1444: note = Synthetic Sequence
  source 1..1444: mol_type = other DNA
agcaaaagcagggtgacaaaaacataatggatccaaacactgtgtcaagctttcaagtagattgctttctttggcatgtccgcaaacgagttgcagaccaagaactaggcgatcccccattccttgatcggcttcgccgagatcagaaatccctaagaggaaggggcagtactctcggtctggacatcaagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcaaaatggaccaggcgatcatggataagaacatcatactgaaagcgaacttcagtgtgatttttgaccggctggagactctaatattgctaagggctttcaccgaagagggagcaattgttggcgaaatttcaccattgccttctctcccgggacatactgctgaggttgtcaaaaatgcagttggagtcctaatcggaggacttgaatggaacgataacacagttcgagtctctgaaactctacagagattcgcttggagaagcagtaatgagaacgggagacctccactgactccaaaacagaaacgagaaatggcgggaacaattaggtcagaagttggtagtggagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacccacctgaggctgaagtgcaaattgataggttgatcacaggcagacttcaaagtttgcagacatatgtgactcaacaattaattagagctgcagaaatcagagcttctgctaatcttgctgctactaaaatgtcagagggatcaggtgcaactaactttagtcttcttaaacaagctggtgatgttgaagaaaatcctggacctaatacagtgtctagcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttagtactaaaaaacacccttgtttctact

SEQ ID NO: 90 - DNA, 1375 bp, synthetic construct
  misc_feature 1..1375: note = Synthetic Sequence
  source 1..1375: mol_type = other DNA
agcaaaagcagggtgacaaaaacataatggatccaaacactgtgtcaagctttcaagtagattgctttctttggcatgtccgcaaacgagttgcagaccaagaactaggcgatcccccattccttgatcggcttcgccgagatcagaaatccctaagaggaaggggcagtactctcggtctggacatcaagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcaaaatggaccaggcgatcatggataagaacatcatactgaaagcgaacttcagtgtgatttttgaccggctggagactctaatattgctaagggctttcaccgaagagggagcaattgttggcgaaatttcaccattgccttctctcccgggacatactgctgaggttgtcaaaaatgcagttggagtcctaatcggaggacttgaatggaacgataacacagttcgagtctctgaaactctacagagattcgcttggagaagcagtaatgagaacgggagacctccactgactccaaaacagaaacgagaaatggcgggaacaattaggtcagaagttggtagtggaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatggatcaggtgcaactaactttagtcttcttaaacaagctggtgatgttgaagaaaatcctggacctaatacagtgtctagcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttagtactaaaaaacacccttgtttctact

SEQ ID NO: 91 - DNA, 1396 bp, synthetic construct
  misc_feature 1..1396: note = Synthetic Sequence
  source 1..1396: mol_type = other DNA
agcaaaagcagggtgacaaaaacataatggatccaaacactgtgtcaagctttcaagtagattgctttctttggcatgtccgcaaacgagttgcagaccaagaactaggcgatcccccattccttgatcggcttcgccgagatcagaaatccctaagaggaaggggcagtactctcggtctggacatcaagacagccacacgtgctggaaagcagatagtggagcggattctgaaagaagaatccgatgaggcacttaaaatgaccatggcctctgtacctgcgtcgcgttacctaactgacatgactcttgaggaaatgtcaagggactggtccatgctcatacccaagcagaaagtggcaggccctctttgtatcaaaatggaccaggcgatcatggataagaacatcatactgaaagcgaacttcagtgtgatttttgaccggctggagactctaatattgctaagggctttcaccgaagagggagcaattgttggcgaaatttcaccattgccttctctcccgggacatactgctgaggttgtcaaaaatgcagttggagtcctaatcggaggacttgaatggaacgataacacagttcgagtctctgaaactctacagagattcgcttggagaagcagtaatgagaacgggagacctccactgactccaaaacagaaacgagaaatggcgggaacaattaggtcagaagttggtagtggacctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccaggatcaggtgcaactaactttagtcttcttaaacaagctggtgatgttgaagaaaatcctggacctaatacagtgtctagcttccaggacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtttgaagaaataagatggttgattgaagaagtgagacacaaactgaagataacagagaatagttttgagcaaataacatttatgcaagccttacatctattgcttgaagtggagcaagagataagaactttctcgtttcagcttatttagtactaaaaaacacccttgtttctact

SEQ ID NO: 92 - protein, 229 aa, synthetic construct
  REGION 1..229: note = Synthetic Sequence
MSLLTEVETPIRNEWGCRCNGSSDPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSIELE

SEQ ID NO: 93 - protein, 478 aa, synthetic construct
  REGION 1..478: note = Synthetic Sequence
MSLLTEVETYVLSIIPSGPLKAEIAQRLEDVFAGKNTDLEVLMEWLKTRPILSPLTKGILGFVFTLTVPSERGLQRRRFVQNALNGNGDPNNMDKAVKLYRKLKREITFHGAKEISLSYSAGALASCMGLIYNRMGAVTTEVAFGLVCATCEQIADSQHRSHRQMVTTTNPLIRHENRMVLASTTAKAMEQMAGSSEQAAEAMEVASQARQMVQAMRTIGTHPSSSAGLKNDLLENLQAYQKRMGVQMQRFKGSGATNFSLLKQAGDVEENPGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSIELE

SEQ ID NO: 94 - protein, 206 aa, synthetic construct
  REGION 1..206: note = Synthetic Sequence
MPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSIELE

SEQ ID NO: 95 - protein, 203 aa, synthetic construct
  REGION 1..203: note = Synthetic Sequence
MLESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 96 - protein, 215 aa, synthetic construct
  REGION 1..215: note = Synthetic Sequence
MLEPFQILSIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVLEVEELH

SEQ ID NO: 97 - protein, 573 aa, synthetic construct
  REGION 1..573: note = Synthetic Sequence
MDPNTVSSFQVDCFLWHVRKRVADQELGDPPFLDRLRRDQKSLRGRGSTLGLDIKTATRAGKQIVERILKEESDEALKMTMASVPASRYLTDMTLEEMSRDWSMLIPKQKVAGPLCIKMDQAIMDKNIILKANFSVIFDRLETLILLRAFTEEGAIVGEISPLPSLPGHTAEVVKNAVGVLIGGLEWNDNTVRVSETLQRFAWRSSNENGRPPLTPKQKREMAGTIRSEVGSGNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSGSGATNFSLLKQAGDVEENPGPNTVSSFQDILLRMSKMQLESSSEDLNGMITQFESLKLYRDSLGEAVMRMGDLHSLQNRNEKWREQLGQKFEEIRWLIEEVRHKLKITENSFEQITFMQALHLLLEVEQEIRTFSFQLI

SEQ ID NO: 98 - protein, 370 aa, synthetic construct
  REGION 1..370: note = Synthetic Sequence
MDPNTVSSFQVDCFLWHVRKRVADQELGDPPFLDRLRRDQKSLRGRGSTLGLDIKTATRAGKQIVERILKEESDEALKMTMASVPASRYLTDMTLEEMSRDWSMLIPKQKVAGPLCIKMDQAIMDKNIILKANFSVIFDRLETLILLRAFTEEGAIVGEISPLPSLPGHTAEVVKNAVGVLIGGLEWNDNTVRVSETLQRFAWRSSNENGRPPLTPKQKRDMAGTIRSEVGSGATNFSLLKQAGDVEENPGPNTVSSFQDILLRMSKMQLESSSEDLNGMITQFESLKLYRDSLGEAVMRMGDLHSLQNRNEKWREQLGQKFEEIRWLIEEVRHKLKITENSFEQITFMQALHLLLEVEQEIRTFSFQLI

SEQ ID NO: 99 - protein, 591 aa, synthetic construct
  REGION 1..591: note = Synthetic Sequence
MDPNTVSSFQVDCFLWHVRKRVADQELGDPPFLDRLRRDQKSLRGRGSTLGLDIKTATRAGKQIVERILKEESDEALKMTMASVPASRYLTDMTLEEMSRDWSMLIPKQKVAGPLCIKMDQAIMDKNIILKANFSVIFDRLETLILLRAFTEEGAIVGEISPLPSLPGHTAEVVKNAVGVLIGGLEWNDNTVRVSETLQRFAWRSSNENGRPPLTPKQKREMAGTIRSEVGSGEGRGSLLTCGDVEENPGPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSGSGATNFSLLKQAGDVEENPGPNTVSSFQDILLRMSKMQLESSSEDLNGMITQFESLKLYRDSLGEAVMRMGDLHSLQNRNEKWREQLGQKFEEIRWLIEEVRHKLKITENSFEQITFMQALHLLLEVEQEIRTFSFQLI

SEQ ID NO: 100 - protein, 643 aa, synthetic construct
  REGION 1..643: note = Synthetic Sequence
MDPNTVSSFQVDCFLWHVRKRVADQELGDPPFLDRLRRDQKSLRGRGSTLGLDIKTATRAGKQIVERILKEESDEALKMTMASVPASRYLTDMTLEEMSRDWSMLIPKQKVAGPLCIKMDQAIMDKNIILKANFSVIFDRLETLILLRAFTEEGAIVGEISPLPSLPGHTAEVVKNAVGVLIGGLEWNDNTVRVSETLQRFAWRSSNENGRPPLTPKQKREMAGTIRSEVGSGPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPGSGATNFSLLKQAGDVEENPGPNTVSSFQDILLRMSKMQLESSSEDLNGMITQFESLKLYRDSLGEAVMRMGDLHSLQNRNEKWREQLGQKFEEIRWLIEEVRHKLKITENSFEQITFMQALHLLLEVEQEIRTFSFQLI

SEQ ID NO: 101 - protein, 463 aa, synthetic construct
  REGION 1..463: note = Synthetic Sequence
MDPNTVSSFQVDCFLWHVRKRVADQELGDPPFLDRLRRDQKSLRGRGSTLGLDIKTATRAGKQIVERILKEESDEALKMTMASVPASRYLTDMTLEEMSRDWSMLIPKQKVAGPLCIKMDQAIMDKNIILKANFSVIFDRLETLILLRAFTEEGAIVGEISPLPSLPGHTAEVVKNAVGVLIGGLEWNDNTVRVSETLQRFAWRSSNENGRPPLTPKQKREMAGTIRSEVGSGASALGKLQDVVNQNAQALNTLVKQLSSNFGAISSVLNDILSRLDKVEAEVQIDRLITGRLQSLQTYVTQQLIRAAEIRASANLAATKMSEGSGATNFSLLKQAGDVEENPGPNTVSSFQDILLRMSKMQLESSSEDLNGMITQFESLKLYRDSLGEAVMRMGDLHSLQNRNEKWREQLGQKFEEIRWLIEEVRHKLKITENSFEQITFMQALHLLLEVEQEIRTFSFQLI

SEQ ID NO: 102 - protein, 463 aa, synthetic construct
  REGION 1..463: note = Synthetic Sequence
MDPNTVSSFQVDCFLWHVRKRVADQELGDPPFLDRLRRDQKSLRGRGSTLGLDIKTATRAGKQIVERILKEESDEALKMTMASVPASRYLTDMTLEEMSRDWSMLIPKQKVAGPLCIKMDQAIMDKNIILKANFSVIFDRLETLILLRAFTEEGAIVGEISPLPSLPGHTAEVVKNAVGVLIGGLEWNDNTVRVSETLQRFAWRSSNENGRPPLTPKQKREMAGTIRSEVGSGASALGKLQDVVNQNAQALNTLVKQLSSNFGAISSVLNDILSRLDPPEAEVQIDRLITGRLQSLQTYVTQQLIRAAEIRASANLAATKMSEGSGATNFSLLKQAGDVEENPGPNTVSSFQDILLRMSKMQLESSSEDLNGMITQFESLKLYRDSLGEAVMRMGDLHSLQNRNEKWREQLGQKFEEIRWLIEEVRHKLKITENSFEQITFMQALHLLLEVEQEIRTFSFQLI

SEQ ID NO: 103 - protein, 440 aa, synthetic construct
  REGION 1..440: note = Synthetic Sequence
MDPNTVSSFQVDCFLWHVRKRVADQELGDPPFLDRLRRDQKSLRGRGSTLGLDIKTATRAGKQIVERILKEESDEALKMTMASVPASRYLTDMTLEEMSRDWSMLIPKQKVAGPLCIKMDQAIMDKNIILKANFSVIFDRLETLILLRAFTEEGAIVGEISPLPSLPGHTAEVVKNAVGVLIGGLEWNDNTVRVSETLQRFAWRSSNENGRPPLTPKQKREMAGTIRSEVGSGKNFTTAPAICHDGKAHFPREGVFVSNGTHWFVTQRNFYEPQIITTDNTFVSGNCDVVIGIVNNTVYDGSGATNFSLLKQAGDVEENPGPNTVSSFQDILLRMSKMQLESSSEDLNGMITQFESLKLYRDSLGEAVMRMGDLHSLQNRNEKWREQLGQKFEEIRWLIEEVRHKLKITENSFEQITFMQALHLLLEVEQEIRTFSFQLI

SEQ ID NO: 104 - protein, 447 aa, synthetic construct
  REGION 1..447: note = Synthetic Sequence
MDPNTVSSFQVDCFLWHVRKRVADQELGDPPFLDRLRRDQKSLRGRGSTLGLDIKTATRAGKQIVERILKEESDEALKMTMASVPASRYLTDMTLEEMSRDWSMLIPKQKVAGPLCIKMDQAIMDKNIILKANFSVIFDRLETLILLRAFTEEGAIVGEISPLPSLPGHTAEVVKNAVGVLIGGLEWNDNTVRVSETLQRFAWRSSNENGRPPLTPKQKREMAGTIRSEVGSGPLQPELDSFKEELDKYFKNHTSPDVDLGDISGINASVVNIQKEIDRLNEVAKNLNESLIDLQELGKYEQYIKWPGSGATNFSLLKQAGDVEENPGPNTVSSFQDILLRMSKMQLESSSEDLNGMITQFESLKLYRDSLGEAVMRMGDLHSLQNRNEKWREQLGQKFEEIRWLIEEVRHKLKITENSFEQITFMQALHLLLEVEQEIRTFSFQLI

SEQ ID NO: 105 - DNA, 1096 bp, synthetic construct
  misc_feature 1..1096: note = Synthetic Sequence
  source 1..1096: mol_type = other DNA
agcaaaagcaggtagatattgaaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagctaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctaatcttgattctaaggttggtggtaattataattacctgtatagattgtttaggaagtctaatctcaaaccttttgagagagcaggtgcaggtgattacaaggatgacgacgataagtaataggatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctact

SEQ ID NO: 106 - DNA, 1108 bp, synthetic construct
  misc_feature 1..1108: note = Synthetic Sequence
  source 1..1108: mol_type = other DNA
agcaaaagcaggtagatattgaaagatgagtcttctaaccgaggtcgaaacgtacgtactctctatcatcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacaaagcagctaaactgtataggaagctcaagagggagataacattccatggggccaaagaaatctcactcagttattctgctggtgcacttgccagttgtatgggcctcatatacaacaggatgggggctgtgaccactgaagtggcatttggcctggtatgtgcaacctgtgaacagattgctgactcccagcatcggtctcataggcaaatggtgacaacaaccaatccactaatcagacatgagaacagaatggttttagccagcactacagctaaggctatggagcaaatggctggatcgagtgagcaagcagcagaggccatggaggttgctagtcaggctagacaaatggtgcaagcgatgagaaccattgggactcatcctagctccagtgctggtctgaaaaatgatcttcttgaaaatttgcaggcctatcagaaacgaatgggggtgcagatgcaacggttcaagtgatcctatcgatctccaagaacttggaaagtatgagcagtatataaaatggccatggtacatttggctaggttttatagctggcttgattgccatagtagcaggtgcaggtgattacaaggatgacgacgataagtaataggatcgtctttttttcaaatgcatttaccgtcgctttaaatacggactgaaaggagggccttctacggaaggagtgccaaagtctatgagggaagaatatcgaaaggaacagcagagtgctgtggatgctgacgatggtcattttgtcagcatagagctggagtaaaaaactaccttgtttctact

SEQ ID NO: 107 - DNA, 976 bp, synthetic construct; identical to SEQ ID NO: 53
  misc_feature 1..976: note = Synthetic Sequence
  source 1..976: mol_type = other DNA

SEQ ID NO: 108 - protein, 119 aa, synthetic construct
  REGION 1..119: note = Synthetic Sequence
PNTVSSFQDILLRMSKMQLESSSEDLNGMITQFESLKLYRDSLGEAVMRMGDLHSLQNRNEKWREQLGQKFEEIRWLIEEVRHKLKITENSFEQITFMQALHLLLEVEQEIRTFSFQLI

SEQ ID NO: 109 - DNA, 30 bp, synthetic construct
  misc_feature 1..30: note = Synthetic Sequence
  source 1..30: mol_type = other DNA
atggatccaaacactgtgtcaagctttcaa

SEQ ID NO: 110 - DNA, 24 bp, synthetic construct
  misc_feature 1..24: note = Synthetic Sequence
  source 1..24: mol_type = other DNA
cctaatacagtgtctagcttccag

SEQ ID NO: 111 - DNA, 188 bp, synthetic construct
  misc_feature 1..188: note = Synthetic Sequence
  source 1..188: mol_type = other DNA
gacatactgctgaggttgtcaagaatgctgttggagtgctaatcggaggacttgaatggaacgacaacactgtgcgagtcagcgaaactttgcagagatttgcttggagaagctctaatgagaacgggagacctccactgactccaaagcagaaacgagacatggctggaacaattagaagcgaagtt

SEQ ID NO: 112 - DNA, 188 bp, synthetic construct
  misc_feature 1..188: note = Synthetic Sequence
  source 1..188: mol_type = other DNA
gacatactgctgaggatgtcaaaaatgcagttggagtcctcatcggaggacttgaatggaatgataacacagttcgagtctctgaaactctacagagattcgcttggagaagcagtaatgagaatgggagacctccactcactccaaaacagaaacgagaaatggcgggaacaattaggtcagaagtt

SEQ ID NO: 113 - protein, 282 aa, synthetic construct
  REGION 1..282: note = Synthetic Sequence
GSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGSGATNFSLLKQAGDVEENPGP

SEQ ID NO: 114 - DNA, 846 bp, synthetic construct
  misc_feature 1..846: note = Synthetic Sequence
  source 1..846: mol_type = other DNA
ggtagtggagaaggtagaggaagtcttctaacatgtggtgatgtagaagagaatccaggtcctgtatctaaaggtgaagaattattcactggtgttgtcccaattttggttgaattagatggtgatgttaatggtcataaattttctgtctccggtgaaggtgaaggtgatgctacttacggtaaattgaccttaaaatttatttgtactactggtaaattgccagttccatggccaaccttagtcactactttaacttatggtgttcaatgtttttcaagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaacttgaatacaactataactctcacaatgtttacatcatggctgacaaacaaaagaatggtatcaaagttaacttcaaaattagacacaacattgaagatggtagcgttcaattagctgaccattatcaacaaaatactccaattggtgatggtccagtcttgttaccagacaaccattacttatccacacaatctgccttatccaaagatccaaacgaaaagagagatcacatggtcttgttagaatttgttactgctgctggtattacccttggtatggatgaattgtacaaatcaggatcaggtgcaactaactttagtcttcttaaacaagctggtgatgttgaagaaaatcctggacct

SEQ ID NO: 115 - protein, 275 aa, synthetic construct
  REGION 1..275: note = Synthetic Sequence
FVFLVLLPLVSSQPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSGYIPEAPRDGQAYVRKDGEWVLLSTFLGSTTNIMITTIIIVIIVILLSLIAVGLLLYCKAR

SEQ ID NO: 116 - protein, 773 aa, synthetic construct
  REGION 1..773: note = Synthetic Sequence
MKTIIALSYILSLVFAQKPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGGGGSQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGTSSACIRGSSSSFFSRLNWLTHLNYKYPALNVTMPNKEQFDKLYIWGVHHPGTDKDQIFPYAQSSGRITVSTKRSQQAVIPNIGSRPRIRGIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIESIRNETYDHNVYRDEALNNRFQIKGVELKSGYKDWILWISFAISCFLLCVALLGFIMWACQKGNIRCNICI

SEQ ID NO: 117 - protein, 625 aa, synthetic construct
  REGION 1..625: note = Synthetic Sequence
MKTIIALSYILCLVFAQKIPVSTYMLTNSELLSLINDMPITNDQKKLMSNNVQIVRQQSYSIMSIIKEAAAPGAAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGTSSACIRGSSSSFFSRLNWLTHLNYKYPALNVTMPNKEQFDKLYIWGVHHPGTDKDQIFPYAQSSGRITVSTKRSQQAVIPNIGSRPRIRGIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIESIRNETYDHNVYRDEALNNRFQIKGVELKSGYKDWILWISFAISCFLLCVALLGFIMWACQKGNIRCNICI

SEQ ID NO: 118 - protein, 673 aa, synthetic construct
  REGION 1..673: note = Synthetic Sequence
MKTIIALSYILCLVFAQKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPAAAPGAAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGTSSACIRGSSSSFFSRLNWLTHLNYKYPALNVTMPNKEQFDKLYIWGVHHPGTDKDQIFPYAQSSGRITVSTKRSQQAVIPNIGSRPRIRGIPSRISIYWTIVKPGDILLINSTGNLIAPRGYFKIRSGKSSIMRSDAPIGKCKSECITPNGSIPNDKPFQNVNRITYGACPRYVKHSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIESIRNETYDHNVYRDEALNNRFQIKGVELKSGYKDWILWISFAISCFLLCVALLGFIMWACQKGNIRCNICI

SEQ ID NO: 119 - protein, 234 aa, synthetic construct
  REGION 1..234: note = Synthetic Sequence
GSGEGRGSLLTCGDVEENPGPFVFLVLLPLVSSQCVNLTTRTQLPPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCGSGATNFSLLKQAGDVEENPGP